(19) United States
(12) Patent Application Publication
(10) Pub. No.: US 2013/0005597 A1
(43) Pub. Date: Jan. 3, 2013

(54) METHODS AND COMPOSITIONS FOR ANALYSIS OF CLEAR CELL RENAL CELL CARCINOMA (CCRCC)

(76) Inventors: W. Kimryn Rathmell, Chapel Hill, NC (US); A. Rose Brannon, Chapel Hill, NC (US); Gyan Bhanot, Piscataway, NJ (US); Anupama Reddy, Boston, MA (US)

(21) Appl. No.: 13/516,105

(22) PCT Filed: Dec. 20, 2010
(86) PCT No.: PCT/US10/61301

§ 371 Date: Sep. 19, 2012

Related U.S. Application Data
(60) Provisional application No. 61/287,986, filed on Dec. 18, 2009.

(51) Int. Cl. C40B 30/04; C40B 40/10; C40B 40/06

(52) U.S. Cl. 506/9; 506/16; 506/18

(57) ABSTRACT

Methods for generating a prognostic signature for a subject with clear cell renal cell carcinoma (ccRCC) are disclosed. The methods include determining expression levels for three or more genes disclosed in ccRCC cells obtained from the subject, wherein the determining provides a prognostic signature for the subject. Also provided are methods for assessing risk of an adverse outcome of a subject clear cell renal cell carcinoma (ccRCC), method for predicting a clinical outcome of a treatment in a subject diagnosed with clear cell renal cell carcinoma (ccRCC), and arrays that include polynucleotides that hybridize to at least three genes disclosed or that include specific peptide or polypeptide gene products of at least three of the genes disclosed.

### CROSS REFERENCE TO RELATED APPLICATION

The presently disclosed subject matter claims the benefit of U.S. Provisional Patent Application Ser. No. 61/287,986, filed Dec. 18, 2009; the disclosure of which is incorporated herein by reference in its entirety.

### GOVERNMENT INTEREST

This invention was made with government support under Grant No. PHY05-51164 from the National Science Foundation. The government has certain rights in the invention.

### TECHNICAL FIELD

The presently disclosed subject matter relates in some embodiments to methods for identifying unbiased molecular patterns that define clinical subsets of clear cell renal cell carcinoma (ccRCC). The presently disclosed subject matter also relates in some embodiments to methods for employing classification schema based at least in part on gene expression patterns to predict clinical outcomes and/or survival in subjects having the different subsets of ccRCC.

### BACKGROUND

Clear cell renal cell carcinoma, ccRCC, afflicts upwards of 50,000 patients annually (American Cancer Society, Inc., 2009). Most patients present initially with localized disease, managed with surgery, but, unfortunately, nearly a third develop recurrence and succumb to their disease. ccRCC incidence has increased uniformly over the last 30 years, associated with stage migration toward lower stages, likely due to the increased detection of lesions incidentally. However, there has not been commensurate improvement in survival. ccRCC tumors have variable natural histories, and genetic strategies have been largely unhelpful in identifying patients with higher or lower risk for recurrence due to the overwhelming association of this cancer with von Hippel-Lindau (VHL) tumor suppressor gene inactivation (Bank et al., 2006; Nickerson et al., 2008).

The Fuhrman classification system stratifies ccRCC by tumor cell morphology: low grade (grade 1), intermediate grades (grades 2 and 3), and high grade (grade 4) tumors, with corresponding association with RCC-related death (Frank et al., 2002). Prognostic scoring systems such as the UCLA Integrated Staging System (UISS) have been developed using these morphologic characteristics, tumor size, and patient performance status as well as the inherent characteristics of stage and nodal status (Zisman et al., 2001; Lam et al., 2005). Other algorithms incorporate post-operative clinical information, but have limited discriminative ability for the abundant intermediate grade and intermediate stage tumors, and they fail to account for molecular distinctions in tumors (Sorbellini et al., 2005). The molecular basis of this diversity in clinical behavior remains unclear.

What are needed, then, are new methods and compositions for analyzing subjects with ccRCC, particularly so that more accurate prognoses can be made and more appropriate treatment modalities can be employed for subjects based on the specifics of their diseases.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides in some embodiments methods for generating prognostic signatures for subject with clear cell renal cell carcinoma (ccRCC). In some embodiments, the methods comprise determining expression levels for three or more genes listed in Table 7 in ccRCC cells obtained from the subject, wherein the determining provides a prognostic signature for the subject. In some embodiments, the methods comprise determining expression levels for at least 4, 5, 6, 7, 8 9, 10, or all 120 of the genes listed in Table 7 in ccRCC cells obtained from the subject. In some embodiments, the method comprise determining expression levels for each of FLT1, FZD1, GIPC2, MAP7, and NPR3 in ccRCC cells obtained from the subject.

In some embodiments, the presently disclosed methods further comprise comparing the prognostic signature determined to a standard. In some embodiments, the standard comprises a gene expression profile of the one or more genes obtained from ccA cells obtained from one or more subjects with ccRCC, an expression profile of the one or more genes obtained from ccB cells obtained from one or more subjects with ccRCC, or both. In some embodiments, the comparing comprises employing a Single Sample Predictor (SSP), Principal Component Analysis (PCA), consensus clustering, logical analysis of data (LAD) analyses, or a combination thereof. In some embodiments, the gene expression profile of the one or more genes obtained from ccA cells in the standard comprises a mean expression level for the one or more genes in the ccA cells, the expression profile of the one or more genes obtained from ccB cells, or both. In some embodiments, if the standard comprises both gene expression profiles, the mean expression levels are determined separately for the one or more genes in the ccA cells and the one or more genes in the ccB cells. In some embodiments, the standard comprises both gene expression profiles and the method further comprises assigning with the SSP, PCA, consensus clustering, and/or LAD analyses the prognostic signature to either the mean expression level for the three or more genes in the ccA cells or the mean expression level for the three or more genes in the ccB cells. In some embodiments, the assigning comprises employing a Spearman correlation. In some embodiments, the assigning step is performed by a suitably-programmed computer. In some embodiments, the subject is a human.

The presently disclosed subject matter also provides methods for assessing risk of an adverse outcome of a subject with clear cell renal cell carcinoma (ccRCC). In some embodiments, the methods comprise determining a mean expression level for three or more genes selected from among those genes listed in Table 7 in a biological sample comprising ccRCC cells obtained from subject; and comparing the expression levels determined to a standard. In some embodiments, the three or more genes are selected from among FLT1, FZD1, GIPC2, MAP7, and NPR3. In some embodiments, the subject is a human. In some embodiments, evidence of the expression level is obtained by a method comprising gene expression profiling. In some embodiments, the gene expression profiling method is a PCR-based method, a microarray based method, or an antibody-based method. In some embodiments, the expression levels are normalized relative to the expression levels of one or more reference genes. In some embodiments, the expression levels of at least four of the genes listed in Table 7. In some embodiments, the methods comprise determining the expression levels of at least five of the genes listed in Table 7. In some embodiments, the comparing comprises employing a Single Sample Predictor (SSP), Principal Component Analysis (PCA), consensus clustering, logical analysis of data (LAD) analyses, or a combination thereof, optionally performed by a suitably programmed computer. In some embodiments, the gene expression profile of the one or more genes obtained from ccA cells in the standard comprises a mean expression level for the one or more genes in the ccA cells, the expression profile of the one or more genes obtained from ccB cells, or both. In some embodiments, if the standard comprises both gene expression profiles, the mean expression levels are determined separately for the one or more genes in the ccA cells and the one or more genes in the ccB cells. In some embodiments, the standard comprises both gene expression profiles and the method further comprises assigning with the SSP, PCA, consensus clustering, and/or LAD analyses the prognostic signature to either the mean expression level for the three or more genes in the ccA cells or the mean expression level for the three or more genes in the ccB cells. In some embodiments, the assigning comprises employing a Spearman correlation, optionally performed by a suitably-programmed computer.

The presently disclosed subject matter also provides in some embodiments methods for predicting a clinical outcome of a treatment in a subject having clear cell renal cell carcinoma (ccRCC). In some embodiments, the methods comprise (a) determining the expression levels of three or more genes listed in Table 7, optionally three or more of FLT1, FZD1, GIPC2, MAP7, and NPR3 in a biological sample comprising ccRCC cells obtained from the ccRCC of the subject; and (b) comparing the expression levels determined to a standard, wherein the comparing is predictive of the clinical outcome of the treatment in the subject. In some embodiments, the clinical outcome is expressed in terms of Recurrence-Free Interval (RFI), Overall Survival (OS), Disease-Free Survival (DFS), or Distant Recurrence-Free Interval (DRFI). In some embodiments, the methods comprise determining the expression levels of at least four, at least five, or at least ten of the genes listed in Table 7. In some embodiments, the treatment is selected from among surgical resection, chemotherapy, molecular targeted therapy, immunotherapy, and combinations thereof. In some embodiments, the comparing comprises employing a Single Sample Predictor (SSP), Principal Component Analysis (PCA), consensus clustering, logical analysis of data (LAD) analyses, or a combination thereof, optionally performed by a suitably programmed computer. In some embodiments, the standard comprises a gene expression profile of the one or more genes obtained from ccA cells obtained from one or more subjects with ccA, an expression profile of the one or more genes obtained from ccB cells obtained from one or more subjects with ccB, or both. In some embodiments, the gene expression profile of the one or more genes obtained from ccA cells in the standard comprises a mean expression level for the one or more genes in the ccA cells, the expression profile of the one or more genes obtained from ccB cells, or both. In some embodiments, if the standard comprises both gene expression profiles, the mean expression levels are determined separately for the one or more genes in the ccA cells and the one or more genes in the ccB cells. In some embodiments, the standard comprises both gene expression profiles and the method further comprises assigning with the SSP, PCA, consensus clustering, and/or LAD analyses the prognostic signature to either the mean expression level for the three or more genes in the ccA cells or the mean expression level for the three or more genes in the ccB cells. In some embodiments, the assigning comprises employing a Spearman correlation, optionally performed by a suitably programmed computer. In some embodiments, the gene expression profile of the three or more genes obtained from ccA cells in the standard comprises a mean expression level for the three or more genes in the ccA cells, the expression profile of the three or more genes obtained from ccB cells, or both, and further wherein if the standard comprises both gene expression profiles, the mean expression levels are determined separately for the three or more genes in the ccA cells and the three or more genes in the ccB cells. In some embodiments, the subject is a human.

The presently disclosed subject matter also provides in some embodiments arrays comprising polynucleotides that hybridize specifically to at least three genes listed in Table 7 or comprising specific peptide or polypeptide gene products of at least three genes listed in Table 7. In some embodiments, each specific peptide or polypeptide gene product present on the array is present thereon in an amount, relative to each other specific peptide or polypeptide gene product that is present on the array, that is reflective of the expression level of its corresponding gene in clear cell renal cell carcinoma (ccRCC) cells obtained from a subject with ccRCC. In some embodiments, the specific peptide or polypeptide gene products are present on the array such that the array is interrogatable with at least one antibody that specifically binds to one of the specific peptide or polypeptide gene products. In some embodiments, the array comprises at least one polynucleotide or specific peptide or polypeptide gene product for each of FLT1, FZD1, GIPC2, MAP7, and NPR3.

Thus, it is an object of the presently disclosed subject matter to provide in some embodiments methods and compositions for employing classification schema based at least in part on gene expression patterns to predict clinical outcomes and/or survival in subjects having the different subsets of ccRCC.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIGS. 1A and 1B are each a flow chart diagram depicting the order of analyses. (A) Delineation of steps taken to identify ccRCC subtypes. (B) Diagram of analyses to characterize and validate identified subtypes.

FIGS. 2A-2D are consensus matrixes demonstrating the presence of two core clusters of intermediate grade ccRCC. Consensus matrix heatmaps demonstrate the presence of two clusters within all clear cell tumors (FIG. 2A) and invariance of the two ccRCC core clusters using k=2 (FIG. 2B), k=3 (FIG. 2C), and k=4 (FIG. 2D) cluster assignments for each cluster method. Lighter gray areas, which correspond to red coloring in the full color concensus matrices, identify the similarity between samples and display samples clustered together across the bootstrap analysis. The ccA and ccB clusters are identified at the tope of each of FIGS. 2A-2D.

FIGS. 3A-3G are pathway analyses of subtypes that shows that ccA and ccB are highly dissimilar. FIG. 3A is a heat map of the 6213 probes differentially expressed between ccA and ccB as determined by SAM analysis (FDR<0.000001). FIGS. 3B-3G are magnified heatmaps of the genes from FIG. 3A that populate the ccA (FIGS. 3B-3D) or ccB (FIGS. 3E-3G) overexpressed Molecular Signatures Database (MSigDB; part of the Gene Set Enrichment Analysis (GSEA) collection of the Broad Institute, Cambridge, Mass., United States of America; see also Subramanian et al. (2005) Proc Nat Acad Sci USA 102:15545-15550 and Mootha et al. (2003) Nat Genet 34:267-273) curated gene sets of Brentani angiogenesis (FIG. 3B), beta-oxidation (FIG. 3C), HSA00071 fatty acid metabolism (FIG. 3D), EMT up (FIG. 3E), TGFβ C4 up (FIG. 3F), and Wnt targets (FIG. 3G).

FIGS. 4A and 4B show that LAD probes separated ccA and ccB tumor clusters. FIG. 4A is a heat map of gene expression data for core arrays and 120 logical analysis of data (LAD) probes. These probes were selected using LAD and leave-one-out analysis from 1075 distinguishing probes with p-value <0.000001. FIG. 4B is a series of digital images of blots showing semi-quantitative reverse transcription PCR analyses that validate the ability of a subset of the LAD probes to clearly distinguish between ccA and ccB tumors.

FIG. 5 is a consensus matrix depicting validation of LAD probes in validation dataset showing the existence of two ccRCC clusters. A consensus matrix of 177 ccRCC tumors determined by 111 probes corresponding to the 120 LAD probes is depicted. Lighter gray areas, which correspond to ted areas ni the full color consensus matrix, identify samples clustered together across the bootstrap analysis. Two distinct clusters are visible, validating the ability of the LAD probe set to classify ccRCC tumors into ccA or ccB subtypes from other array platforms.

FIGS. 6A-6D are a series of plots demonstrating that classification of tumors from validation dataset by LAD prediction showed that subtypes have differing survival outcomes. 177 ccRCC tumors were individually assigned to ccA, ccB, or unclassified by LAD prediction analysis, and cancer specific (FIG. 6A) or overall survival (FIG. 6B) were calculated via Kaplan-Meier curves. The ccB subtype had a significantly decreased survival outcome compared to ccA, while unclassified tumors had an intermediate survival time (log rank p<0.01). FIG. 6C is a plot of cancer specific survival for intermediate (Fuhrman grade 2-3) tumors that shows significant difference between subtypes. FIG. 6D is a plot of cancer specific survival for high grade (Fuhrman grade 4) that shows a trend of better survival for ccA tumors.

FIGS. 7A and 7B are a consensus matrix and a PCA plot, respectively, showing that two ccRCC subtypes are distinct from normal kidney tissue. Both consensus matrix (FIG. 7A) and the PCA plot (FIG. 7B; scatter plot of the top 2 eigenvectors—PC1, PC2) show the complete delineation between the clear cell tumors and corresponding normal kidney tissue removed from ccRCC patients. Red areas identify samples clustered together across the bootstrap analysis. These results verified that the subtypes did not arise from errors in the expression levels due to contamination from normal tissue.

FIGS. 8A-8F are a series of gel photographs depicting semi-quantitative reverse transcription PCR of FLT1 (FIG. 8A), FZD1 (FIG. 8B), GIPC2 (FIG. 8C), MAP7 (FIG. 8D), NPR3 (FIG. 8E), and an 18S rRNA control (FIG. 8F). These results validated the ability of a subset of the LAD probes to clearly distinguish between ccA and ccB tumors.

### BRIEF DESCRIPTION OF THE SEQUENCE LISTING

SEQ ID NOs: 1 and 2 are exemplary nucleotide and amino acid sequences, respectively, for human FLT1 gene products that correspond to GENBANK® Accession Nos. NM_{—}001159920 (nucleotide sequences) and NP_{—}001153392 (amino acid sequence).

SEQ ID NOs: 3 and 4 are exemplary nucleotide and amino acid sequences, respectively, for human FZD1 gene products that correspond to GENBANK® Accession Nos. NM_{—}003505 (nucleotide sequence) and NP_{—}003496 (amino acid sequence).

SEQ ID NOs: 5 and 6 are exemplary nucleotide and amino acid sequences, respectively, for human GIPC2 gene products that correspond to GENBANK® Accession Nos. NM_{—}017655 (nucleotide sequence) and NP_{—}060125 (amino acid sequence).

SEQ ID NOs: 7 and 8 are exemplary nucleotide and amino acid sequences, respectively, for human MAP7 gene products that correspond to GENBANK® Accession Nos. NM_{—}003980 (nucleotide sequence) and NP_{—}003971 (amino acid sequence).

SEQ ID NOs: 9 and 10 are exemplary nucleotide and amino acid sequences, respectively, for human NPR3 gene products that correspond to GENBANK® Accession Nos. NM_{—}000908 (nucleotide sequence) and NP_{—}000899 (amino acid sequence).

SEQ ID NOs: 11-20 are nucleotide sequences for exemplary oligonucleotides that can be employed for assaying expression levels of FLT1 (SEQ ID NOs: 11 and 12), FZD1 (SEQ ID NOs: 13 and 14), GIPC2 (SEQ ID NOs: 15 and 16), MAP7 (SEQ ID NOs: 17 and 18), and NPR3 (SEQ ID NOs: 19 and 20).

Each of the sequences listed the Tables, including the annotations and references cited in the corresponding database accession numbers (including, but not limited to the GENBANK® database), is incorporated herein by reference in its entirety.

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### I. GENERAL CONSIDERATIONS

Disclosed herein are methods that can be employed to focus on genes and/or pathways that are biologically relevant to kidney cancer and to identify and study those that can be of prognostic significance. By comparing ccA versus ccB tumors (optionally using a suitably programmed computer), molecular changes reflective of differences in biology within otherwise indistinguishable primary kidney tumors could be determined. The data presented herein show that there are distinct molecular changes in patients with ccA and ccB tumors, and that these alterations can be exploited for the study of novel targets. The prognostic value of these gene expression differences has also been evaluated, and the presently disclosed subject matter shows that they retain their prognostic value in multiple independent datasets. The prognostic signature can therefore be used to define patients most likely to benefit from surgery or chemotherapy and stratify patients in future clinical trials.

### II. DEFINITIONS

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “a cell” refers to one or more cells, unless the context clearly indicates otherwise.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein.

Similarly, all genes, gene names, and gene products disclosed herein are intended to correspond to orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

The term “about”, as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, an array can “consist essentially of” a specific number of locations that contain polynucleotides that are designed to hybridize to gene products encoded by and/or transcribed from one or more of the genes identified in Table 7, which means that the recited locations are the only locations present on the array that are designed to assay differential gene expression in a biological sample. It is noted, however, that additional locations on the array can include polynucleotides that are designed to act as positive or negative controls, as these are not designed to assay differential gene expression but are present to validate the effectiveness of the array and/or for producing data that can be compared across different independent experiments.

With respect to the terms “comprising”, “consisting essentially of”, and “consisting of”, where one of these three terms is used herein, the presently disclosed and claimed subject matter can include the use of either of the other two terms. For example, the presently disclosed subject matter relates in some embodiments to arrays for assaying gene expression in a biological sample comprising polynucleotides that hybridize to at least three genes selected from among those set forth in Table 7 and/or specific peptide or polypeptide gene products of at least three of the genes listed in Table 7. It is understood that the presently disclosed subject matter thus also encompasses arrays that in some embodiments consist essentially of polynucleotides that hybridize to at least three genes selected from among those set forth in Table 7 and/or specific peptide or polypeptide gene products of at least three of the genes listed in Table 7, as well as arrays that in some embodiments consist of polynucleotides that hybridize to at least three genes selected from among those set forth in Table 7 and/or specific peptide or polypeptide gene products of at least three of the genes listed in Table 7. Similarly, it is also understood that in some embodiments the methods of the presently disclosed subject matter comprise the steps that are disclosed herein, in some embodiments the methods of the presently disclosed subject matter consist essentially of the steps that are disclosed, and in some embodiments the methods of the presently disclosed subject matter consist of the steps that are disclosed herein.

As used herein, the terms “ccA” and “ccB” refer to clear cell type A (ccA) and clear cell type B (ccB), respectively, which are classifications of clear cell renal cell carcinoma (ccRCC) that can be made on the basis of the gene expression profiles disclosed herein. It is noted that while ccA and ccB cannot currently be distinguished morphologically, the gene expression profiles disclosed herein including, but not limited to gene expression analysis of three or more of the genes identified in Table 7 below, can be used to categorize a subject's ccRCC as either ccA or ccB. While the present disclosure exemplified the methods and compositions of the presently disclosed subject matter with the human genes FLT1, FZD1, GIPC2, MAP7, and NPR3, it is understood that all of the genes disclosed in Table 7 can be employed in any combination or subcombination of at least three of the genes disclosed therein. Thus, in some embodiments, the methods and compositions of the presently disclosed subject matter employ at least 3, 4, 5, 6, 7, 8, 9, 10, 20, 25, 50, 75, 100, or all 120 of the genes listed in Table 7 including every whole number between 3 and 120 inclusive.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well know to those of skill in the art, and these peptide fragments are also considered gene products.

It is understood that while the nucleotide and amino acid sequences disclosed herein are for human orthologs of various genes and gene products relevant to kidney cancer, orthologs of these genes and gene products from other species are also included within the presently disclosed subject matter.

As used herein, the term “FLT1” refers to the Fms-related tyrosine kinase 1 (vascular endothelial growth factor/vascular permeability factor receptor) gene. Exemplary FLT1 gene products are described in GENBANK® Accession Nos. CR593388 and NM_{—}001159920 (nucleotide sequences) and NP_{—}001153392 (amino acid sequence encoded thereby).

As used herein, the term “FZD1” refers to the Frizzled homolog 1 (Drosophila) gene. Exemplary FZD1 gene products are described in GENBANK® Accession Nos. NM_{—}003505 (nucleotide sequence) and NP_{—}003496 (amino acid sequence encoded thereby).

As used herein, the term “GIPC2” refers to the PDZ domain protein GIPC2 gene. Exemplary GIPC2 gene products are described in GENBANK® Accession Nos. NM_{—}017655 (nucleotide sequence) and NP_{—}060125 (amino acid sequence encoded thereby).

As used herein, the term “MAP7” refers to the Microtubule-associated protein 7 gene. Exemplary MAP7 gene products are described in GENBANK®Accession Nos. NM_{—}003980 (nucleotide sequence) and NP_{—}003971 (amino acid sequence encoded thereby).

As used herein, the term “NPR3” refers to the Natriuretic peptide receptor C/guanylate cyclase C (atrionatriuretic peptide receptor C) gene. Exemplary NPR3 gene products are described in GENBANK® Accession Nos. NM_{—}000908

(nucleotide sequence) and NP_{—}000899 (amino acid sequence encoded thereby).

The term “isolated”, as used in the context of a nucleic acid or polypeptide (including, for example, a peptide), indicates that the nucleic acid or polypeptide exists apart from its native environment. An isolated nucleic acid or polypeptide can exist in a purified form or can exist in a non-native environment. In some embodiments, “isolated” refers to a physical isolation, meaning that the cell, nucleic acid or peptide has been removed from its native environment (e.g., from a subject).

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, and “mRNA”. Nucleic acids can be synthesized, or can be derived from any biological source, including any organism.

As used herein, the terms “peptide” and “polypeptide” refer to polymers of at least two amino acids linked by peptide bonds. Typically, “peptides” are shorter than “polypeptides”, but unless the context specifically requires, these terms are used interchangeably herein.

As used herein, a cell, nucleic acid, or peptide exists in a “purified form” when it has been isolated away from some, most, or all components that are present in its native environment, but also when the proportion of that cell, nucleic acid, or peptide in a preparation is greater than would be found in its native environment. As such, “purified” can refer to cells, nucleic acids, and peptides that are free of all components with which they are naturally found in a subject, or are free from just a proportion thereof.

### III. METHODS FOR GENERATING PROGNOSTIC SIGNATURES

In some embodiments, the presently disclosed subject matter provides methods for generating prognostic signatures for a subject with kidney cancer (such as, but not limited to, kidney cancer of type ccA or of type ccB as defined herein). As used herein, the phrase “prognostic signature” refers to a gene expression profile comprising gene expression levels for three, four, five, six, seven, eight, nine, ten, or more of the genes disclosed in Table 7 below (such as, but not limited to, FLT1, FZD1, GIPC2, MAP7, and/or NPR3) in cancer cells obtained from the subject, wherein the determining provides a prognostic signature for the subject. As disclosed herein, when compared to appropriate standards, such gene expression profiles can be predictive of various clinical outcomes.

As used herein, the phrase “gene expression profiling” refers to examining expression of one or more RNAs in a cell, which in some embodiments involves examining mRNA expression levels in a cell. In some embodiments, at least or up to 10, 100, 100, 10,000, or more different mRNAs can be examined in a single experiment. In some embodiments, differential profiling (comparison with another cell; e.g., that has a different phenotype, e.g., normal vs. cancerous, normal vs. ccA, normal vs. ccB, ccA vs. ccB, etc.) provides useful information about the cell of interest (e.g., genes that are preferentially or selectively expressed in a ccA cell vs. a ccB cell, and/or genes that are over- or underexpressed in a ccA cell vs. a ccB cell). Thus, the results of gene expression profiling result in the generation of a “gene expression profile”, which includes a summary of the expression levels of some or all genes examined (in some embodiments, a summary of the expression levels of some or all of the genes listed in Table 7) in a given cell or group of cells (e.g., normal cells, ccA cells, or ccB cells) that can be compared to the gene expression profile of another given cell or group of cells (e.g., normal vs. cancerous, normal vs. ccA, normal vs. ccB, ccA vs. ccB, etc.).

Methods for examining gene expression, often but not always hybridization based, include, but are not limited to northern blots; dot blots; primer extension; nuclease protection; subtractive hybridization and isolation of non-duplexed molecules using, for example, hydroxyapatite; solution hybridization; filter hybridization; amplification techniques such as RT-PCR and other PCR-related techniques such as differential display, ligase chain reaction (LCR), amplified fragment length polymorphism (AFLP), etc. (see e.g., U.S. Pat. Nos. 4,683,195 and 4,683,202; Innis et al., 1990; Liang & Pardee, 1992; Hubank & Schatz, 1994; Perucho et al., 1995), fingerprinting, for example, with restriction endonucleases (Ivanova et al., 1995; Kato, 1995; and Shimkets et al., 1999; see also U.S. Pat. No. 5,871,697)); and the use of structure specific endonucleases (see e.g., De Francesco, 1998). mRNA expression can also be analyzed using mass spectrometry techniques (e.g., MALDI or SELDI), liquid chromatography, and capillary gel electrophoresis. For a general description of these techniques, see also Sambrook & Russell, 2001; Kriegler, 1990; and Ausubel et al., 2003.

Techniques have been developed that expedite expression analysis and sequencing of large numbers of nucleic acids samples. For example, nucleic acid arrays have been developed for high density and high throughput expression analysis (see e.g., Granjeuad et al., 1999; Lockhart & Winzeler, 2000). Nucleic acid arrays refer to large numbers (e.g., hundreds, thousands, tens of thousands, or more) of nucleic acid probes bound to solid substrates, such as nylon, glass, or silicon wafers (see e.g., Fodor et al., 1991; Brown & Botstein, 1999; Eberwine, 1996). A single array can contain, e.g., probes corresponding to an entire genome, or to all genes expressed by the genome. The probes on the array can be DNA oligonucleotide arrays (e.g., GENECHIP™, see e.g., Lipshutz et al., 1999), mRNA arrays, cDNA arrays, EST arrays, or optically encoded arrays on fiber optic bundles (e.g., BEADARRAY™). The samples applied to the arrays for expression analysis can be, e.g., PCR products, cDNA, mRNA, etc.

Additional techniques for rapid gene sequencing and analysis of gene expression include, for example, serial analysis of gene expression (SAGE). For SAGE, a short sequence tag (typically about 10-14 bp) contains sufficient information to uniquely identify a transcript. These sequence tags can be linked together to form long serial molecules that can be cloned and sequenced. Quantitation of the number of times a particular tag is observed proves the expression level of the corresponding transcript (see e.g., Velculescu et al., 1995; Velculescu et al., 1997; and de Waard et al., 1999).

In some embodiments, the methods for generating prognostic signatures further comprise comparing the derived prognostic signatures to one or more standards. As used herein, the term “standard” refers to an entity to which another entity (e.g., a prognostic signature) can be compared such that the comparison provides information of interest. An exemplary standard that is described herein is a test set. Additional discussion of standards can be found hereinbelow. In some embodiments, the comparing step is performed by a suitably programmed computer.

Thus, a profile can be created once an expression level is determined for a gene. As used herein, the term “profile” (e.g., a “gene expression profile”) refers to a repository of the expression level data that can be used to compare the expression levels of different genes among various subjects. For example, for a given subject, the term “profile” can encompass the expression levels of one or more of the genes disclosed herein detected in whatever units are chosen. The term “profile” is also intended to encompass manipulations of the expression level data derived from a subject. For example, once relative expression levels are determined for a given set of genes in a subject, the relative expression levels for that subject can be compared to a standard to determine if the expression levels in that subject are higher or lower than for the same genes in the standard. Standards can include any data deemed to be relevant for comparison.

### IV. METHODS FOR ASSESSING RISKS OF ADVERSE OUTCOMES

The presently disclosed subject matter also provides methods for assessing risk of an adverse outcome of a subject with kidney cancer.

In some embodiments, the methods comprise determining an expression level for three or more genes selected from among those set forth in Table 7 below (e.g., FLT1, FZD1, GIPC2, MAP7, and/or NPR3) in a biological sample comprising kidney cancer cells obtained from subject; and comparing the expression levels determined to a standard. In some embodiments, the comparing step is indicative of an increased likelihood that an adverse outcome (including, but not limited to decreased Overall Survival (OS) and/or Disease-Free Survival (DFS)) would occur in a subject relative to other subjects with kidney cancer. In some embodiments, the comparing step is performed by a suitably programmed computer.

### V. METHODS FOR PREDICTING CLINICAL OUTCOMES FROM TREATMENTS

The presently disclosed subject matter also provides methods for predicting a clinical outcome of a treatment in a subject diagnosed with kidney cancer. In some embodiments, the methods comprise (a) determining the expression level of three or more genes selected from among those set forth in Table 7 (such as, but not limited to FLT1, FZD1, GIPC2, MAP7, and/or NPR3) in a biological sample comprising cancer cells obtained from the kidney of the subject; and (b) comparing the expression levels determined to a standard, wherein the comparing is predictive of the clinical outcome of the treatment in the subject. In some embodiments, the comparing step is performed by a suitably programmed computer.

As used herein, the phrase “clinical outcome” refers to any measure by which a treatment designed to treat kidney cancer can be measured. Exemplary clinical outcomes include Recurrence-Free Interval (RFI), Overall Survival (OS), Disease-Free Survival (DFS), or Distant Recurrence-Free Interval (DRFI).

### VI. METHODS FOR PREDICTING A POSITIVE OR A NEGATIVE CLINICAL RESPONSE IN A SUBJECT

The presently disclosed subject matter also provides methods for predicting a positive or a negative clinical response of a subject with kidney cancer to a treatment such as, but not limited to treatment with targeted therapeutics, immunological agents, biological agents, chemotherapy, radiotherapy, and combinations thereof. In some embodiments, the treatment can comprise IL-2 therapy, vascular endothelial growth factor (VEGF) and/or

VEGF pathway targeted therapy, and/or mammalian target of rapamycin (mTOR) directed therapy. It is understood, however, that the compositions and methods of the presently disclosed subject matter can be employed for predicting a positive or a negative clinical response of a subject with kidney cancer to any treatment modality including, but not limited to those expressly described herein.

In some embodiments, the methods comprise (a) determining the expression levels of at least three genes selected from among those set forth in Table 7 (such as, but not limited to FLT1, FZD1, GIPC2, MAP7, and/or NPR3) in a biological sample comprising cancer cells obtained from the kidney of the subject; and (b) comparing the expression levels determined to a first expression profile and a second expression profile, wherein (i) the first expression profile is generated by determining the expression levels of the same genes in kidney cancer cells obtained from one or more subjects with ccA; (ii) the second expression profile is generated by determining the expression levels of the same genes in kidney cancer cells obtained from one or more subjects with ccB; and (iii) assigning the expression levels determined for the at least three genes in the biological sample obtained from the subject to either the first expression profile or the second expression profile, and further wherein assigning the expression levels determined for the genes in the biological sample obtained from the subject to the first expression profile is indicative of a positive clinical response and assigning the expression levels determined for the at least five genes in the biological sample obtained from the subject to the second expression profile is indicative of a negative clinical response. In some embodiments, the first, the second, or both the first and second expression levels are mean expression levels. In some embodiments, the comparing step, the assigning step, or both is/are performed by a suitably programmed computer.

### VII. METHODS OF GENE EXPRESSION ANALYSIS

VII.A. Nucleic Acid Assay Formats

The genes identified as being differentially expressed in ccA versus ccB type kidney cancer can be used in a variety of nucleic acid detection assays to detect or quantitate the expression level of a gene or multiple genes in a given sample. For example, Northern blotting, nuclease protection, RT-PCR (e.g., quantitative RT-PCR; QRT-PCR), and/or differential display methods can be used for detecting gene expression levels. In some embodiments, methods and assays of the presently disclosed subject matter are employed with array or chip hybridization-based methods for detecting the expression of a plurality of genes.

Any hybridization assay format can be used, including solution-based and solid support-based assay formats. Representative solid supports containing oligonucleotide probes for differentially expressed genes of the presently disclosed subject matter can be filters, polyvinyl chloride dishes, silicon, glass based chips, etc. Such wafers and hybridization methods are widely available and include, for example, those disclosed in PCT International Patent Application Publication WO 95/11755). Any solid surface to which oligonucleotides can be bound, either directly or indirectly, either covalently or non-covalently, can be used. An exemplary solid support is a high-density array or DNA chip. These contain a particular oligonucleotide probe in a predetermined location on the array. Each predetermined location can contain more than one molecule of the probe, but in some embodiments each molecule within the predetermined location has an identical sequence. Such predetermined locations are termed features. There can be any number of features on a single solid support including, for example, about 2, 10, 100, 1000, 10,000, 100,000, or 400,000 of such features on a single solid support. The solid support, or the area within which the probes are attached, can be of any convenient size (for example, on the order of a square centimeter).

Oligonucleotide probe arrays for differential gene expression monitoring can be made and employed according to any techniques known in the art (see e.g., Lockhart et al., 1996; McGall et al., 1996). Such probe arrays can contain at least two or more oligonucleotides that are complementary to or hybridize to two or more of the genes described herein. Such arrays can also contain oligonucleotides that are complementary or hybridize to at least about 2, 3, 4, 5, 6, 7, 8, 9, 10, 15, 20, 25, 30, 50, 70, 100, or more of the nucleic acid sequences disclosed herein.

The genes that are assayed according to the presently disclosed subject matter are typically in the form of RNA (e.g., total RNA or mRNA) or reverse transcribed RNA. The genes can be cloned or not, and the genes can be amplified or not. In some embodiments, poly A⁺ RNA is employed as a source.

Probes based on the sequences of the genes described herein can be prepared by any commonly available method. Oligonucleotide probes for assaying the tissue or cell sample are in some embodiments of sufficient length to specifically hybridize only to appropriate complementary genes or transcripts. Typically, the oligonucleotide probes are at least 10, 12, 14, 16, 18, 20, or 25 nucleotides in length. In some embodiments, longer probes of at least 30, 40, 50, or 60 nucleotides are employed.

As used herein, oligonucleotide sequences that are complementary to one or more of the genes described herein are oligonucleotides that are capable of hybridizing under stringent conditions to at least part of the nucleotide sequence of said genes. Such hybridizable oligonucleotides will typically exhibit in some embodiments at least about 75% sequence identity, in some embodiments about 80% sequence identity, in some embodiments about 85% sequence identity, in some embodiments about 90% sequence identity, in some embodiments about 91% sequence identity, in some embodiments about 92% sequence identity, in some embodiments about 93% sequence identity, in some embodiments about 94% sequence identity, in some embodiments about 95% sequence identity, and in some embodiments greater than 95% sequence identity (e.g., 96%, 97%, 98%, 99%, or 100% sequence identity) at the nucleotide level to the nucleic acid sequences disclosed herein and/or the reverse complements thereof.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals can also be produced by intrinsic fluorescence of the array components themselves. A single background signal can be calculated for the entire array, or a different background signal can be calculated for each target nucleic acid. In some embodiments, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack probes.

Assays and methods of the presently disclosed subject matter can utilize available formats to simultaneously screen in some embodiments at least about 10, in some embodiments at least about 50, in some embodiments at least about 100, in some embodiments at least about 1000, in some embodiments at least about 10,000, and in some embodiments at least about 40,000 or more different nucleic acid hybridizations.

The terms “mismatch control” and “mismatch probe” refer to a probe comprising a sequence that is deliberately selected not to be perfectly complementary to a particular target sequence. For each mismatch (MM) control in a high-density array there typically exists a corresponding perfect match (PM) probe that is perfectly complementary to the same particular target sequence. The mismatch can comprise one or more bases.

While the mismatch(s) can be located anywhere in the mismatch probe, terminal mismatches are less desirable as a terminal mismatch is less likely to prevent hybridization of the target sequence. In some embodiments, the mismatch is located at or near the center of the probe such that the mismatch is most likely to destabilize the duplex with the target sequence under the test hybridization conditions.

The phrase “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe, or the like. A perfect match control or perfect match probe is, however, distinguished from a “mismatch control” or “mismatch probe”.

As used herein, a “probe” is defined as a nucleic acid that is capable of binding to a target nucleic acid of complementary sequence through one or more types of chemical bonds, usually through complementary base pairing, usually through hydrogen bond formation. As used herein, a probe can include natural (i.e., A, G, U, C, or T) or modified bases (7-deazaguanosine, inosine, etc.). In addition, the bases in probes can be joined by a linkage other than a phosphodiester bond, so long as it does not interfere with hybridization. Thus, probes can be peptide nucleic acids in which the constituent bases are joined by peptide bonds rather than phosphodiester linkages.

VII.A1. Probe Design

Upon review of the present disclosure, one of skill in the art will appreciate that an enormous number of array designs are suitable for the practice of the presently disclosed subject matter. The high-density array typically includes a number of probes that specifically hybridize to the sequences of interest. See PCT International Patent Application Publication WO 99/32660, incorporated herein be reference in its entirety, for methods of producing probes for a given gene or genes. In addition, in some embodiments, the array includes one or more control probes.

High-density array chips of the presently disclosed subject matter include in some embodiments “test probes”. Test probes can be oligonucleotides that in some embodiments range from about 5 to about 500 or about 5 to about 50 nucleotides, in some embodiments from about 10 to about 40 nucleotides, and in some embodiments from about 15 to about 40 nucleotides in length. In some embodiments, the probes are about 20 to 25 nucleotides in length. In some embodiments, test probes are double or single strand DNA sequences. DNA sequences are isolated or cloned from natural sources and/or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high-density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that can cause the signal of a perfect hybridization to vary between arrays. In some embodiments, signals (e.g., fluorescence intensity) read from some or all other probes in the array are divided by the signal (e.g., fluorescence intensity) from the control probes, thereby normalizing the measurements.

Virtually any probe can serve as a normalization control. However, it is recognized that hybridization efficiency varies with base composition and probe length. Exemplary normalization probes can be selected to reflect the average length of the other probes present in the array; however, they can be selected to cover a range of lengths. The normalization control(s) can also be selected to reflect the (average) base composition of the other probes in the array; however, in some embodiments, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to, the β-actin gene, the transferrin receptor gene, the GAPDH gene, and the like.

Mismatch controls can also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). In some embodiments, mismatch probes contain one or more central mismatches. Thus, for example, where a probe is a 20-mer, a corresponding mismatch probe will have the identical sequence except for a single base mismatch (e.g., substituting a G, a C, or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a given hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (IBM)-I(MM)) provides a good measure of the concentration of the hybridized material.

VII.A.2. Nucleic Acid Samples

A biological sample that can be analyzed in accordance with the presently disclosed subject matter comprises in some embodiments a nucleic acid. The terms “nucleic acid”, “nucleic acids”, and “nucleic acid molecules” each refer in some embodiments to deoxyribonucleotides, ribonucleotides, and polymers and folded structures thereof in either single- or double-stranded form. Nucleic acids can be derived from any source, including any organism. Deoxyribonucleic acids can comprise genomic DNA, cDNA derived from ribonucleic acid, DNA from an organelle (e.g., mitochondrial DNA or chloroplast DNA), or combinations thereof. Ribonucleic acids can comprise genomic RNA (e.g., viral genomic RNA), messenger RNA (mRNA), ribosomal RNA (rRNA), transfer RNA (tRNA), or combinations thereof.

VII.A.2.a. Isolation of Nucleic Acid Samples

Nucleic acid samples used in the methods and assays of the presently disclosed subject matter can be prepared by any available method or process. Methods of isolating total mRNA are also known to those of skill in the art. For example, methods of isolation and purification of nucleic acids are described in detail in Chapter 3 of Tijssen, 1993. Such samples include RNA samples, but also include cDNA synthesized from an mRNA sample isolated from a cell or tissue of interest. Such samples also include DNA amplified from the cDNA, an RNA transcribed from the amplified DNA, and combinations thereof. One of skill in the art would appreciate that it can be desirable to inhibit or destroy RNase present in homogenates before homogenates are used as a source of RNA.

The presently disclosed subject matter encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (Kohsaka & Carson, 1994; Millar et al., 1995), filtration columns (Bej et al., 1991), or immunomagnetic beads (Albert et al., 1992; Chiodi et al., 1992). Such approaches can significantly increase the sensitivity of subsequent detection methods.

As one example, SEPHADEX® matrix (Sigma of St. Louis, Mo., United States of America) is a matrix of diatomaceous earth and glass suspended in a solution of chaotropic agents and has been used to bind nucleic acid material (Boom et al., 1990; Buffone et al., 1991). After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses (Lanciotti et al., 1992).

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, poly A⁺ RNA, rRNA, tRNA) followed by optional combination of multiple nucleic acid types into a single sample.

When RNA (e.g., mRNA) is selected for analysis, the disclosed methods allow for an assessment of gene expression in the tissue or cell type from which the RNA was isolated. RNA isolation methods are known to one of skill in the art. See Albert et al., 1992; Busch et al., 1992; Hamel et al., 1995; Herrewegh et al., 1995; Izraeli et al., 1991; McCaustland et al., 1991; Natarajan et al., 1994; Rupp et al., 1988; Tanaka et al., 1994; and Vankerckhoven et al., 1994.

Simple and semi-automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith 1998; and Paladichuk 1999.

In some embodiments, nucleic acids that are used for subsequent amplification and labeling are analytically pure as determined by spectrophotometric measurements or by visual inspection following electrophoretic resolution. In some embodiments, the nucleic acid sample is free of contaminants such as polysaccharides, proteins, and inhibitors of enzyme reactions. When a biological sample comprises an RNA molecule that is intended for use in producing a probe, it is preferably free of DNase and RNase. Contaminants and inhibitors can be removed or substantially reduced using resins for DNA extraction (e.g., CHELEX™ 100 from BioRad Laboratories of Hercules, Calif., United States of America) or by standard phenol extraction and ethanol precipitation.

VII.A.2.b. Amplification of Nucleic Acid Samples

In some embodiments, a nucleic acid isolated from a biological sample is amplified prior to being used in the methods disclosed herein. In some embodiments, the nucleic acid is an RNA molecule, which is converted to a complementary DNA (cDNA) prior to amplification. Techniques for the isolation of RNA molecules and the production of cDNA molecules from the RNA molecules are known (see generally, Silhavy et al., 1984; Sambrook & Russell, 2001; Ausubel et al., 2002; and Ausubel et al., 2003). In some embodiments, the amplification of RNA molecules isolated from a biological sample is a quantitative amplification (e.g., by quantitative RT-PCR).

The terms “template nucleic acid” and “target nucleic acid” as used herein each refer to nucleic acids isolated from a biological sample as described herein above. The terms “template nucleic acid pool”, “template pool”, “target nucleic acid pool”, and “target pool” each refer to an amplified sample of “template nucleic acid”. Thus, a target pool comprises amplicons generated by performing an amplification reaction using the template nucleic acid. In some embodiments, a target pool is amplified using a random amplification procedure as described herein.

The term “target-specific primer” refers to a primer that hybridizes selectively and predictably to a target sequence, for example a subsequence of one of the six genes disclosed herein, in a target nucleic acid sample. A target-specific primer can be selected or synthesized to be complementary to known nucleotide sequences of target nucleic acids.

The term “random primer” refers to a primer having an arbitrary sequence. The nucleotide sequence of a random primer can be known, although such sequence is considered arbitrary in that it is not specifically designed for complementarity to a nucleotide sequence of the presently disclosed subject matter. The term “random primer” encompasses selection of an arbitrary sequence having increased probability to be efficiently utilized in an amplification reaction. For example, the Random Oligonucleotide Construction Kit (ROCK) is a macro-based program that facilitates the generation and analysis of random oligonucleotide primers (Strain & Chmielewski, 2001). Representative primers include but are not limited to random hexamers and rapid amplification of polymorphic DNA (RAPD)-type primers as described by Williams et al., 1990.

A random primer can also be degenerate or partially degenerate as described by Telenius et al., 1992. Briefly, degeneracy can be introduced by selection of alternate oligonucleotide sequences that can encode a same amino acid sequence.

In some embodiments, random primers can be prepared by shearing or digesting a portion of the template nucleic acid sample. Random primers so-constructed comprise a sample-specific set of random primers.

The term “heterologous primer” refers to a primer complementary to a sequence that has been introduced into the template nucleic acid pool. For example, a primer that is complementary to a linker or adaptor, as described below, is a heterologous primer. Representative heterologous primers can optionally include a poly(dT) primer, a poly(T) primer, or as appropriate, a poly(dA) or poly(A) primer.

The term “primer” as used herein refers to a contiguous sequence comprising in some embodiments about 6 or more nucleotides, in some embodiments about 10-20 nucleotides (e.g., 15-mer), and in some embodiments about 20-30 nucleotides (e.g., a 22-mer). Primers used to perform the methods of the presently disclosed subject matter encompass oligonucleotides of sufficient length and appropriate sequence so as to provide initiation of polymerization on a nucleic acid molecule.

U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods of the presently disclosed subject matter, any PCR technique or related technique can be employed to perform the step of amplifying the nucleic acid sample. In addition, such methods can be optimized for amplification of a particular subset of nucleic acid (e.g., genomic DNA versus RNA), and representative optimization criteria and related guidance can be found in the art. See Cha & Thilly, 1993; Linz et al., 1990; Robertson & Walsh-Weller, 1998; Roux 1995; Williams 1989; and McPherson et al., 1995.

VII.A.3. Labeling of Nucleic Acid Samples

Optionally, a nucleic acid sample (e.g., a quantitatively amplified RNA sample) further comprises a detectable label. In some embodiments of the presently disclosed subject matter, the amplified nucleic acids can be labeled prior to hybridization to an array. Alternatively, randomly amplified nucleic acids are hybridized with a set of probes, without prior labeling of the amplified nucleic acids. For example, an unlabeled nucleic acid in the biological sample can be detected by hybridization to a labeled probe. In some embodiments, both the randomly amplified nucleic acids and the one or more pathogen-specific probes include a label, wherein the proximity of the labels following hybridization enables detection. An exemplary procedure using nucleic acids labeled with chromophores and fluorophores to generate detectable photonic structures is described in U.S. Pat. No. 6,162,603 to Heller.

In accordance with the methods of the presently disclosed subject matter, the amplified nucleic acids and/or probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to FITC (fluorescein isothiocyanate), FLUOR X™, ALEXA FLUOR® 488, OREGON GREEN® 488, 6-JOE (6-carboxy-4′,5′-dichloro-2′,7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech of Piscataway, N.J., United States of America or from Molecular Probes Inc. of Eugene, Oreg., United States of America). Fluorescent tags also include sulfonated cyanine dyes (available from Li-Cor, Inc. of Lincoln, Nebr., United States of America) that can be detected using infrared imaging. Methods for direct labeling of a heterogeneous nucleic acid sample are known in the art and representative protocols can be found in, for example, DeRisi et al., 1996; Sapolsky & Lipshutz, 1996; Schena et al., 1995; Schena et al., 1996; Shalon et al., 1996; Shoemaker et al., 1996; and Wang et al., 1998.

In some embodiments, nucleic acid molecules isolated from different cell types (e.g., ccA cells versus ccB cells) are labeled with different detectable markers, allowing the nucleic acids to be analyzed simultaneously on an array. For example, a first RNA sample can be reverse transcribed into cDNAs labeled with cyanine 3 (a green dye fluorophore; Cy3) while a second RNA sample to which the first RNA sample is to be compared can be labeled with cyanine 5 (a red dye fluorophore; Cy5).

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner, 1995). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al., 2000. Thus, it will be understood to one of skill in the art that labeling methods can be optimized for performance in microarray hybridization assay, and that optimal labeling can be unique to each label type.

VII.A.4. Forming High-Density Arrays

In some embodiments of the presently disclosed subject matter, probes or probe sets are immobilized on a solid support such that a position on the support identifies a particular probe or probe set. In the case of a probe set, constituent probes of the probe set can be combined prior to placement on the solid support or by serial placement of constituent probes at a same position on the solid support.

A microarray can be assembled using any suitable method known to one of skill in the art, and any one microarray configuration or method of construction is not considered to be a limitation of the presently disclosed subject matter. Representative microarray formats that can be used in accordance with the methods of the presently disclosed subject matter are described herein below and include, but are not limited to light-directed chemical coupling, and mechanically directed coupling (see U.S. Pat. Nos. 5,143,854 to Pirrunq et al.; 5,800,992 to Fodor et al.; and 5,837,832 to Chee et al.).

VII.A.4.a. Array Substrate and Configuration

The substrate for printing the array should be substantially rigid and amenable to DNA immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include but are not limited to a glass microscope slide, a glass coverslip, silicon, plastic, a polymer matrix, an agar gel, a polyacrylamide gel, and a membrane, such as a nylon, nitrocellulose, or ANAPORE™ (Whatman of Maidstone, United Kingdom) membrane.

Porous substrates (membranes and polymer matrices) are preferred in that they permit immobilization of relatively large amount of probe molecules and provide a three-dimensional hydrophilic environment for biomolecular interactions to occur (Dubiley et al., 1997; Yershov et al., 1996). A BIOCHIP ARRAYER™ dispenser (Packard Instrument Company of Meriden, Conn., United States of America) can effectively dispense probes onto membranes such that the spot size is consistent among spots whether one, two, or four droplets were dispensed per spot (Englert, 2000).

A microarray substrate for use in accordance with the methods of the presently disclosed subject matter can have either a two-dimensional (planar) or a three-dimensional (non-planar) configuration. An exemplary three-dimensional microarray is the FLOW-THRU™ chip (Gene Logic, Inc. of Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al., 1998.

Briefly, a FLOW-THRU™ chip (Gene Logic, Inc.) comprises a uniformly porous substrate having pores or microchannels connecting upper and lower faces of the chip. Probes are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767 to Beattie.

VII.A.4.b. Surface Chemistry

The particular surface chemistry employed is inherent in the microarray substrate and substrate preparation. Probe immobilization of nucleic acids probes post-synthesis can be accomplished by various approaches, including adsorption, entrapment, and covalent attachment. Typically, the binding technique is designed to not disrupt the activity of the probe.

For substantially permanent immobilization, covalent attachment is generally performed. Since few organic functional groups react with an activated silica surface, an intermediate layer is advisable for substantially permanent probe immobilization. Functionalized organosilanes can be used as such an intermediate layer on glass and silicon substrates (Liu & Hlady, 1996; Shriver-Lake 1998). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GM BS) that can bind maleimide to a primary amine of a probe. Procedures for using such linkers are known to one of skill in the art and are summarized by Hermanson 1990. A representative protocol for covalent attachment of DNA to silicon wafers is described by O'Donnell et al., 1997.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner, 1997; Schena et al., 1995). See also Worley et al., 2000.

For attachment of probes greater than about 300 base pairs, noncovalent binding is suitable. A representative technique for noncovalent linkage involves use of sodium isothiocyanate (NaSCN) in the spotting solution. When using this method, amino-silanized slides are typically employed because this coating improves nucleic acid binding when compared to bare glass. This method works well for spotting applications that use about 100 ng/μl (Worley et al., 2000).

In the case of nitrocellulose or nylon membranes, the chemistry of nucleic acid binding chemistry to these membranes has been well characterized (Southern, 1975; Sambrook & Russell, 2001).

VII.A.4.c. Arraying Techniques

A microarray for the detection of pathogens in a biological sample can be constructed using any one of several methods available in the art, including but not limited to photolithographic and microfluidic methods, further described herein below. In some embodiments, the method of construction is flexible, such that a microarray can be tailored for a particular purpose.

As is standard in the art, a technique for making a microarray should create consistent and reproducible spots. Each spot is preferably uniform, and appropriately spaced away from other spots within the configuration. A solid support for use in the presently disclosed subject matter comprises in some embodiments about 10 or more spots, in some embodiments about 100 or more spots, in some embodiments about 1,000 or more spots, and in some embodiments about 10,000 or more spots. In some embodiments, the volume deposited per spot is about 10 picoliters to about 10 nanoliters, and in some embodiments about 50 picoliters to about 500 picoliters. The diameter of a spot is in some embodiments about 50 μm to about 1000 μm, and in some embodiments about 100 μm to about 250 μm.

Light-Directed Synthesis.

This technique was developed by Fodor et al. (Fodor et al., 1991; Fodor et al., 1993), and commercialized by Affymetrix of Santa Clara, Calif., United States of America. Briefly, the technique uses precision photolithographic masks to define the positions at which single, specific nucleotides are added to growing single-stranded nucleic acid chains. Through a stepwise series of defined nucleotide additions and light-directed chemical linking steps, high-density arrays of defined oligonucleotides are synthesized on a solid substrate. A variation of the method, called Digital Optical Chemistry, employs mirrors to direct light synthesis in place of photolithographic masks (PCT International Patent Application Publication No. WO 99/63385). This approach is generally limited to probes of about 25 nucleotides in length or less. See also Warrington et al., 2000.

Contact Printing.

Several procedures and tools have been developed for printing microarrays using rigid pin tools. In surface contact printing, the pin tools are dipped into a sample solution, resulting in the transfer of a small volume of fluid onto the tip of the pins. Touching the pins or pin samples onto a microarray surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8×12 format, spaced at 9-mm centers that are compatible with 96- and 384-well plates. The pins are dipped into the wells, lifted, moved to a position over the microarray substrate, lowered to touch the solid support, whereby the sample is transferred. The process is repeated to complete transfer of all the samples. See Maier et al., 1994. A recent modification of solid pins involves the use of solid pin tips having concave bottoms, which print more efficiently than flat pins in some circumstances. See Rose, 2000.

Solid pins for microarray printing can be purchased, for example, from TeleChem International, Inc. of Sunnyvale, Calif. in a wide range of tip dimensions. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based array tools, including printing capillaries, tweezers, and split pins have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al., 1995. The diameter of the capillary typically ranges from about 10 μm to about 100 μm. A robot then moves the head with quills to the desired location for dispensing. The quill carries the sample to all spotting locations, where a fraction of the sample is deposited. The forces acting on the fluid held in the quill must be overcome for the fluid to be released. Accelerating and then decelerating by impacting the quill on a microarray substrate accomplishes fluid release. When the tip of the quill hits the solid support, the meniscus is extended beyond the tip and transferred onto the substrate. Carrying a large volume of sample fluid minimizes spotting variability between arrays. Because tapping on the surface is required for fluid transfer, a relatively rigid support, for example a glass slide, is appropriate for this method of sample delivery.

A variation of the pin printing process is the PIN-AND-RING™ technique developed by Genetic MicroSystems Inc. of Woburn, Mass., United States of America. This technique involves dipping a small ring into the sample well and removing it to capture liquid in the ring. A solid pin is then pushed through the sample in the ring, and the sample trapped on the flat end of the pin is deposited onto the surface. See Mace et al., 2000. The PIN-AND-RING™ technique is suitable for spotting onto rigid supports or soft substrates such as agar, gels, nitrocellulose, and nylon. A representative instrument that employs the PIN-AND-RING™ technique is the 417™ Arrayer available from Affymetrix of Santa Clara, Calif., United States of America.

Additional procedural considerations relevant to contact printing methods, including array layout options, print area, print head configurations, sample loading, preprinting, microarray surface properties, sample solution properties, pin velocity, pin washing, printing time, reproducibility, and printing throughput are known in the art, and are summarized by Rose, 2000.

Noncontact Ink-Jet Printing.

A representative method for noncontact ink-jet printing uses a piezoelectric crystal closely apposed to the fluid reservoir. One configuration places the piezoelectric crystal in contact with a glass capillary that holds the sample fluid. The sample is drawn up into the reservoir and the crystal is biased with a voltage, which causes the crystal to deform, squeeze the capillary, and eject a small amount of fluid from the tip. Piezoelectric pumps offer the capability of controllable, fast jetting rates and consistent volume deposition. Most piezoelectric pumps are unidirectional pumps that need to be directly connected, for example by flexible capillary tubing, to a source of sample supply or wash solution. The capillary and jet orifices should be of sufficient inner diameter so that molecules are not sheared. The void volume of fluid contained in the capillary typically ranges from about 100 μl to about 500 μl and generally is not recoverable. See U.S. Pat. No. 5,965,352 to Stoughton & Friend.

Devices that provide thermal pressure, sonic pressure, or oscillatory pressure on a liquid stream or surface can also be used for ink-jet printing. See Theriault et al., 1999.

Syringe-Solenoid Printing.

Syringe-solenoid technology combines a syringe pump with a microsolenoid valve to provide quantitative dispensing of nanoliter sample volumes. A high-resolution syringe pump is connected to both a high-speed microsolenoid valve and a reservoir through a switching valve. For printing microarrays, the system is filled with a system fluid, typically water, and the syringe is connected to the microsolenoid valve. Withdrawing the syringe causes the sample to move upward into the tip. The syringe then pressurizes the system such that opening the microsolenoid valve causes droplets to be ejected onto the surface. With this configuration, a minimum dispense volume is on the order of 4 nl to 8 nl. The positive displacement nature of the dispensing mechanism creates a substantially reliable system. See U.S. Pat. Nos. 5,743,960 and 5,916,524, both to Tisone.

Electronic Addressing.

This method involves placing charged molecules at specific positions on a blank microarray substrate, for example a NANOCHIP™ substrate (Nanogen Inc. of San Diego, Calif., United States of America). A nucleic acid probe is introduced to the microchip, and the negatively-charged probe moves to the selected charged position, where it is concentrated and bound. Serial application of different probes can be performed to assemble an array of probes at distinct positions. See U.S. Pat. No. 6,225,059 to Ackley et al. and PCT International Patent Application Publication No. WO 01/23082.

Nanoelectrode Synthesis.

An alternative array that can also be used in accordance with the methods of the presently disclosed subject matter provides ultra small structures (nanostructures) of a single or a few atomic layers synthesized on a semiconductor surface such as silicon. The nanostructures can be designed to correspond precisely to the three-dimensional shape and electro-chemical properties of molecules, and thus can be used to recognize nucleic acids of a particular nucleotide sequence. See U.S. Pat. No. 6,123,819 to Peeters.

In brief, the light-directed combinatorial synthesis of oligonucleotide arrays on a glass surface proceeds using automated phosphoramidite chemistry and chip masking techniques. In some embodiments, a glass surface is derivatized with a silane reagent containing a functional group, e.g., a hydroxyl or amine group blocked by a photolabile protecting group. Photolysis through a photolithogaphic mask is used selectively to expose functional groups that are then ready to react with incoming 5′ photoprotected nucleoside phosphoramidites. The phosphoramidites react only with those sites that are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, other methods that can be used to generate an array of oligonucleotides on a single substrate are described in PCT

International Patent Application Publication WO 93/09668. High-density nucleic acid arrays can also be fabricated by depositing pre-made and/or natural nucleic acids in predetermined positions. Synthesized or natural nucleic acids are deposited on specific locations of a substrate by light directed targeting and oligonucleotide directed targeting. A dispenser that moves from region to region to deposit nucleic acids in specific spots can also be employed.

VII.A.5. Hybridization

VII.A.5.a. General Considerations

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen, 1993. In general, a signal to noise ratio of 2-fold (or higher) than that observed for a negative control probe in a same hybridization assay indicates detection of specific or substantial hybridization.

VII.A.5.b. Hybridization on a Solid Support

In some embodiments of the presently disclosed subject matter, an amplified and/or labeled nucleic acid sample is hybridized to specific probes or probe sets that are immobilized on a continuous solid support comprising a plurality of identifying positions. Representative formats of such solid supports are described herein.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm ethylene diamine tetraacetic acid (EDTA), 1% BSA at 50° C. followed by washing in 2×SSC, 0.1% SDS at 50° C.; in another example, a probe and target sequence hybridize in 7% SDS, 0.5 M NaPO₄, 1 mm EDTA, 1% BSA at 50° C. followed by washing in 1×SSC, 0.1% SDS at 50° C.; in another example, a probe and target sequence hybridize in 7% SDS, 0.5 M NaPO₄, 1 mm EDTA, 1% BSA at 50° C. followed by washing in 0.5×SSC, 0.1% SDS at 50° C.; in another example, a probe and target sequence hybridize in 7% SDS, 0.5 M NaPO₄, 1 mm EDTA, 1% BSA at 50° C. followed by washing in 0.1×SSC, 0.1% SDS at 50° C.; in yet another example, a probe and target sequence hybridize in 7% SDS, 0.5 M NaPO₄, 1 mm EDTA, 1% BSA at 50° C. followed by washing in 0.1×SSC, 0.1% SDS at 65° C. In some embodiments, hybridization conditions comprise hybridization in a roller tube for at least 12 hours at 42° C. In each of the above conditions, the sodium phosphate hybridization buffer can be replaced by a hybridization buffer comprising 6×SSC (or 6×SSPE), 5×Denhardt's reagent, 0.5% SDS, and 100 g/ml carrier DNA, including 0-50% formamide, with hybridization and wash temperatures chosen based upon the desired stringency. Other hybridization and wash conditions are known to those of skill in the art (see also Sambrook & Russell, 2001; Ausubel et al., 2002; and Ausubel et al., 2003; each of which is incorporated herein in its entirety). As is known in the art, the addition of formamide in the hybridization solution reduces the Tₘ by about 0.4° C. Thus, high stringency conditions include the use of any of the above solutions and 0% formamide at 65° C., or any of the above solutions plus 50% formamide at 42° C.

For some high-density glass-based microarray experiments, hybridization at 65° C. is too stringent for typical use, at least in part because the presence of fluorescent labels destabilizes the nucleic acid duplexes (Randolph & Waggoner, 1995). Alternatively, hybridization can be performed in a formamide-based hybridization buffer as described in Piétu et al., 1996.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. Nos. 6,017,696 to Heller and 6,245,508 to Heller & Sosnowski.

II.A.5.c. Hybridization in Solution

In some embodiments of the presently disclosed subject matter, an amplified and/or labeled nucleic acid sample is hybridized to one or more probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5 M NaCl at 65° C. An example of stringent wash conditions is 15 minutes in 0.2×SSC buffer at 65° C. (see Sambrook and Russell, 2001, for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1M Na⁺ ion, typically about 0.01 M to 1 M Na⁺ ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single pathogen-specific probe set is hybridized to an amplified and labeled RNA sample derived from a target nucleic acid sample. Following hybridization, an antibody that recognizes DNA:RNA hybrids is used to precipitate the hybrids for subsequent analysis. The presence of the pathogen is determined by detection of the label in the precipitate.

Alternate capture techniques can be used as will be understood to one of skill in the art, for example, purification by a metal affinity column when using probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To assess the expression of multiple genes and/or samples from multiple different sources simultaneously, probes or probe sets can be distinguished by differential labeling of probes or probe sets. Alternatively, probes or probe sets can be spatially separated in different hybridization vessels.

In some embodiments, a probe or probe set having a unique label is prepared for each gene or source to be detected. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech of Piscataway, N.J., United States of America), which can be analyzed with good contrast and minimal signal leakage.

A unique label for each probe or probe set can further comprise a labeled microsphere to which a probe or probe set is attached. A representative system is LabMAP (Luminex Corporation of Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods of the presently disclosed subject matter, an individual pathogen-specific probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the randomly amplified, labeled nucleic acid sample with a set of microspheres comprising pathogen-specific probe sets, the hybridization mixture is analyzed to detect the signal of the color-code as well as the label of a sample nucleic acid bound to the microsphere. See Vignali 2000; Smith et al., 1998; and PCT International Patent Application Publication Nos. WO 01/13120; WO 01/14589; WO 99/19515; WO 99/32660; and WO 97/14028.

VII.A.6. Detection

Methods for detecting hybridization are typically selected according to the label employed.

In the case of a radioactive label (e.g., ³²P-dNTP) detection can be accomplished by autoradiography or by using a phosphorimager as is known to one of skill in the art. In some embodiments, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al., 1996.

In some embodiments, a nucleic acid sample or probe is labeled with far infrared, near infrared, or infrared fluorescent dyes. Following hybridization, the mixture of nucleic acids and probes is scanned photoelectrically with a laser diode and a sensor, wherein the laser scans with scanning light at a wavelength within the absorbance spectrum of the fluorescent label, and light is sensed at the emission wavelength of the label. See U.S. Pat. Nos. 6,086,737 to Patonav et al.; 5,571,388 to Patonav et al.; 5,346,603 to Middendorf & Brumbaugh; 5,534,125 to Middendorf et al.; 5,360,523 to Middendorf et al.; 5,230,781 to Middendorf & Patonav; 5,207,880 to Middendorf & Brumbaugh; and 4,729,947 to Middendorf & Brumbaugh. An ODYSSEY™ infrared imaging system (Li-Cor, Inc. of Lincoln, Nebr., United States of America) can be used for data collection and analysis.

If an epitope label has been used, a protein or compound that binds the epitope can be used to detect the epitope. For example, an enzyme-linked protein can be subsequently detected by development of a colorimetric or luminescent reaction product that is measurable using a spectrophotometer or luminometer, respectively.

In some embodiments, INVADER® technology (Third Wave Technologies of Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. Nos. 5,846,717 to Brow et al.; 5,985,557 to Prudent et al.; 5,994,069 to Hall et al.; 6,001,567 to Brow et al.; and 6,090,543 to Prudent et al.

In some embodiments, target nucleic acid/probe complexes are detected using an amplifying molecule, for example a poly-dA oligonucleotide as described by Lisle et al., 2001. Briefly, a tethered probe is employed against a target nucleic acid having a complementary nucleotide sequence. A target nucleic acid having a poly-dT sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

The presently disclosed subject matter also envisions use of electrochemical technology for detecting a nucleic acid hybrid according to the disclosed method. In this case, the detection method relies on the inherent properties of DNA, and thus a detectable label on the target sample or the probe/probe set is not required. In some embodiments, probe-coupled electrodes are multiplexed to simultaneously detect multiple genes using any suitable microarray or multiplexed liquid hybridization format. To enable detection, gene-specific and control probes are synthesized with substitution of the non-physiological nucleic acid base inosine for guanine, and subsequently coupled to an electrode. Following hybridization of a nucleic acid sample with probe-coupled electrodes, a soluble redox-active mediator (e.g., ruthenium 2,2′-bipyridine) is added, and a potential is applied to the sample. In the absence of guanine, each mediator is oxidized only once. However, when a guanine-containing nucleic acid is present, by virtue of hybridization of a sample nucleic acid molecule to the probe, a catalytic cycle is created that results in the oxidation of guanine and a measurable current enhancement. See U.S. Pat. Nos. 6,127,127 to Eckhardt et al.; 5,968,745 to Thorp et al.; and 5,871,918 to Thorp et al.

Surface plasmon resonance spectroscopy can also be used to detect hybridization. See e.g., Heaton et al., 2001; Nelson et al., 2001; and Guedon et al., 2000.

VII.B. Amino Acid-Based Assay Formats

The genes identified as being differentially expressed in ccA versus ccB type kidney cancer can also be used in a variety of peptide and/or polypeptide detection assays to detect or quantitate the expression level of a gene or multiple genes in a given sample. In some embodiments, methods and assays of the presently disclosed subject matter are employed with array or chip hybridization-based methods for detecting the expression of a plurality of genes.

Thus, an array for use in the presently disclosed subject matter can comprise peptides or polypeptides encoded by one or more of the genes listed in Table 7 instead of or in addition to polynucleotides. Briefly, a peptide and/or polypeptide array can be produced that includes peptides or polypeptides that comprise a subsequence of any or all of the polypeptides encoded by the genes listed in Table 7. Each such peptide or polypeptide can be placed in a different addressable location (i.e., “spot”) on the array, and different spots can include in some embodiments different peptides from the same gene product from Table 7 so that the array is internally redundant with respect to any or all gene products to be assayed. In some embodiments, the amount of peptide or polypeptide spotted on each location is reflective of the expression of the corresponding gene product in the cell or tissue to be assayed such that expression data from different assays can be compared. Methods for the production and use of peptide and polypeptide arrays that are appropriate for gene expression profiling are described, for example, in U.S. Patent Application Publication Nos. 20020009767; 20020155495; 20030049701; 20040033625; 20040219575; 20050255491; 20060275851; 20070099254; 20080260763; and 20090062194, each of which is incorporated by reference in its entirety.

VII.C. Data Analysis

Databases and software designed for use with use with microarrays is discussed in U.S. Pat. No. 6,229,911 to Balaban & Aggarwal, a computer-implemented method for managing information, stored as indexed tables, collected from small or large numbers of microarrays, and U.S. Pat. No. 6,185,561 to Balaban & Khurgin, a computer-based method with data mining capability for collecting gene expression level data, adding additional attributes and reformatting the data to produce answers to various queries. U.S. Pat. No. 5,974,164 to Chee, disclose a software-based method for identifying mutations in a nucleic acid sequence based on differences in probe fluorescence intensities between wild type and mutant sequences that hybridize to reference sequences.

Analysis of microarray data can also be performed using the method disclosed in Tusher et al., 2001, which describes the Significance Analysis of Microarrays (SAM) method for determining significant differences in gene expression among two or more samples.

### VIII. COMPOSITIONS FOR USE IN THE PRESENTLY DISCLOSED METHODS

The presently disclosed subject matter also provides compositions that can be employed in the practice of the methods disclosed herein.

The methods disclosed herein relate in some embodiments to generating gene expression profiles from biological samples that comprise kidney cancer cells obtained from a subject. The gene expression profiles are then in some embodiments compared to standards such as, but not limited to gene expression profiles of ccA cancer cells and/or ccB cancer cells. This comparison permits a physician to more accurately predict the degree to which a given subject is likely to benefit from particular treatment of the cancer, which info can then assist the subject in making informed decisions as to the course of his or her treatment.

As such, the presently disclosed methods can employ various techniques to generate the gene expression profiles required for the comparisons. See e.g., PCT International Patent Application Publication Nos. WO 2004/046098; WO 2004/110244; WO 2006/089268; WO 2007/001324; WO 2007/056332; WO 2007/070252, each of which is incorporated herein by reference in its entirety.

Generally, a gene expression profile can be generated using the following basic steps:
(1) a biological sample such as, but not limited to a kidney cancer biopsy or resected cancer cells are obtained; and
(2) the expression levels of three or more of the genes set forth in Table 7 (such as, but not limited to FLT1, FZD1, GIPC2, MAP7, and/or NPR3 genes) are determined.

As is known to one of ordinary skill in the art, gene expression levels can be assayed at the level of RNA and/or at the level of protein. As such, in some embodiments RNA is extracted from the biological sample and analyzed by techniques that include, but are not limited to PCR analysis (in some embodiments, quantitative reverse transcription PCR) and/or array analysis. In each case, one of ordinary skill in the art would be aware of techniques that can be employed to determine the expression level of a gene product in the biological sample.

With respect to PCR analyses, the sequences of nucleic acids that correspond to exemplary FLT1, FZD1, GIPC2, MAP7, and/or NPR3 gene products are present within the GENBANK® database (a subset of which are also provided in the Sequence Listing), and oligonucleotide primers can be designed for the purpose of determining expression levels.

Alternatively, arrays can be produced that include single-stranded nucleic acids that can hybridize to any or all of the gene products disclosed in Table 7 (e.g., FLT1, FZD1, GIPC2, MAP7, and/or NPR3 gene products). Exemplary, non-limiting methods that can be used to produce and screen arrays are described in Section VII hereinabove.

Therefore, in some embodiments the presently disclosed subject matter provides arrays comprising polynucleotides that are capable of hybridizing to at least five genes selected from among those disclosed in Table 7 including, but not limited to FLT1, FZD1, GIPC2, MAP7, and/or NPR3 or comprising specific peptide or polypeptide gene products of at least five of the genes disclosed in Table 7 (e.g., FLT1, FZD1, GIPC2, MAP7, and/or NPR3).

Alternatively or in addition, gene expression can be assayed by determining the levels at which polypeptides are present in kidney cancer tissue. This can also be done using arrays, and exemplary methods for producing peptide and/or polypeptide arrays in attached to nitrocellulose-coated glass slides (Espejo et al., 2002), alkanethiol-coated gold surfaces (Houseman et al., 2002), poly-L-lysine-treated glass slides (Haab et al., 2001), aldehyde-treated glass slides (MacBeath & Schreiber, 2000; Salisbury et al., 2002), silane-modified glass slides (Fang et al., 2002; Seong, 2002), and nickel-treated glass slides (Zhu et al., 2001), among others, have been reported.

In some embodiments the presently disclosed subject matter provides arrays that comprise peptides or polypeptides that are correspond to gene products from three or more of the genes listed in Table 7 (e.g., FLT1, FZD1, GIPC2, MAP7, and/or NPR3). In these embodiments, arrays are produced from proteins isolated from kidney cancer tissue, and these arrays are then probed with molecules that specifically bind to the various gene products of interest, if present. Exemplary molecules that specifically bind to FLT1, FZD1, GIPC2, MAP7, and/or NPR3 gene products include antibodies (as well as fragments and derivatives thereof that include at least one Fab fragment). Antibodies to human one or more of the polypeptides encoded by the genes listed in Table 7 are commercially available, and antibodies that specifically bind to these and other gene products can be produced using routine techniques.

Peptide and/or polypeptide arrays can be designed quantitatively such that the amount of each individual peptide or polypeptide is reflective of the amount of that individual peptide or polypeptide in the kidney cancer tissue.

Further, the arrays can be designed such that specific peptide or polypeptide gene products that correspond to three or more of the polypeptides encoded by the genes listed in Table 7 (e.g., FLT1, FZD1, GIPC2, MAP7, and/or

NPR3) can be localized (sometimes referred to as “spotted”) on the array such that the array is interrogatable with at least one antibody that specifically binds to one of the specific peptide or polypeptide gene products. In some embodiments, gene expression at the level of protein is assayed without isolating the relevant peptides and/or polypeptides from the kidney cancer cells. For example, immunohistochemistry and/or immunocytochemistry can be employed, in which the expression levels of gene products that correspond to three or more of the genes listed in Table 7 (e.g., FLT1, FZD1, GIPC2, MAP7, and/or NPR3) can be determined by incubating appropriate binding molecules to kidney cancer cells and/or tissue. In some embodiments, the kidney cancer cells and/or tissue are mounted in paraffin blocks before the immunohistochemistry and/or immunocytochemistry is performed.

### EXAMPLES

The following Examples provide further illustrative embodiments. In light of the present disclosure and the general level of skill in the art, those of skill will appreciate that the following Example is intended to be exemplary only and that numerous changes, modifications, and alterations can be employed without departing from the scope of the presently disclosed subject matter.

### Materials and Methods Employed in the Examples

Samples.

51 specimens from 48 ccRCC patients were collected from consenting patients undergoing nephrectomy for RCC from 1994-2008 (see Table 5 below), analyzed for quality, flash frozen, and accessed with appropriate IRB approvals. The validation set of 177 cases was described previously (Zhao et al., 2006). Survival data were updated with median follow-up of 120 months (range 66 to 271). The pVHL and HIF annotated dataset was previously described (Gordan et al., 2008).

Gene Expression Analysis.

RNA was extracted from fresh frozen tumor specimens (with independent replicates—separate sample preparations—of 3 tumors) and 18 specimens from adjacent normal kidney using the Qiagen RNeasy kit (Valencia, Calif.). The concentration of the purified RNA was measured on a Nanodrop ND-1000 (Thermo Scientific, Wilmington, Del.), and quality was assured using an Agilent 2100 Bioanalyzer (Agilent Technologies, Palo Alto, Calif.). The RNA samples were processed for amplification, label integration, and hybridization against a modified commercial reference RNA (Perou et al., 2000) on Agilent Whole Human Genome (4×44k) Oligo Microarrays (Aglient Technologies, Inc., Santa Clara, Calif., United States of America; the contents of these micrarrays, available from). Microarrays were scanned using the Agilent Scanner model C. Fluorescence ratios were determined by Agilent feature extraction software. Expression data were tabulated, and missing data were imputed. Batches were combined using Distance Weighted Discrimination (DWD; see the Community Participation section of the website for caBIG®, the CANCER BIOMEDICAL INFORMATICS GRID®, maintained by the National Cancer Institute of the National Institutes of Health of the United States of America) and normalized. Data are posted on GEO (GSE16449). Gene expression data from the validation set were collected (Zhao et al., 2006), GEO (GSE3538). Print runs DWD-combined and normalized. Gene expression data from the pVHL/HIF dataset23 were posted on GEO (GSE11904).

Data Normalization.

Expression data from the Agilent Arrays were tabulated in log₂ R/G Lowess normalized ratio (median) format, removing probes which had ≦70% good data (excluded if spot was not found in either channel, spot or spot background was a non-uniform outlier, spot or spot background was a non-uniform outlier for the population, spot was not a positive and significant signal in either channel, or Ch1 and 2 lowess normalized net (median)<10). Missing data was imputed using k-nearest neighbors method (k=10) using Significance Analysis of Microarrays (SAM; available from the website of Stanford University, Palo Alto, Calif., United States of America by searching “Significance Analysis of Microarrays”). The data for three groups of arrays, which were prepared in separate sample batches, was combined using Distance Weighted Discrimination (DWD; see the Community Participation section of the website for caBIG®).

Group 1: A4, A5, A6, A9, A10, A11, A13, A16, A18, A26, A26a, A27

Group 2: 2, 5, D3, D4, D5, D6, D8, D9, D10, E5, D11, E4, E6, E7, n6, n21, nC5

Group 3: 1, 3, 4, 6, 8, 11, 12, 15, 17, 21, 25, 27, 30, A28, A30, A31, A5a, A7, C1, C11, C11a, C13, C3, C5, C7, C9, n25, n27, n3, nA11, nA13, nA16, nA18, nA27, nA30, nA31, nA4, nA5, nA9, nC1, nC13

DWD is a tool that performs statistical corrections to reduce systematic biases resulting from different sources of RNA, batches of microarrays etc. It is generally used when combing data from different microarray platforms, but is also valuable to correct for possible biases introduced due to batch handling effects in data generated on the same platform in the same lab. These data are posted on GEO (GSE16449).

The 177 tumor validation set included gene expression data from ccRCC specimens from a previously published paper (Alexe et al., 2006), which is also available on GEO (GSE3538). It was tabulated and imputed as described above. This data included 10 print runs, which were also combined by DWD as above. Arrays were then standard normalized by subtracting the mean of the array and dividing by the standard deviation.

The pVHL and HIF annotated dataset was composed of 21 ccRCC specimens previously described (Gordan et al., 2008) and available on GEO (GSE11904). Arrays were normalized as above.

Pathway Analysis.

SAM was performed, and genes were selected using a cutoff of False Discovery Rate (FDR)<0.000001. Heat maps were generated using Cluster 3.0 (available through the World Wide Web by searching “Cluster 3.0”; de Hoon et al., 2004) and Java TreeView (available through the World Wide Web by searching “Java TreeView”). Differentially regulated genes were functionally annotated in DAVID Bioinformatics Database (Huang et al., 2009) with p value and FDR<0.05. SAM-GSA was also performed on the data using the curated gene sets from MSigDB (available from the Broad Institute of the Massachusetts Institute of Technology through the World Wide Web by searching “MSigDB”).

Feature Set Reduction by Principal Component Analysis (PCA).

PCA (Skubitz et al., 2006; Nogueira & Kim, 2008) is a feature selection method which reduces the feature set to those which have significant variation within the sample set. It is essentially a coordinate transformation in feature space which identifies a sorted list of “Principal Components”, which are linear combinations of the original features. The starting point of the analysis was the expression matrix Eᵢⱼ where the rows were samples and columns were genes. The analysis proceeded by computing the eigenvalues and eigenvectors of the correlation matrix between feature pairs across samples after Eᵢⱼ was centered and scaled to mean 0 and variance 1 per column. The higher the eigenvalue of the correlation matrix, the greater the variation represented by the direction in feature space defined by its eigenvector. The eigenvalues λᵢ were sorted in decreasing order and the k largest eigenvalues representing a fraction p of the variation in the data were identified by solving [Σᵢ₌₁^{k}λᵢ]=ρ[Σᵢ₌₁^{N}λᵢ] where N is the total number of genes. ρ=0.85 was selected; the results were not sensitive to this choice. From an examination of the coefficients of the genes in the eigenvectors for these eigenvalues, the subset of useful genes was identified as those with coefficients in the top 25% in absolute value in these k eigenvectors. In the 48 tumors plus three replicates dataset, this identified 26 eigenvectors and 347 features which were retained for further analysis.

Unsupervised Consensus Ensemble Clustering.

Unsupervised clustering algorithms divide data into groups such that the intra-cluster similarity is maximized and the inter-cluster similarity is minimized. For gene expression data, unsupervised clustering can be performed for genes, for arrays, or for both. Several types of clustering techniques are available to group data into sets. These can be divided into hierarchical, partitioning, probabilistic and grid-based methods. Consensus ensemble clustering (Sorlie et al., 2001) is a relatively recent method which uses a weighted combination of these methods to improve the quality and the robustness of the clusters identified by each individual technique. The consensus ensemble approach involved two methods: first, a method that generated a collection of clustering solutions, and second, a method that robustly combined the solutions to produce a single “best” clustering solution for the data. Unlike standard clustering techniques for which solutions divide all the data samples into groups, ensemble consensus clustering identified “core” groups of samples within clusters. These were samples which were consistently clustered into the same group, independent of perturbations of the data and of the choice of clustering methods used. This facilitated the identification of strong signatures of gene expression within each core cluster which could then be used to classify the remaining samples. It also provided a robust (perturbation independent) characterization of the gene expressions which distinguished the disease classes identified. Often a study of these genes which have noise independent differential expression between disease classes allows a better understanding of the underlying biological mechanisms driving the subtypes.

Several techniques were employed to create robust “core” clusters. If the clustering method was stochastic, the effect of stochastic variation was reduced by applying the clustering method repeatedly and taking an appropriate average. To reduce the sensitivity of the results to random variation in the data, each clustering method was applied to multiple sample datasets obtained by bootstrapping both the features (genes/probes) as well as the samples clustered. The core clusters were identified as those groups for which memberships consisted of samples consistently classified into the same group over all the bootstrap and clustering experiments. A new software suite called ConsensusCluster, which implements PCA and consensus ensemble clustering, was produced for this purpose and is available on the World Wide Web (search “ConsensusCluster”).

Consensus ensemble clustering was applied to data limited to the 347 features identified by PCA and the data was split into k=2, 3, 4 . . . clusters, which were made insensitive to data and clustering method bias by bootstrapping over many datasets and averaging over two clustering techniques: K-Means (Furge et al., 2004) and Self-Organizing Map (SOM; Takahashi et al., 2001).

The detailed procedure used was as follows:

Step 1. 75 datasets were created from the imputed data restricted to the 347 significant features identified by PCA. 75 datasets came from bootstrapping the samples, 75 from bootstrapping genes and 75 by first projecting the data on bootstrapped genes and then by further bootstrapping on samples.

Step 2. k=2,3,4 clusters were created for each dataset using k-means and SOM.

Step 3. For each k and each method, the k resulting clusters were combined into an agreement matrix Aᵢⱼ of size n×n.

Step 4. For each k, the samples were clustered using dᵢⱼ=1-Aᵢⱼ as a distance measure using hierarchical clustering and the hierarchical tree was truncated at the k^{th} level.

Logical Analysis of Data (LAD).

Logical analysis of data (Gordan et al., 2008; Reddy et al., 2008), is a method to find patterns distinguishing two classes. For gene expression data, LAD identifies patterns of expression which can stratify labeled data. It has been successfully used in several biomedical studies (Jolliffe, 2002; Monti et al., 2003; Paik et al., 2004).

As employed with respect to the presently disclosed subject matter, a pattern was a rule based on cutpoints in the expression of genes which could distinguish two subtypes ccA and ccB. A pattern was characterized by its degree, prevalence, and homogeneity. The degree was defined as the number of genes appearing in its defining conditions. The prevalence of a pattern was defined as the percent of positive (negative) cases which satisfy the pattern. The homogeneity of a pattern was defined as the percentage of positive (negative) cases covered by it. In general, patterns useful for classification had low degree and high prevalence and homogeneity.

To develop patterns to distinguish ccA and ccB, the complete set of probes on the Agilent chip was employed so as not to bias the analysis in any way. Each sample array was first standard normalized by subtracting the mean of the array and dividing by the standard deviation, in order to create patterns applicable to other datasets. Only those features that could discriminate the subtypes using a t-test at p-value <0.000001 were retained, and only the probes which were mapped to known genes were kept. This reduced the dataset to 1075 probes, which included the set of 347 identified by PCA. LAD was applied using the implementation that is available at the website of Pierre Lemaire (Assistant Professor at Grenoble INP, School of Industrial Engineering). LAD patterns requiring only one gene for perfect discrimination were generated in Leave-One-Out experiments (LOO; discussed below) to further reduce the gene set to 120. These probes were re-normalized by median centering, and LAD was reapplied to identify patterns of degree 1 and degree 2 (homogeneity and prevalence=0.9) using a single cut-point at expression value 0.

These patterns were used to predict the samples initially set aside as non-core samples. A classifier C_{S}=f_{P}−f_{N} assigns an unknown sample S to a class, where f_{N}/f_{P} are the fraction of negative/positive patterns satisfied by S. If the LAD score (C_{S}) is negative/positive, the sample is predicted to class ccA/ccB respectively. Confidence levels were computed by running 100 bootstraps of 80% of the patterns from the entire set, and the LAD score was computed for each bootstrapped sample. The final LAD score was the average of 100 runs, and the confidence level was the percent of times the sample was predicted to be in ccA or ccB. Samples with confidence levels <0.75 were left as unclassified.

Leave-One-Out Analysis (LOO).

LOO is a procedure to test the accuracy of a classifier that distinguishes two labeled classes. One sample was left out, then the classifier was created from the remaining samples and used to predict the class of the sample left out. The procedure was then repeated for all possible selections of “left-out” samples. The prediction accuracy of the classifier was the average fraction of correct classifications across all choices of the “left-out” sample.

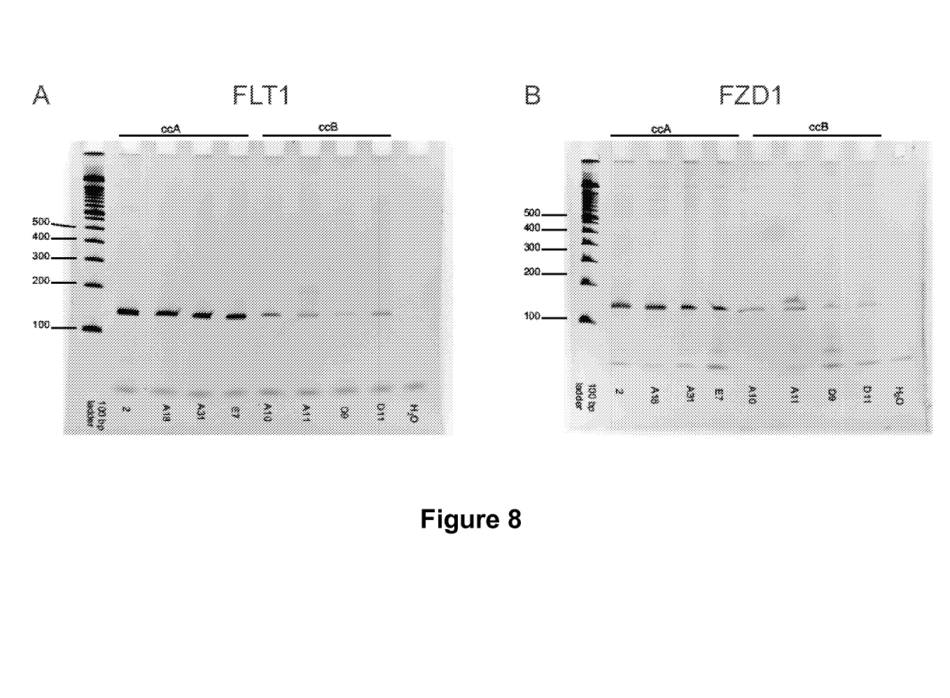

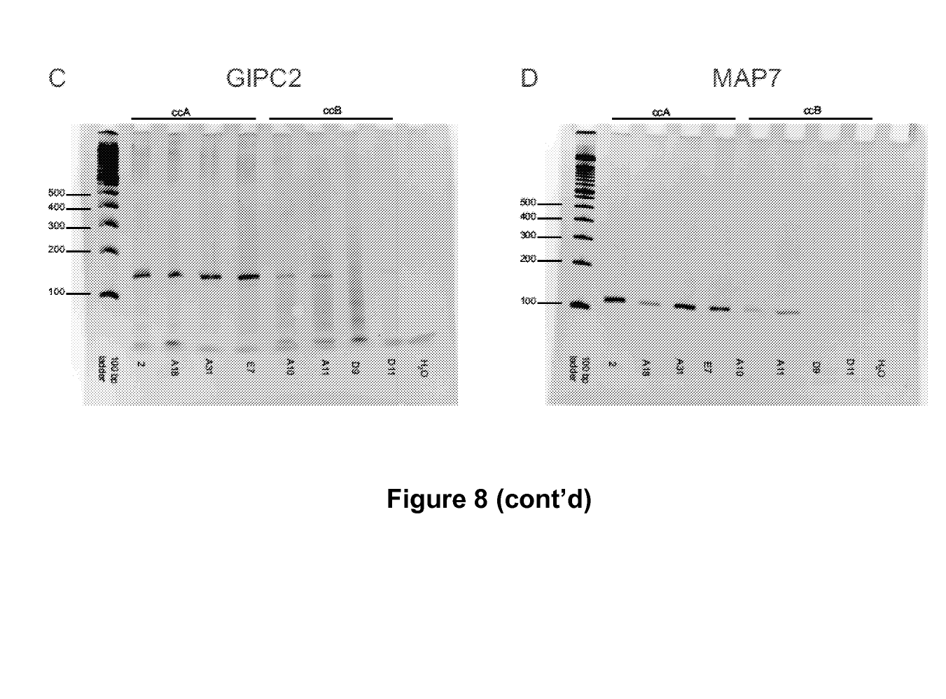

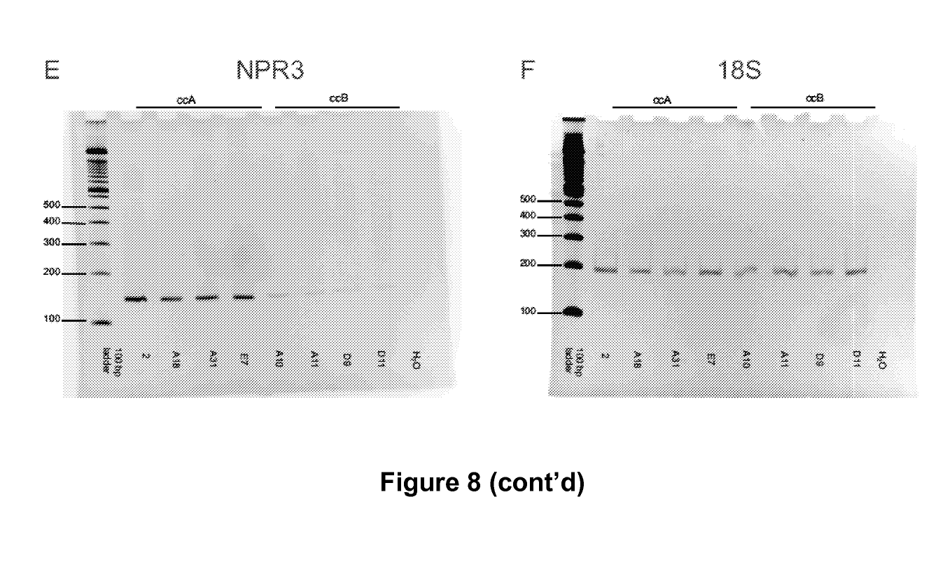

Semi-Quantitative Reverse Transcription PCR.

Where available, RNA was extracted from a second tumor sample from the same patient. Tumors were chosen based on RNA or tumor availability of RNA or tumor with the end goal of equal numbers in each subtype. 500 ng of total RNA from training set patient tumor samples was reverse transcribed using Superscript II polymerase (Invitrogen, Carlsbad, Calif.) using manufacturer recommended standard buffer and temperature conditions. In a representative embodiment, a 1:5 cDNA dilution was amplified by 25 cycles of semi-quantitative PCR with primer sets for FLT1 (ACTTTTACCGAATGCCACC (SEQ ID NO: 11) and TGGTTACTCTCAAGTCAATCTTG (SEQ ID NO: 12)), FZD1 (CCATCAAGACCATCACCATC (SEQ ID NO: 13) and GCCGATAAACAGGTACACGA (SEQ ID NO: 14)), GIPC2 (CCTGAGATCAAAAGGTCCTG (SEQ ID NO: 15) and CTTCAAACATTGTGGTGGC; SEQ ID NO: 16)), MAP7 (GCTACAGATAAGAAAACCAGTGA (SEQ ID NO: 17) and GCTTTCCATTTCCCGGA (SEQ ID NO: 18)), and NPR3 (TCGGCAGTGACAGGAATT (SEQ ID NO: 19) and CCCGATGTTTTCCAAGGT (SEQ ID NO: 20)). Primers were designed using IDT (see the website for Integrated DNA Technologies, Inc., Coralville, Iowa, United States of America). 18S rRNA primers (Applied Biosystems) were used as a control. Each primer set was tested on an equal number of ccA and ccB samples. Equivalent quantities of the semi-quantitative RT-PCR samples were run on a 6% acrylamide gel. Full sized gels are shown in FIGS. 8A-8F.

VHL Sequence and Methylation Analysis.

DNA was extracted from tumor samples using proteinase K (Roche) and standard phenol/chloroform extraction. VHL exons were PCR-amplified and directly sequenced for mutations with a BigDye Terminator Cycle kit on a 3130 xl sequencer (Applied Biosystems). Primers and protocols used were described previously (Stolle et al., 1998). A CpG Wiz kit (Chemicon) and/or NotI digestion was used for methylation studies (Herman et al., 1994).

Statistical Methods.

All statistical analyses were performed using R v2.4.1 (http://www.r-project.org), SAS (SAS Institute, Inc, Cary, N.C.), and STATA (Statacorp, College Station, Tex.). The Kaplan-Meier (or product limit) method was used to estimate the time to event functions of disease specific survival and overall survival. Disease specific survival was defined as the time from the nephrectomy to death due to disease. Overall survival was defined as the time from nephrectomy to death from all causes. The log-rank test was used to test for differences between disease-specific and overall survival Kaplan-Meier curves. Univariable logistic regression was used to evaluate the relative strength of association of covariates, one at a time, on the outcome probability of being subtype ccA versus ccB. The covariates of interest here were performance status, tumor stage, and grade. Univariable and multivariable Cox regression was used to evaluate the strength of association of individual and multiple covariates on disease specific and overall survival. The covariates of interest in these models were performance status, tumor stage, Fuhrman grade, subtype (ccA/ccB, or ccA/ccB/unclassified), and LAD scores. Model fit was assessed using an approximation to Bayes factors known as the Schwartz Bayesian Criterion (SBC; Kass & Raftery, 1995).

### Example 1

### Identification of ccRCC Subtypes

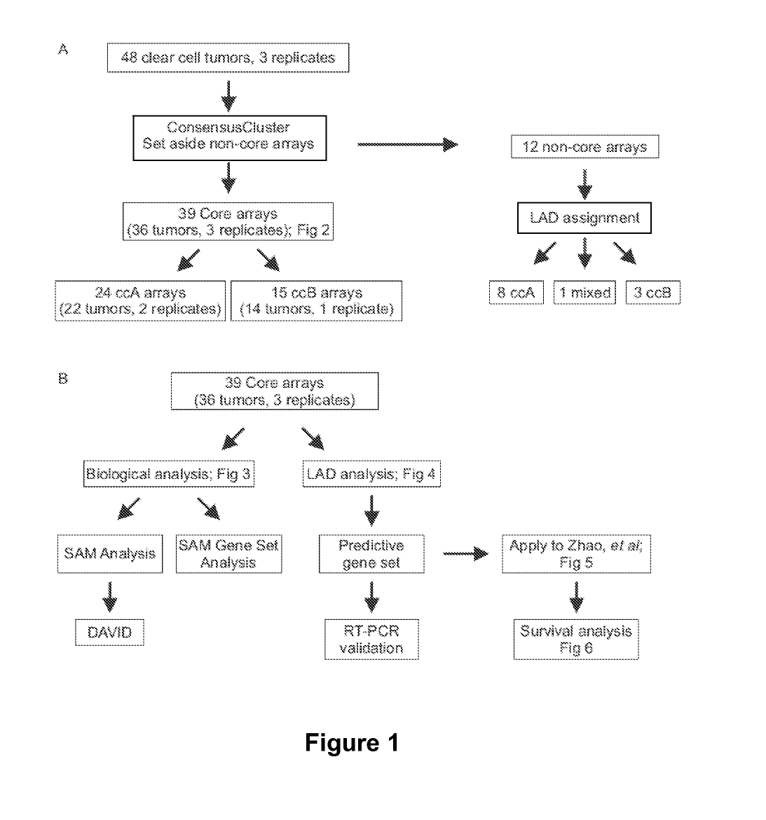

Gene expression data were obtained for 48 ccRCC samples and three independent replicate sample preparations. A flow-diagram depicting the analyses performed is presented in FIG. 1.

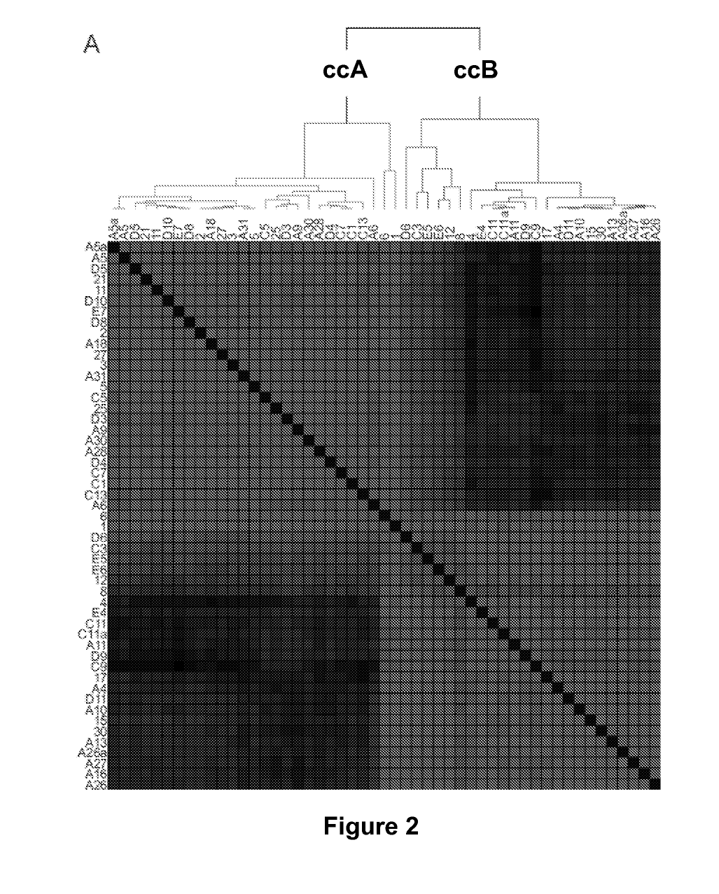

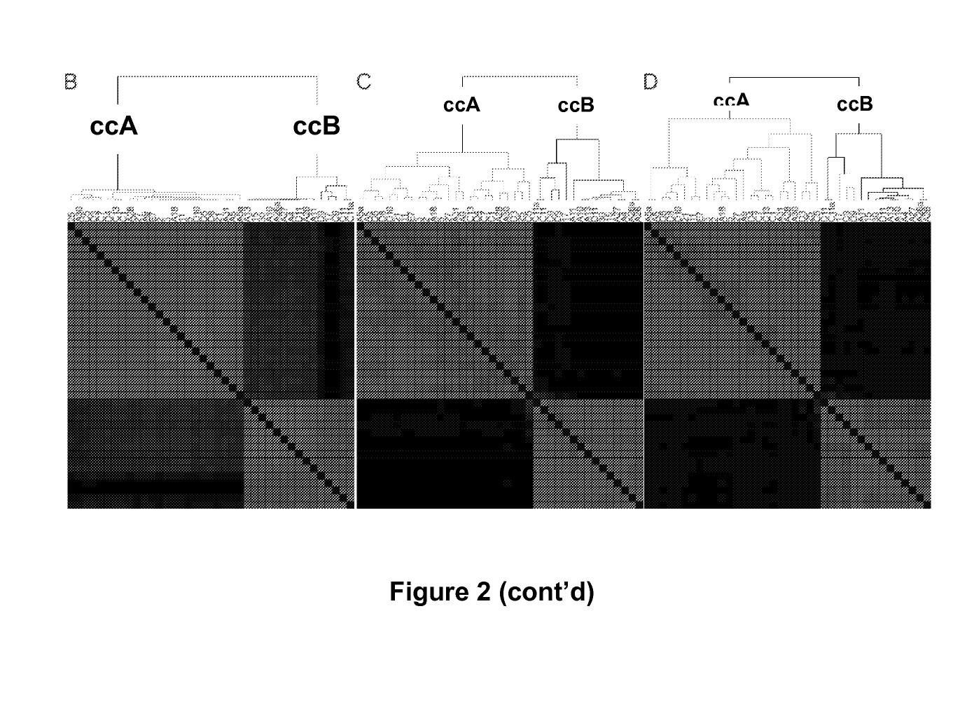

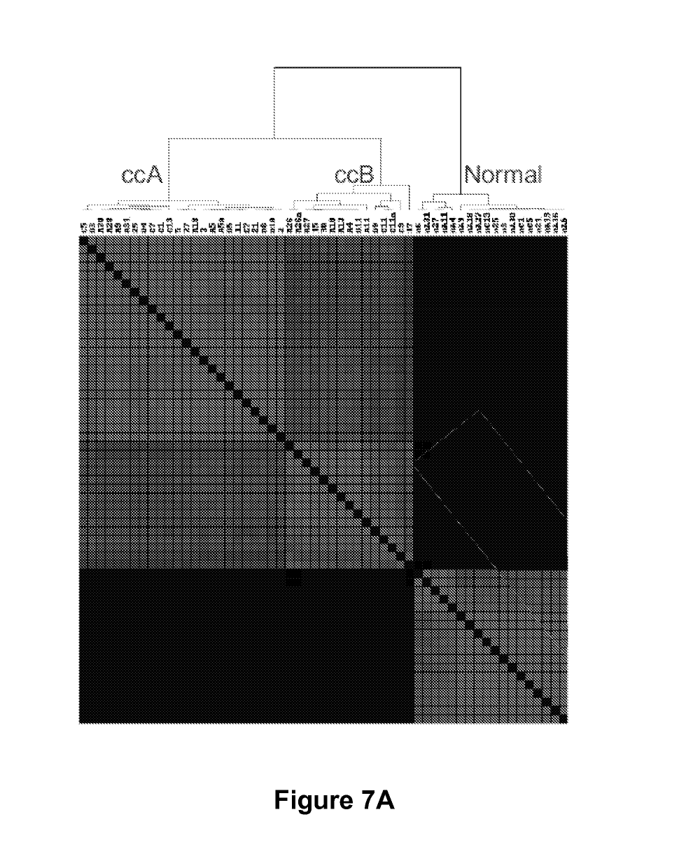

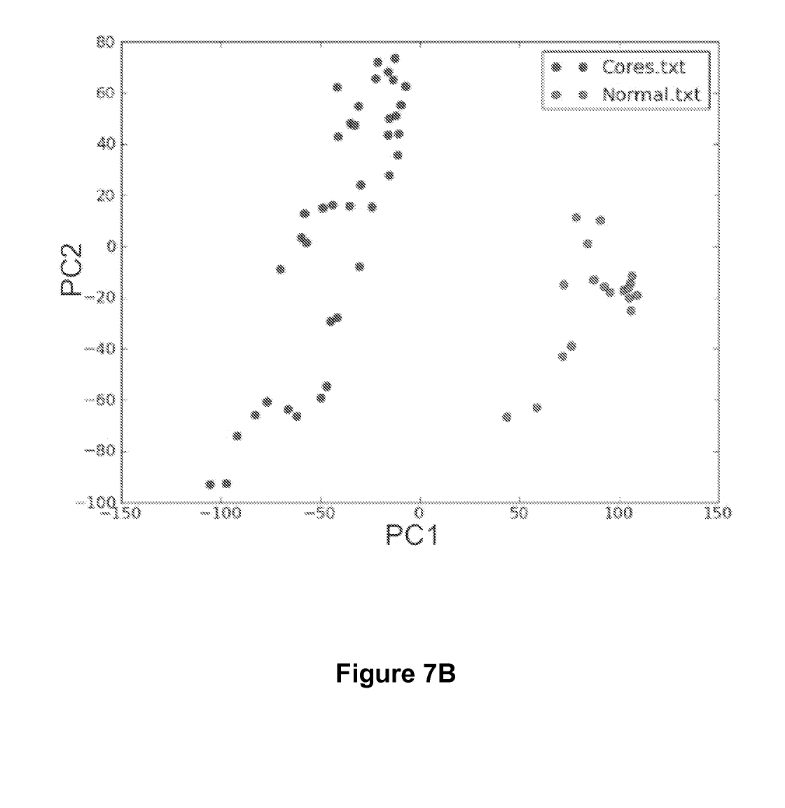

First, ConsensusCluster, an unsupervised ensemble clustering algorithm, was performed on 48 ccRCC samples and three independent replicate samples (see Table 1), yielding two subsets, designated ccA (n=24, with 22 tumors and 2 replicates) and ccB (n=15 with 14 tumors and 1 replicate; see FIG. 2A). Removing the independent replicates produced an identical clustering assignment of tumors, further confirming the stability of these clusters. Neither cluster was caused by inclusion of normal tissue in the RNA extraction as normal kidney assorts independently of either cluster (see FIGS. 7A and 7B).

TABLE 1
Tumor Characteristics for 51 Clear Cell Samples

| | | | | T- | VHL | VHL |
|---|---|---|---|---|---|---|
| Tumor | Core | Grade | Size | Stage | mutation | methylation |
| 2 | ccA | 2 | 5.2 | T1b | n/a | U |
| 3 | ccA | 2 | 2.5 | T1a | mutated | U |
| 5 | ccA | 2 | 6.1 | T1b | n/a | U |
| 11 | ccA | 2 | 4 | T1a | mutated | U |
| 21 | ccA | 2 | 4.4 | T1b | n/a | U |
| 25 | ccA | 2 | 4.7 | T1b | mutated | M |
| 27 | ccA | 2 | 4.5 | T1b | n/a | U |
| A18 | ccA | 2 | 7.5 | T2 | WT | n/a |
| A28 | ccA | 2 | 8 | T2 | nutated | U |
| A30 | ccA | 2 | 5.5 | T1b | WT | U |
| A31 | ccA | 2 | 2.7 | T1a | mutated | U |
| A5 | ccA | 3 | 17 | T3a | WT | U |
| A5a | ccA | 3 | 17 | T3a | WT | n/a |
| A9 | ccA | 2 | 8.2 | T3b | mutated | U |
| C1 | ccA | 3 | 2.2 | T1a | n/a | n/a |
| C13 | ccA | 3 | 4.7 | T1b | n/a | n/a |
| C5 | ccA | 2 | 2.7 | T1a | n/a | n/a |
| C7 | ccA | 3 | 2.8 | T1a | n/a | n/a |
| D10 | ccA | 2 | 3.5 | T1a | n/a | n/a |
| D3 | ccA | 2 | 5 | T1b | n/a | n/a |
| D4 | ccA | 1 | 5.5 | T1b | n/a | n/a |
| D5 | ccA | 2 | 4.1 | T1b | n/a | n/a |
| D8 | ccA | 2 | 3.8 | T1a | n/a | n/a |
| E7 | ccA | 2 | 5.5 | T1b | n/a | n/a |
| 15 | ccB | 2 | 5.5 | T1b | mutated | U |
| 17 | ccB | 2 | 3 | T1a | WT | U |
| 30 | ccB | 3 | 7 | T1b | WT | U |
| A10 | ccB | 2 | 3.2 | T1a | WT | U |
| A11 | ccB | 3 | 3 | T1a | WT | U |
| A13 | ccB | 3 | 10 | T3b | WT | U |
| A26 | ccB | 2 | 3 | T1a | WT | M |
| A26a | ccB | 2 | 3 | T1a | n/a | n/a |
| A27 | ccB | 2 | 2 | T1a | WT | n/a |
| A4 | ccB | 2 | 3.9 | T1a | n/a | U |
| C11 | ccB | 2 | 7.5 | T2 | n/a | n/a |
| C11a | ccB | 2 | 7.5 | T2 | n/a | n/a |
| C9 | ccB | 3 | 8.7 | T2 | n/a | n/a |
| D11 | ccB | 2 | 2.3 | T1a | n/a | n/a |
| D9 | ccB | 2 | 1.8 | T1a | n/a | n/a |
| 1 | (ccA) | 2 | 7.9 | T2 | WT | U |
| 6 | (ccA) | 2 | 4.3 | T1b | mutated | U |
| 12 | (ccA) | 3 | 8 | T2 | mutated | U |
| A6 | (ccA) | 2 | 3.8 | T1a | WT | M |
| C3 | (ccA) | 2 | 4.5 | T1b | n/a | n/a |
| D6 | (ccA) | 3 | 4.2 | T1b | n/a | n/a |
| E5 | (ccA) | 2 | 8 | T2 | n/a | n/a |
| E6 | (ccA) | 3 | 10.2 | T2 | n/a | n/a |
| 4 | (ccB) | 3 | 5 | T3b | n/a | U |
| A16 | (ccB) | 1 | 2.5 | T1a | WT | n/a |
| E4 | (ccB) | 2 | 3.5 | T1a | n/a | n/a |
| 8 | (unclass) | 3 | 4.5 | T3a | mutated | M |

Tumors suffixed with “a” were independent replicates. Arrays labeled in parentheses were assigned by pattern analysis using the 120 LAD probes. If labeled (unclass), the tumor could not be assigned using LAD pattern analysis.
Grade—Fuhrman nuclear grade (1-4).
Size—Tumor size (cm).
T-stage—Tumor stage according to pathology report.
WT—no nutations detected.
U—unmethylated.
M—methylated.
n/a—not available.

Representative samples within each cluster were used for the development of characteristic gene signatures and the decipherment of biological pathways. Samples whose membership shifted through multiple bootstrapped iterations were set aside for later classification. These “core” clusters included 39 of the original 51 samples, and permitted tumors with best patterned features to define the cluster.

As FIG. 2B shows, the core cluster samples split into two robust subtypes of ccRCC that are stable when k (degrees of freedom) increases to k=3 or k=4 (FIGS. 2C and 2D), suggesting that the optimal number of robust clusters in this dataset is two. These analyses demonstrate that ccRCC can be optimally clustered into two distinct subtypes (ccA and ccB), defined purely by molecular characteristics of the tumors.

### Example 2

### Analysis of Pathway Differences Between Two Core Clusters

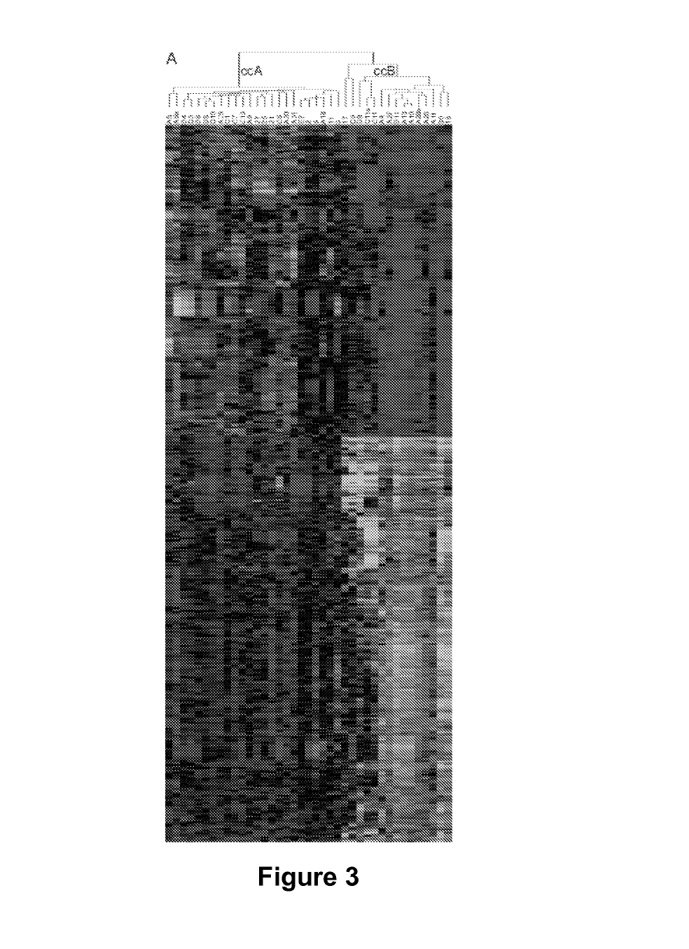

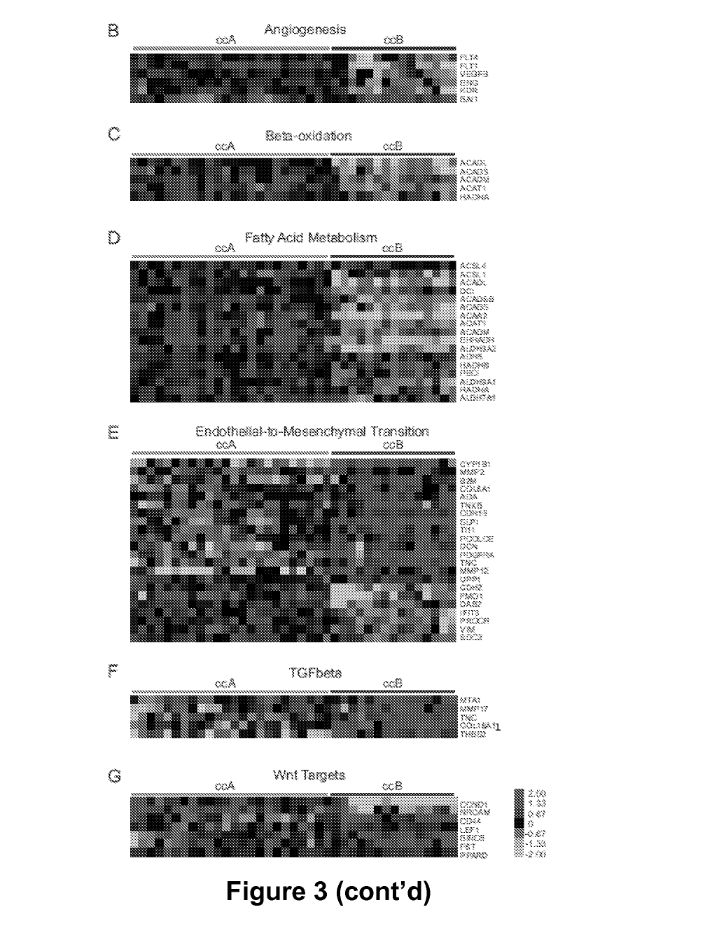

The identification of subtypes provides an opportunity to identify biological differences within the spectrum of ccRCC. SAM (Significance Analysis of Microarrays) analysis identified 2701 and 3512 probes over-expressed in ccA and ccB, respectively (see FIG. 3A and Tables 2 and 3). This result confirms the gene expression profile heterogeneity observed in previous studies (Takahashi et al., 2001; Skubitz et al., 2006; Zhao et al., 2006; Nogueira & Kim, 2008). The functional classification program, DAVID (available from the World Wide Web site of the United States National Institute of Allergy and Infectious Diseases (NIAID) of the Natuional Istitutes of Health (NIH)), was used to functionally categorize the probes identified in the presently disclosed analysis. A demonstration of the gene ontologies and pathways found to be differentially regulated between ccA and ccB tumors is provided in Tables 2 and 3. In Tables 2 and 3, individual pathways, processes, cellular components, molecular functions, etc. are listed by the identifiers provided by the Gene Ontology (GO) Project (i.e., the numbers that begin “GO:”). The GO Project maintains a searchable website on the World Wide Web that includes a listing of all genes (e.g., all human genes) that are associated with the listed identifiers.

Additionally, SAM Gene Set Analysis, a more statistically robust way of identifying correlated gene groups, was performed using the Molecular Signatures Database (MSigDB) curated gene sets, providing similar results (see Tables 4 and 5). The most notable genes, gene sets, and gene ontologies associated with cluster ccA were involved in angiogenesis (FIG. 3B), the beta-oxidation pathway (FIG. 3C), organic acid metabolism, fatty acid metabolism (FIG. 3D), and pyruvate metabolism. In contrast, core cluster ccB tumors overexpressed genes associated with cell differentiation, epithelial to mesenchymal transition (EMT; FIG. 3E), the mitotic cell cycle, TGFβ (FIG. 3F), response to wounding, and Wnt targets (FIG. 3G).

TABLE 2
Pathways Overexpressed in ccA Tumors

| | | Fold | | | |
|---|---|---|---|---|---|
| Term | p Value | Enrichment | Bonferroni | Benjamini | FDR |
| GO:0008152~metabolic | 3.9 × 10⁻¹² | 1.13 | 2.1 × 10⁻⁸ | 2.1 × 10⁻⁸ | 7.5 × 10⁻¹¹ |
| process | | | | | |
| GO:0019752~carboxylic acid | 4.0 × 10⁻⁸ | 1.78 | 2.1 × 10⁻⁵ | 1.1 × 10⁻⁵ | 7.7 × 10⁻⁸ |
| metabolic process | | | | | |
| GO:0006082~organic acid | 5.6 × 10⁻⁸ | 1.78 | 2.9 × 10⁻⁵ | 9.7 × 10⁻⁶ | 1.1 × 10⁻⁷ |
| metabolic process | | | | | |
| GO:0009058~biosynthetic | 1.6 × 10⁻⁸ | 1.42 | 8.2 × 10⁻⁵ | 2.1 × 10⁻⁵ | 3.0 × 10⁻⁷ |
| process | | | | | |
| GO:0006629~lipid metabolic | 4.9 × 10⁻⁸ | 1.60 | 0.00026 | 5.2 × 10⁻⁵ | 9.4 × 10⁻⁷ |
| process | | | | | |
| GO:0044237~cellular | 1.0 × 10⁻⁷ | 1.11 | 0.00055 | 9.1 × 10⁻⁵ | 2.0 × 10⁻ [symbol] |
| metabolic process | | | | | |
| GO:0044255~cellular lipid | 1.2 × 10⁻⁷ | 1.66 | 0.00063 | 9.0 × 10⁻⁵ | 2.3 × 10⁻ [symbol] |
| metabolic process | | | | | |
| GO:0033036~macromolecule | 2.5 × 10⁻⁷ | 1.54 | 0.0013 | 0.00016 | 4.8 × 10⁻⁶ |
| localization | | | | | |
| GO:0015031~protein | 3.2 × 10⁻⁷ | 1.60 | 0.0017 | 0.00019 | 6.2 × 10⁻⁶ |
| transport | | | | | |
| GO:0008104~protein | 3.4 × 10⁻⁷ | 1.55 | 0.0018 | 0.00018 | 6.5 × 10⁻⁶ |
| localization | | | | | |
| GO:0045184~establishment | 3.7 × 10⁻⁷ | 1.57 | 0.0019 | 0.00018 | 7.1 × 10⁻⁶ |
| of protein localization | | | | | |
| GO:0044238~primary | 4.6 × 10⁻⁷ | 1.11 | 0.0024 | 0.00020 | 8.8 × 10⁻⁶ |
| metabolic process | | | | | |
| GO:0044249~cellular | 9.2 × 10⁻⁷ | 1.43 | 0.0048 | 0.00037 | 1.8 × 10⁻⁵ |
| biosynthetic process | | | | | |
| GO:0046907~intracellular | 1.1 × 10⁻⁶ | 1.56 | 0.0059 | 0.00042 | 2.2 × 10⁻⁵ |
| transport | | | | | |
| GO:0019538~protein | 1.6 × 10⁻⁶ | 1.20 | 0.0082 | 0.00055 | 3.0 × 10⁻⁵ |
| metabolic process | | | | | |
| hsa00280: Valine, leucine and | 1.1 × 10⁻⁶ | 3.22 | 0.0023 | 0.0023 | 0.00014 |
| isoleucine degradation | | | | | |
| GO:0006631~fatty acid | 8.5 × 10⁻⁶ | 2.14 | 0.044 | 0.0028 | 0.00016 |
| metabolic process | | | | | |
| GO:0032787~monocarboxylic | 1.1 × 10⁻⁵ | 1.93 | 0.055 | 0.0033 | 0.00020 |
| acid metabolic process | | | | | |
| GO:0044260~cellular | 1.2 × 10⁻⁵ | 1.19 | 0.063 | 0.0036 | 0.00024 |
| macromolecule metabolic | | | | | |
| process | | | | | |
| GO:0051641~cellular | 1.5 × 10⁻⁵ | 1.43 | 0.075 | 0.0041 | 0.00028 |
| localization | | | | | |
| GO:0044267~cellular protein | 3.1 × 10⁻⁵ | 1.18 | 0.15 | 0.0082 | 0.00060 |
| metabolic process | | | | | |
| GO:0009059~macromolecule | 3.3 × 10⁻⁵ | 1.41 | 0.16 | 0.0083 | 0.00064 |
| biosynthetic process | | | | | |
| GO:0051649~establishment | 3.4 × 10⁻⁵ | 1.41 | 0.17 | 0.0082 | 0.00066 |
| of cellular localization | | | | | |
| GO:0016043~cellular | 3.6 × 10⁻⁵ | 1.21 | 0.17 | 0.0082 | 0.00069 |
| component organization | | | | | |
| and biogenesis | | | | | |
| GO:0006412~translation | 7.7 × 10⁻⁵ | 1.48 | 0.33 | 0.017 | 0.0015 |
| GO:0051179~localization | 0.00010 | 1.18 | 0.41 | 0.021 | 0.0019 |
| GO:0006635~fatty acid beta- | 0.00013 | 4.57 | 0.49 | 0.026 | 0.0025 |
| oxidation | | | | | |
| hsa00071: Fatty acid | 0.00026 | 2.80 | 0.051 | 0.026 | 0.0033 |
| metabolism | | | | | |
| GO:0019395~fatty acid | 0.00018 | 3.71 | 0.61 | 0.034 | 0.0034 |
| oxidation | | | | | |
| GO:0051234~establishment | 0.00024 | 1.18 | 0.72 | 0.044 | 0.0046 |
| of localization | | | | | |
| hsa03010: Ribosome | 0.00051 | 2.04 | 0.098 | 0.034 | 0.0064 |
| GO:0006810~transport | 0.00034 | 1.18 | 0.83 | 0.060 | 0.0065 |
| GO:0007031~peroxisome | 0.00043 | 4.00 | 0.90 | 0.073 | 0.0082 |
| organization and | | | | | |
| biogenesis | | | | | |
| GO:0006886~intracellular | 0.00046 | 1.54 | 0.91 | 0.075 | 0.0087 |
| protein transport | | | | | |
| GO:0006732~coenzyme | 0.00057 | 1.80 | 0.95 | 0.090 | 0.011 |
| metabolic process | | | | | |
| hsa00460: Cyanoamino acid | 0.0013 | 5.90 | 0.23 | 0.063 | 0.016 |
| metabolism | | | | | |
| GO:0008610~lipid | 0.0010 | 1.62 | 0.996 | 0.15 | 0.020 |
| biosynthetic process | | | | | |
| GO:0008654~phospholipid | 0.0011 | 2.36 | 0.997 | 0.16 | 0.021 |
| biosynthetic process | | | | | |
| GO:0009308~amine | 0.0011 | 1.46 | 0.997 | 0.16 | 0.021 |
| metabolic process | | | | | |
| GO:0051186~cofactor | 0.0014 | 1.67 | 0.999 | 0.18 | 0.026 |
| metabolic process | | | | | |
| GO:0006519~amino acid and | 0.0015 | 1.50 | 0.9996 | 0.19 | 0.028 |
| derivative metabolic | | | | | |
| process | | | | | |
| hsa00640: Propanoate | 0.0022 | 2.77 | 0.37 | 0.087 | 0.028 |
| metabolism | | | | | |
| hsa00310: Lysine degradation | 0.0023 | 2.41 | 0.37 | 0.074 | 0.028 |
| GO:0006505~GPI anchor | 0.0016 | 3.42 | 0.9997 | 0.19 | 0.029 |
| metabolic process | | | | | |
| GO:0009066~aspartate | 0.0016 | 3.75 | 0.9998 | 0.19 | 0.030 |
| family amino acid | | | | | |
| metabolic process | | | | | |
| GO:0006512~ubiquitin cycle | 0.0017 | 1.41 | 0.9999 | 0.20 | 0.032 |
| GO:0007179~transforming | 0.0018 | 2.63 | 0.9999 | 0.20 | 0.033 |
| growth factor beta | | | | | |
| receptor signaling pathway | | | | | |
| GO:0006888~ER to Golgi | 0.0020 | 2.40 | 0.99997 | 0.22 | 0.038 |
| vesicle-mediated transport | | | | | |
| GO:0006807~nitrogen | 0.0023 | 1.41 | 0.99999 | 0.24 | 0.043 |
| compound metabolic | | | | | |
| process | | | | | |
| G0:0001558~regulation of | 0.0024 | 1.81 | 0.999997 | 0.25 | 0.045 |
| cell growth | | | | | |
| GO:0009056~catabolic | 0.0024 | 1.32 | 0.999997 | 0.25 | 0.045 |
| process | | | | | |
| GO:0007178~transmembrane | 0.0024 | 2.21 | 0.999997 | 0.24 | 0.045 |
| receptor protein | | | | | |
| serine/threonine kinase | | | | | |
| signaling pathway | | | | | |
| GO:0044248~cellular | 0.0024 | 1.37 | 0.999997 | 0.24 | 0.046 |
| catabolic process | | | | | |
| GO:0006790~sulfur metabolic | 0.0026 | 2.14 | 0.999999 | 0.24 | 0.048 |
| process | | | | | |

[symbol]

indicates data missing or illegible when filed

TABLE 3
Pathways Overexpressed in ccB Tumors

| | | Fold | | | |
|---|---|---|---|---|---|
| Term | p Value | Enrichment | Bonferroni | Benjamini | FDR |
| GO:0000278~mitotic cell | 7.8 × 10⁻¹⁷ | 2.86 | 5.83 × 10⁻¹³ | 5.83 × 10⁻¹³ | 2.11 × 10⁻¹⁵ |
| cycle | | | | | |
| GO:0022403~cell cycle | 1.9 × 10⁻¹⁵ | 2.66 | 9.92 × 10⁻¹² | 5.00 × 10⁻¹² | 3.61 × 10⁻¹⁴ |
| phase | | | | | |
| GO:0022402~cell cycle | 3.5 × 10⁻¹⁵ | 2.06 | 1.80 × 10⁻¹¹ | 6.03 × 10⁻¹² | 6.58 × 10⁻¹⁴ |
| process | | | | | |
| GO:0007067~mitosis | 1.2 × 10⁻¹⁴ | 3.10 | 6.01 × 10⁻¹¹ | 1.50 × 10⁻¹¹ | 2.19 × 10⁻¹³ |
| GO:0065007~biological | 1.57 × 10⁻¹⁴ | 1.29 | 8.22 × 10⁻¹¹ | 1.64 × 10⁻¹¹ | 2.99 × 10⁻¹³ |
| regulation | | | | | |
| GO:0000087~M phase of | 1.74 × 10⁻¹⁴ | 3.07 | 9.10 × 10⁻¹¹ | 1.52 × 10⁻¹¹ | 3.31 × 10⁻¹³ |
| mitotic cell cycle | | | | | |
| GO:0000279~M phase | 3.23 × 10⁻¹⁴ | 2.79 | 1.70 × 10⁻¹⁰ | 2.42 × 10⁻¹¹ | 6.18 × 10⁻¹³ |
| GO:0007049~cell cycle | 7.64 × 10⁻¹⁴ | 1.90 | 4.01 × 10⁻¹⁰ | 5.02 × 10⁻¹¹ | 1.46 × 10⁻¹² |
| GO:0050789~regulation of | 3.81 × 10⁻¹³ | 1.30 | 2.00 × 10⁻⁹ | 2.23 × 10⁻¹⁰ | 7.30 × 10⁻¹² |
| biological process | | | | | |
| GO:0032502~developmental | 9.98 × 10⁻¹³ | 1.38 | 5.25 × 10⁻⁹ | 5.25 × 10⁻¹⁰ | 1.91 × 10⁻¹¹ |
| process | | | | | |
| GO:0048518~positive | 9.92 × 10⁻¹¹ | 1.68 | 5.21 × 10⁻⁷ | 4.74 × 10⁻⁸ | 1.90 × 10⁻⁹ |
| regulation of biological | | | | | |
| process | | | | | |
| GO:0009888~tissue | 6.42 × 10⁻¹⁰ | 2.29 | 3.37 × 10⁻⁶ | 2.81 × 10⁻⁷ | 1.23 × 10⁻⁸ |
| development | | | | | |
| GO:0051301~cell division | 1.48 × 10⁻⁹ | 2.52 | 7.78 × 10⁻⁶ | 5.99 × 10⁻⁷ | 2.83 × 10⁻⁸ |
| GO:0048856~anatomical | 1.69 × 10⁻⁹ | 1.42 | 8.86 × 10⁻⁶ | 6.33 × 10⁻⁷ | 3.22 × 10⁻⁸ |
| structure develoment | | | | | |
| GO:0050794~regulation of | 2.52 × 10⁻⁹ | 1.26 | 1.32 × 10⁻⁵ | 8.33 × 10⁻⁷ | 4.82 × 10⁻⁸ |
| cellular process | | | | | |
| GO:0048731~system | 3.02 × 10⁻⁹ | 1.47 | 1.58 × 10⁻⁵ | 9.90 × 10⁻⁷ | 5.77 × 10⁻⁸ |
| development | | | | | |
| GO:0048522~positive | 3.07 × 10⁻⁹ | 1.66 | 1.61 × 10⁻⁵ | 9.49 × 10⁻⁷ | 5.87 × 10⁻⁸ |
| regulation of cellular | | | | | |
| process | | | | | |
| GO:0030154~cell | 3.52 × 10⁻⁹ | 1.45 | 1.85 × 10⁻⁵ | 1.03 × 10⁻⁶ | 6.73 × 10⁻⁸ |
| differentiation | | | | | |
| GO:0048869~cellular | 3.52 × 10⁻⁹ | 1.45 | 1.85 × 10⁻⁵ | 1.03 × 10⁻⁶ | 6.73 × 10⁻⁸ |
| developmental process | | | | | |
| GO:0048513~organ | 3.93 × 10⁻⁹ | 1.56 | 2.06 × 10⁻⁵ | 1.03 × 10⁻⁶ | 7.51 × 10⁻⁸ |
| development | | | | | |
| GO:0051276~chromosome | 8.29 × 10⁻⁹ | 2.09 | 4.36 × 10⁻⁵ | 2.08 × 10⁻⁶ | 1.59 × 10⁻⁷ |
| organization and | | | | | |
| biogenesis | | | | | |
| GO:0007275~multicellular | 8.84 × 10⁻⁹ | 1.38 | 4.65 × 10⁻⁵ | 2.11 × 10⁻⁶ | 1.69 × 10⁻⁷ |
| organismal development | | | | | |
| GO:0000074~regulation of | 2.49E × 10⁻⁸ | 1.89 | 1.31 × 10⁻⁴ | 5.68 × 10⁻⁶ | 4.76 × 10⁻⁷ |
| progression through cell | | | | | |
| cycle | | | | | |
| GO:0051726~regulation of | 3.21 × 10⁻⁸ | 1.88 | 1.69 × 10⁻⁴ | 7.03 × 10⁻⁶ | 6.14 × 10⁻⁷ |
| cell cycle | | | | | |
| GO:0007059~chromosome | 7.80 × 10⁻⁷ | 4.04 | 4.10 × 10⁻⁴ | 1.64 × 10⁻⁵ | 1.49 × 10⁻⁶ |
| segregation | | | | | |
| GO:0009987~cellular | 1.03 × 10⁻⁷ | 1.06 | 5.39 × 10⁻⁴ | 2.07 × 10⁻⁵ | 1.96 × 10⁻⁶ |
| process | | | | | |
| GO:0043283~biopolymer | 1.34 × 10⁻⁷ | 1.19 | 7.03 × 10⁻⁴ | 2.60 × 10⁻⁵ | 2.56 × 10⁻ |
| metabolic process | | | | | |
| GO:0007398~ectoderm | 1.92 × 10⁻⁷ | 2.67 | 0.0010 | 3.61 × 10⁻⁵ | 3.68 × 10⁻⁶ |
| development | | | | | |
| GO:0006996~organelle | 3.33 × 10⁻⁷ | 1.50 | 0.0017 | 6.03 × 10⁻⁵ | 6.37 × 10⁻ [symbol] |
| organization and | | | | | |
| biogenesis | | | | | |
| GO:0008544~epidermis | 3.36 × 10⁻⁷ | 2.70 | 0.0018 | 5.88 × 10⁻⁵ | 6.42 × 10⁻ [symbol] |
| development | | | | | |
| GO:0016043~cellular | 4.13 × 10⁻⁷ | 1.30 | 0.0022 | 7.00 × 10⁻⁵ | 7.90 × 10⁻ [symbol] |
| component organization | | | | | |
| and biogenesis | | | | | |
| GO:0008283~cell | 2.31 × 10⁻⁶ | 1.58 | 0.012 | 3.79 × 10⁻⁴ | 4.42 × 10⁻ [symbol] |
| proliferation | | | | | |
| hsa04110: Cell cycle | 3.53 × 10⁻⁶ | 2.62 | 7.09 × 10⁻ [symbol] | 7.09 × 10⁻⁴ | 4.42 × 10⁻ [symbol] |
| GO:0002526~acute | 7.31 × 10⁻⁶ | 3.23 | 0.038 | 0.0012 | 1.40 × 10⁻⁴ |
| inflammatory response | | | | | |
| GO:0009653~anatomical | 9.02 × 10⁻⁶ | 1.44 | 0.046 | 0.0014 | 1.73 × 10⁻⁴ |
| structure morphogenesis | | | | | |
| GO:0006357~regulation of | 9.80 × 10⁻⁵ | 1.73 | 0.050 | 0.0015 | 1.87 × 10⁻⁴ |
| transcription from RNA | | | | | |
| polymerase II promoter | | | | | |
| GO:0007088~regulation of | 1.01 × 10⁻⁵ | 3.29 | 0.052 | 0.0015 | 1.93 × 10⁻⁴ |
| mitosis | | | | | |
| GO:0032501~multicellular | 1.02 × 10⁻⁵ | 1.21 | 0.052 | 0.0014 | 1.95 × 10⁻⁴ |
| organismal process | | | | | |
| GO:0016265~death | 1.83 × 10⁻⁵ | 1.51 | 0.092 | 0.0025 | 3.50 × 10⁻⁴ |
| GO:0008219~cell death | 1.83 × 10⁻⁵ | 1.51 | 0.092 | 0.0025 | 3.50 × 10⁻⁴ |
| GO:0006366~transcription | 2.05 × 10⁻⁵ | 1.58 | 0.10 | 0.0027 | 3.91 × 10⁻⁴ |
| from RNA polymerase II | | | | | |
| promoter | | | | | |
| GO:0000070~mitotic sister | 2.06 × 10⁻⁵ | 4.69 | 0.10 | 0.0026 | 3.94 × 10⁻⁴ |
| chromatid segregation | | | | | |
| GO:0048468~cell | 2.09 × 10⁻⁵ | 1.40 | 0.10 | 0.0026 | 4.00 × 10⁻⁴ |
| development | | | | | |
| GO:0043067~regulation of | 2.12 × 10⁻⁵ | 1.65 | 0.11 | 0.0026 | 4.05 × 10⁻⁴ |
| programmed cell death | | | | | |
| GO:0019222~regulation of | 2.20 × 10⁻⁵ | 1.23 | 0.11 | 0.0026 | 4.21 × 10⁻⁴ |
| metabolic process | | | | | |
| GO:0031325~positive | 2.63 × 10⁻⁵ | 1.75 | 0.13 | 0.0031 | 5.02 × 10⁻⁴ |
| regulation of cellular | | | | | |
| metabolic process | | | | | |
| GO:0042981~regulation of | 2.64 × 10⁻⁵ | 1.64 | 0.13 | 0.0030 | 5.05 × 10⁻⁴ |
| apoptosis | | | | | |
| GO:0009893~positive | 2.86 × 10⁻⁵ | 1.72 | 0.14 | 0.0032 | 5.47 × 10⁻⁴ |
| regulation of metabolic | | | | | |
| process | | | | | |
| GO:0031323~regulation of | 2.88 × 10⁻⁵ | 1.24 | 0.14 | 0.0031 | 5.50 × 10⁻⁴ |
| cellular metabolic process | | | | | |
| GO:0000819~sister | 2.91 × 10⁻⁵ | 4.55 | 0.14 | 0.0031 | 5.56 × 10⁻⁴ |
| chromatid segregation | | | | | |
| GO:0006953~acute-phase | 2.91 × 10⁻⁵ | 4.55 | 0.14 | 0.0031 | 5.56 × 10⁻⁴ |
| response | | | | | |
| GO:0051325~interphase | 3.92 × 10⁻⁵ | 2.72 | 0.19 | 0.0040 | 7.50 × 10⁻⁴ |
| GO:0012501~programmed | 4.18 × 10⁻⁵ | 1.50 | 0.20 | 0.0042 | 8.00 × 10⁻⁴ |
| cell death | | | | | |
| GO:0006915~apoptosis | 4.77 × 10⁻⁵ | 1.50 | 0.22 | 0.0047 | 9.13 × 10⁻⁴ |
| GO:0010468~regulation of | 5.16 × 10⁻ [symbol] | 1.23 | 0.24 | 0.0050 | 9.87 × 10⁻⁴ |
| gene expression | | | | | |
| GO:0006259~DNA metabolic | 9.88 × 10⁻⁵ | 1.45 | 0.41 | 0.0094 | 1.89 × 10⁻³ |
| process | | | | | |
| GO:0043170~macromolecule | 1.11 × 10⁻⁴ | 1.11 | 0.44 | 0.010 | 2.11 × 10⁻³ |
| metabolic process | | | | | |
| GO:0043065~positive | 1.13 × 10⁻⁴ | 1.91 | 0.45 | 0.010 | 2.15 × 10⁻³ |
| regulation of apoptosis | | | | | |
| GO:0045941~positive | 1.14 × 10⁻⁴ | 1.79 | 0.45 | 0.010 | 2.17 × 10⁻³ |
| regulation of transcription | | | | | |
| GO:0042107~cytokine | 1.19 × 10⁻⁴ | 2.87 | 0.47 | 0.011 | 2.28 × 10⁻³ |
| metabolic process | | | | | |
| GO:0045935~positve | 1.22 × 10⁻⁴ | 1.77 | 0.47 | 0.011 | 2.32 × 10⁻³ |
| regulation of nucleobase, | | | | | |
| nucleoside, nucleotide | | | | | |
| and nucleic acid | | | | | |
| metabolic process | | | | | |
| GO:0043068~positve | 1.33 × 10⁻⁴ | 1.89 | 0.50 | 0.011 | 2.55 × 10⁻³ |
| regulation of programmed | | | | | |
| cell death | | | | | |
| GO:0043412~biopolymer | 1.36 × 10⁻⁴ | 1.28 | 0.51 | 0.011 | 2.61 × 10⁻³ |
| modification | | | | | |
| GO:0006917~induction of | 1.42 × 10⁻⁴ | 1.99 | 0.53 | 0.012 | 2.71 × 10⁻³ |
| apoptosis | | | | | |
| GO:0051329~interphase of | 1.52 × 10⁻⁴ | 2.64 | 0.55 | 0.012 | 2.90 × 10⁻³ |
| mitotic cell cycle | | | | | |
| GO:0006464~protein | 1.54 × 10⁻⁴ | 1.28 | 0.56 | 0.012 | 2.94 × 10⁻³ |
| modification process | | | | | |
| GO:0012502~induction of | 1.55 × 10⁻⁴ | 1.98 | 0.56 | 0.012 | 2.97 × 10⁻³ |
| programmed cell death | | | | | |
| GO:0019219~regulation of | 2.01 × 10⁻⁴ | 1.22 | 0.65 | 0.016 | 3.84 × 10⁻³ |
| necleobase, nucleoside, | | | | | |
| nucleotide and nucleic | | | | | |
| acid metabolic process | | | | | |
| GO:0006355~regulation of | 2.03 × 10⁻⁴ | 1.23 | 0.66 | 0.016 | 3.87 × 10⁻³ |
| transcription, DNA- | | | | | |
| dependent | | | | | |
| GO:0006817~phosphate | 2.04 × 10⁻⁴ | 2.58 | 0.66 | 0.015 | 3.89 × 10⁻³ |
| transport | | | | | |
| GO:0030098~lymphocyte | 2.32 × 10⁻⁴ | 2.73 | 0.70 | 0.017 | 4.42 × 10⁻³ |
| differentiation | | | | | |
| GO:0002521~leukocyte | 2.55 × 10⁻⁴ | 2.40 | 0.74 | 0.019 | 4.87 × 10⁻³ |
| differentiation | | | | | |
| GO:0048729~tissue | 2.71 × 10⁻⁴ | 2.69 | 0.76 | 0.020 | 5.17 × 10⁻³ |
| morphogenesis | | | | | |
| GO:0043687~post- | 3.01 × 10⁻⁴ | 1.30 | 0.79 | 0.021 | 5.74 × 10⁻³ |
| translational protein | | | | | |
| modification | | | | | |
| GO:0031324~negative | 3.05 × 10⁻⁴ | 1.66 | 0.80 | 0.021 | 5.83 × 10⁻³ |
| regulation of cellular | | | | | |
| metabolic process | | | | | |
| GO:0015698~inorganic | 3.28 × 10⁻⁴ | 2.10 | 0.82 | 0.023 | 6.25 × 10⁻³ |
| anion transport | | | | | |
| GO:0042089~cytokine | 3.34 × 10⁻ | 2.75 | 0.83 | 0.023 | 6.37 × 10⁻³ |
| biosynthetic process | | | | | |
| GO:0007242~intracellular | 3.69 × 10⁻⁴ | 1.30 | 0.86 | 0.025 | 7.03 × 10⁻³ |
| signaling cascade | | | | | |
| GO:0000075~cell cycle | 4.20 × 10⁻⁴ | 2.93 | 0.89 | 0.028 | 8.00 × 10⁻³ |
| checkpoint | | | | | |
| hsa01430: Cell | 6.49 × 10⁻⁴ | 2.04 | 0.12 | 0.063 | 0.0081 |
| Communication | | | | | |
| GO:0045449~regulation of | 4.31 × 10⁻⁴ | 1.21 | 0.90 | 0.028 | 8.22 × 10⁻³ |
| transcription | | | | | |
| GO:0006351~transcription, | 4.68 × 10⁻⁴ | 1.21 | 0.91 | 0.030 | 8.92 × 10⁻ |
| DNA-dependent | | | | | |
| GO:0045893~positive | 4.76 × 10⁻⁴ | 1.80 | 0.92 | 0.030 | 9.07 × 10⁻³ |
| regulation of transcription, | | | | | |
| DNA-dependent | | | | | |
| GO:0032774~RNA | 5.08 × 10⁻⁴ | 1.21 | 0.93 | 0.032 | 9.67 × 10⁻³ |
| biosynthetic process | | | | | |
| GO:0009605~response to | 5.53 × 10⁻⁴ | 1.47 | 0.95 | 0.034 | 1.05 × 10⁻² |
| external stimulus | | | | | |
| GO:0001775~cell activation | 5.64 × 10⁻⁴ | 1.83 | 0.95 | 0.035 | 1.07 × 10⁻² |
| GO:0006950~response to | 5.72 × 10⁻⁴ | 1.35 | 0.95 | 0.035 | 1.09 × 10⁻² |
| stress | | | | | |
| GO:0046649~lymphocyte | 5.75 × 10⁻⁴ | 1.97 | 0.95 | 0.035 | 1.09 × 10⁻² |
| activation | | | | | |
| GO:0050000~chromosome | 6.02 × 10⁻⁴ | 10.1 | 0.96 | 0.036 | 1.14 × 10⁻² |
| localization | | | | | |
| GO:0051303~establishment | 6.02 × 10⁻⁴ | 10.1 | 0.96 | 0.036 | 1.14 × 10⁻² |
| of chromosome | | | | | |
| localization | | | | | |
| GO:0006270~DNA | 6.50 × 10⁻⁴ | 3.91 | 0.97 | 0.038 | 1.24 × 10⁻² |
| replication initiation | | | | | |
| GO:0006350~transcription | 6.76 × 10⁻⁴ | 1.20 | 0.97 | 0.039 | 1.29 × 10⁻² |
| GO:0006325~establishment | 7.06 × 10⁻⁴ | 1.69 | 0.98 | 0.040 | 1.34 × 10⁻² |
| and/or maintenance of | | | | | |
| chromatin architecture | | | | | |
| GO:0031424~keratinization | 7.76 × 10⁻⁴ | 3.51 | 0.98 | 0.043 | 1.47 × 10⁻² |
| GO:0042035~regulation of | 8.20 × 10⁻⁴ | 2.76 | 0.99 | 0.045 | 1.56 × 10⁻² |
| cytokine biosynthetic | | | | | |
| process | | | | | |
| GO:0007346~regulation of | 8.38 × 10⁻⁴ | 3.79 | 0.99 | 0.046 | 1.59 × 10⁻² |
| progression through | | | | | |
| mitotic cell cycle | | | | | |
| GO:0040029~regulation of | 8.67 × 10⁻⁴ | 3.03 | 0.99 | 0.047 | 1.64 × 10⁻² |
| gene expression, | | | | | |
| epigenetic | | | | | |
| GO:0045934~negative | 8.90 × 10⁻⁴ | 1.66 | 0.99 | 0.048 | 1.69 × 10⁻² |
| regulation of nucleobase, | | | | | |
| nucleoside, nucleotide | | | | | |
| and nucleic acid | | | | | |
| metabolic process | | | | | |
| GO:0065009~regulation of a | 9.15 × 10⁻⁴ | 1.49 | 0.99 | 0.048 | 1.74 × 10⁻² |
| molecular function | | | | | |
| hsa04610: Complement and | 0.0014 | 2.47 | 0.25 | 0.090 | 0.018 |
| coagulation cascades | | | | | |
| GO:0048519~negative | 9.43 × 10⁻⁴ | 1.31 | 0.99 | 0.049 | 1.79 × 10⁻² |
| regulation of biological | | | | | |
| process | | | | | |
| GO:0009892~negative | 9.79 × 10⁻⁴ | 1.56 | 0.99 | 0.051 | 1.86 × 10⁻² |
| regulation of metabolic | | | | | |
| process | | | | | |
| GO:0006323~DNA | 0.0010 | 1.66 | 0.996 | 0.053 | 1.97 × 10⁻² |
| packaging | | | | | |
| GO:0006139~nucleobase, | 0.0011 | 1.143 | 0.997 | 0.055 | 2.04 × 10⁻² |
| nucleoside, nucleotide | | | | | |
| and nucleic acid | | | | | |
| metabolic process | | | | | |
| GO:0048523~negative | 0.0011 | 1.32 | 0.997 | 0.055 | 2.08 × 10⁻² |
| regulation of cellular | | | | | |
| process | | | | | |
| GO:0050790~regulation of | 0.0011 | 1.52 | 0.997 | 0.055 | 2.11 × 10⁻² |
| catalytic activity | | | | | |
| GO:0045859~regulation of | 0.0012 | 1.79 | 0.998 | 0.060 | 2.32 × 10⁻² |
| protein kinase activity | | | | | |
| GO:0042110~T cell | 0.0013 | 2.18 | 0.999 | 0.065 | 2.55 × 10⁻² |
| activation | | | | | |
| GO:0051338~regulation of | 0.0014 | 1.76 | 0.999 | 0.067 | 2.64 × 10⁻² |
| transferase activity | | | | | |
| GO:0007399~nervous | 0.0014 | 1.38 | 0.999 | 0.068 | 2.72 × 10⁻ |
| system development | | | | | |
| GO:0007010~cytoskeleton | 0.0016 | 1.48 | 0.9998 | 0.076 | 3.08 × 10⁻² |
| organization and | | | | | |
| biogenesis | | | | | |
| GO:0016481~negative | 0.0017 | 1.66 | 0.9998 | 0.077 | 3.13 × 10⁻² |
| regulation of transcription | | | | | |
| GO:0009889~regulation of | 0.0017 | 1.82 | 0.9999 | 0.078 | 3.21 × 10⁻² |
| biosynthetic process | | | | | |
| GO:0006333~chromatin | 0.0017 | 2.01 | 0.9999 | 0.079 | 3.27 × 10⁻² |
| assembly or disassembly | | | | | |
| GO:0006468~protein amino | 0.0018 | 1.39 | 0.9999 | 0.084 | 3.53 × 10⁻² |
| acid phosphorylation | | | | | |
| GO:0043549~regulation of | 0.0019 | 1.75 | 0.99995 | 0.084 | 3.56 × 10⁻² |
| kinase activity | | | | | |
| GO:0000085~G2 phase of | 0.0021 | 12.1 | 0.99998 | 0.092 | 3.94 × 10⁻² |
| mitotic cell cycle | | | | | |
| GO:0051319~G2 phase | 0.0021 | 12.1 | 0.99998 | 0.092 | 3.94 × 10⁻² |
| GO:0009611~response to | 0.0021 | 1.52 | 0.99998 | 0.091 | 3.94 × 10⁻² |
| wounding | | | | | |
| GO:0030217~T cell | 0.0021 | 2.91 | 0.99999 | 0.091 | 3.99 × 10⁻² |
| differentiation | | | | | |
| hsa04115: p53 signaling | 0.0034 | 2.35 | 0.50 | 0.16 | 0.042 |
| pathway | | | | | |
| GO:0006959~humoral | 0.0023 | 2.49 | 0.99999 | 0.097 | 4.27 × 10⁻² |
| immune respone | | | | | |
| h_extrinsicPathway: Extrinsic | 0.0032 | 5.18 | 0.65 | 0.65 | 0.043 |
| Prothrombin Activation | | | | | |
| Pathway | | | | | |
| GO:0048730~epidermis | 0.0024 | 2.72 | 0.999996 | 0.099 | 4.43 × 10⁻² |
| morphogenesis | | | | | |
| GO:0042094~interleukin-2 | 0.0024 | 4.71 | 0.999997 | 0.10 | 4.53 × 10⁻² |
| biosynthetic process | | | | | |
| GO:0016070~RNA metabolic | 0.0025 | 1.16 | 0.999998 | 0.10 | 4.68 × 10⁻² |
| process | | | | | |

[symbol]

indicates data missing or illegible when filed

TABLE 4
Curated Gene Sets Overexpressed by ccA Tumors

| Gene Set | p-value |
|---|---|
| 1_2_DICHLOROETHANE_DEGRADATION | 0.0248 |
| ADIP_VS_PREADIP_UP | 0.0186 |
| AGEING_KIDNEY_DN | 0.0394 |
| AGEING_KIDNEY_SPECIFIC_DN | 0.028 |
| AGEING_LYMPH_DN | 0.0186 |
| AGUIRRE_PANCREAS_CHR18 | 0.0432 |
| ASCORBATE_AND_ALDARATE_METABOLISM | 0.0248 |
| BCRABL_HL60_AFFY_UP | 0.0024 |
| BECKER_ESTROGEN_RESPONSIVE_SUBSET_2 | 0.0336 |
| BECKER_TAMOXIFEN_RESISTANT_DN | 0.0236 |
| BENZOATE_DEGRADATION_VIA_COA_LIGATION | 0.0272 |
| BETA_ALANINE_METABOLISM | 0.0134 |
| BETAOXIDATIONPATHWAY | 0.0028 |
| BLOOD_GROUP_GLYCOLIPID_BIOSYNTHESIS_NEOLACTOSERIES | 0.0012 |
| BRCA_ER_POS | 0.0262 |
| BRCA_PROGNOSIS_POS | 0.0326 |
| BRCA1_OVEREXP_PROSTATE_DN | 0.0198 |
| BRCA1_OVEREXP_UP | 0.0286 |
| BUTANOATE_METABOLISM | 0.0108 |
| CALRES_RHESUS_DN | 0.0054 |
| CAPROLACTAM_DEGRADATION | 0.049 |
| CITED1_KO_HET_DN | 0.0492 |
| CITED1_KO_WT_DN | 0.0234 |
| CMV_HCMV_TIMECOURSE_16HRS_DN | 0.021 |
| CYANOAMINO_ACID_METABOLISM | 0.0064 |
| ERBB3PATHWAY | 0.0458 |
| ET743PT650_COLONCA_DN | 0.0418 |
| FALT_BCLL_DN | 0.0306 |
| FALT_BCLL_IG_MUTATED_VS_WT_DN | 0.0226 |
| FATTY_ACID_BIOSYNTHESIS_PATH_2 | 0.0024 |
| FATTY_ACID_DEGRADATION | 0.0082 |
| FATTY_ACID_SYNTHESIS | 0.0176 |
| FLECHNER_KIDNEY_TRANSPLANT_REJECTION_DN | 0.0068 |
| FLECHNER_KIDNEY_TRANSPLANT_WELL_UP | 0.0368 |
| GAMMA_ESR_WS_UNREG | 0.0112 |
| GLYCOSPHINGOLIPID_METABOLISM | 0.0054 |
| HDACI_COLON_CLUSTER7 | 0.0462 |
| HDACI_COLON_CLUSTER8 | 0.0204 |
| HDACI_COLON_TSA24HRS_DN | 0.0358 |
| HEARTFAILURE_ATRIA_DN | 0.0364 |
| HEATSHOCK_OLD_UP | 0.0148 |
| HIPPOCAMPUS_DEVELOPMENT_NEONATAL | 0.0494 |
| HISTIDINE_METABOLISM | 0.0138 |
| HSA00053_ASCORBATE_AND_ALDARATE_METABOLISM | 0.0236 |
| HSA00062_FATTY_ACID_ELONGATION_IN_MITOCHONDRIA | 0.0104 |
| HSA00071_FATTY_ACID_METABOLISM | 0.0158 |
| HSA00120_BILE_ACID_BIOSYNTHESIS | 0.0276 |
| HSA00280_VALINE_LEUCINE_AND_ISOLEUCINE_DEGRADATION | 0.0174 |
| HSA00310_LYSINE_DEGRADATION | 0.026 |
| HSA00340_HISTIDINE_METABOLISM | 0.0226 |
| HSA00380_TRYPTOPHAN_METABOLISM | 0.0168 |
| HSA00410_BETA_ALANINE_METABOLISM | 0.034 |
| HSA00460_CYANOAMINO_ACID_METABOLISM | 0.0178 |
| HSA00600_SPHINGOLIPID_METABOLISM | 0.016 |
| HSA00602_GLYCOSPHINGOLIPID_BIOSYNTHESIS_NEO_LACTOSERIES | 0.0384 |
| HSA00625_TETRACHLOROETHENE_DEGRADATION | 0.0236 |
| HSA00640_PROPANOATE_METABOLISM | 0.024 |
| HSA00650_BUTANOATE_METABOLISM | 0.043 |
| HSA00680_METHANE_METABOLISM | 0.0352 |
| HSA00903_LIMONENE_AND_PINENE_DEGRADATION | 0.0316 |
| HSA01031_GLYCAN_STRUCTURES_BIOSYNTHESIS_2 | 0.0152 |
| HYPOPHYSECTOMY_RAT_DN | 0.041 |
| HYPOPHYSECTOMY_RAT_UP | 0.0348 |
| HYPOXIA_FIBRO_UP | 0.0402 |
| HYPOXIA_NORMAL_UP | 0.0144 |
| HYPOXIA_REG_UP | 0.035 |
| IDX_TSA_DN_CLUSTER4 | 0.0038 |
| IDX_TSA_UP_CLUSTER6 | 0.0226 |
| LEE_CIP_UP | 0.018 |
| LEPTINPATHWAY | 0.0382 |
| LI_FETAL_VS_WT_KIDNEY_UP | 0.0038 |
| LIMONENE_AND_PINENE_DEGRADATION | 0.0118 |
| LIZUKA_G2_GR_G3 | 0.016 |
| LYSINE_DEGRADATION | 0.0142 |
| MENSE_HYPOXIA_APOPTOSIS_GENES | 0.003 |
| MENSE_HYPOXIA_UP | 0.0382 |
| METHANE_METABOLISM | 0.045 |
| MITOCHONDRIAL_FATTY_ACID_BETAOXIDATION | 0.0064 |
| P21_ANY_UP | 0.0434 |
| PGC | 0.0406 |
| PROPANOATE_METABOLISM | 0.006 |
| PYRUVATE_METABOLISM | 0.0226 |
| ROME_INSULIN_2F_UP | 0.0318 |
| ROSS_FAB_M7 | 0.0362 |
| SANA_IFNG_ENDOTHELIAL_DN | 0.043 |
| SMITH_HCV_INDUCED_HCC_UP | 0.0126 |
| SMITH_HTERT_DN | 0.0094 |
| SYNTHESIS_AND_DEGRADATION_OF_KETONE_BODIES | 0.0364 |
| TZD_ADIP_DN | 0.0436 |
| UVB_NHEK2_DN | 0.0486 |
| VALINE_LEUCINE_AND_ISOLEUCINE_DEGRADATION | 0.0058 |
| VENTRICLES_UP | 0.0202 |
| WALKER_MM_SNP_DIFF | 0.0492 |
| ZHAN_MM_CD1_VS_CD2_UP | 0.0338 |

TABLE 5
Curated Gene Sets Overexpressed by ccB Tumors

| Gene Set | p-value |
|---|---|
| SARCOMAS_LEIOMYOSARCOMA_UP | 0.002 |
| TSADAC_PANC50_UP | 0.0042 |
| SHEPARD_BMYB_MORPHOLINO_DN | 0.0106 |
| DAC_PANC50_UP | 0.0118 |
| SHEPARD_GENES_COMMON_BW_CB_MO | 0.0124 |
| IL6_SCAR_FIBRO_UP | 0.0138 |
| TGFBETA_C4_UP | 0.014 |
| DNMT1_KO_DN | 0.0154 |
| SARCOMAS_LIPOSARCOMA_DN | 0.0168 |
| CELL_PROLIFERATION | 0.0176 |
| SHEPARD_CELL_PROLIFERATION | 0.0176 |
| MIDDLEAGE_DN | 0.0198 |
| ADIP_DIFF_CLUSTER4 | 0.0202 |
| MUNSHI_MM_VS_PCS_DN | 0.0204 |
| BECKER_CANCER_ASSOCIATED_SUBSET_1 | 0.0226 |
| AS3_HEK293_DN | 0.0252 |
| CITED1_KO_WT_UP | 0.0254 |
| SRCRPTPPATHWAY | 0.0268 |
| TNFALPHA_ADIP_UP | 0.0268 |
| SHEPARD_CRASH_AND_BURN_MUT_VS_WT_DN | 0.027 |
| LEI_MYB_REGULATED_GENES | 0.027 |
| BCL2_FAMILY_AND_REG_NETWORK | 0.0272 |
| WNT_TARGETS | 0.0278 |
| CROONQUIST_IL6_RAS_DN | 0.03 |
| HUMAN_TISSUE_TESTIS | 0.0302 |
| GAY_YY1_DN | 0.0312 |
| IGLESIAS_E2FMINUS_DN | 0.0312 |
| ST_T_CELL_SIGNAL_TRANSDUCTION | 0.0316 |
| CIS_XPC_UP | 0.0328 |
| HG_PROGERIA_DN | 0.0332 |
| JECHLINGER_EMT_UP | 0.0334 |
| SA_FAS_SIGNALING | 0.0368 |
| BRENTANI_DNA_METHYLATION_AND_MODIFICATION | 0.0374 |
| O_GLYCAN_BIOSYNTHESIS | 0.0376 |
| HOFFMANN_BIVSBII_BI_TABLE2 | 0.0376 |
| OLDAGE_DN | 0.0378 |
| POD1_KO_UP | 0.0382 |
| TPA_SENS_EARLY_DN | 0.0382 |
| DAC_PANC_UP | 0.0382 |
| PEART_HISTONE_DN | 0.0404 |
| HSA04115_P53_SIGNALING_PATHWAY | 0.0412 |
| LE_MYELIN_UP | 0.0414 |
| IDX_TSA_UP_CLUSTER2 | 0.0416 |
| IONPATHWAY | 0.0424 |
| ADIP_HUMAN_DN | 0.0426 |
| BRCA_ER_NEG | 0.0432 |
| PEPIPATHWAY | 0.0434 |
| P21_P53_ANY_DN | 0.0436 |
| ELONGINA_KO_DN | 0.044 |
| HUMAN_TISSUE_PLACENTA | 0.0444 |
| PARP_KO_DN | 0.045 |
| P21_P53_EARLY_DN | 0.0468 |
| BCNU_GLIOMA_MGMT_24HRS_DN | 0.047 |
| XU_CBP_UP | 0.0476 |
| HSA04610_COMPLEMENT_AND_COAGULATION_CASCADES | 0.0478 |
| P21_P53_MIDDLE_DN | 0.048 |
| EMT_UP | 0.0488 |
| MTX_RES_XENOGRAFTS_UP | 0.0494 |

### Example 3

### Delineation of a Gene Set to Stratify ccRCC into ccA and ccB

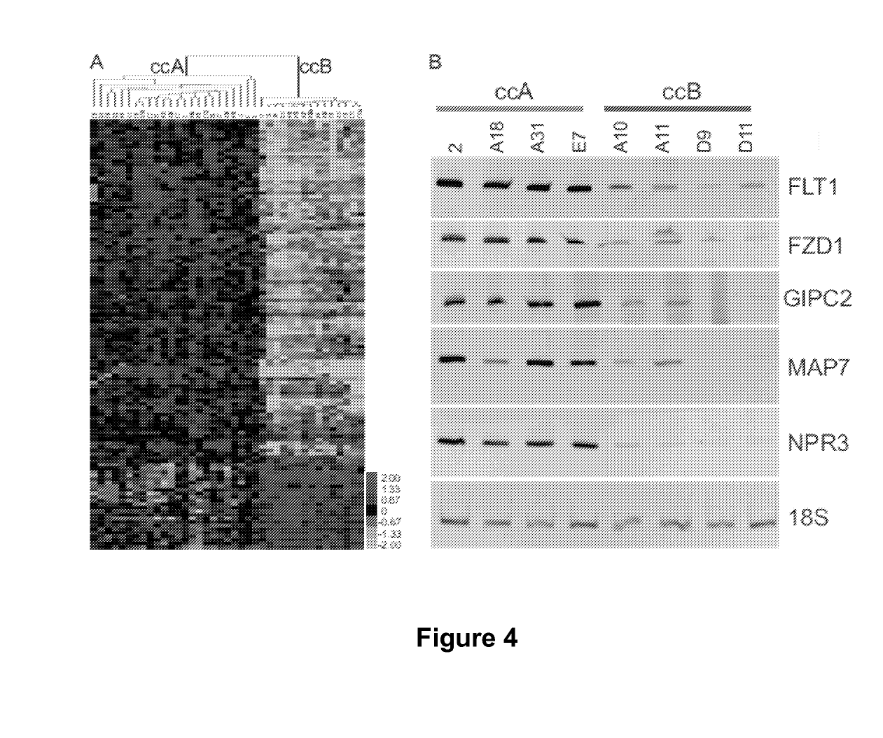

To identify a profile that could accurately identify ccA and ccB tumors, logical analysis of data (LAD), which uses pattern recognition and supervised learning to identify key discriminating elements and has been successfully implemented in several biomedical studies (Alexe et al., 2006; Dalgin et al., 2007; Reddy et al., 2008) was employed. Using the core ccA and ccB tumors, LAD patterns were identified and validated. Using these patterns, 120 probes were identified that corresponded to 110 genes valuable for cluster assignment (FIG. 4A, Table 6, and Table 7). The LAD model (Tables 8 and 9) was applied to the 12 non-core samples from the original analysis, and predicted cluster membership for 11 samples: 8 ccA and 3 ccB (Table 10).

TABLE 6
LAD Gene Set*

| Subtype | GENBANK ® Acc. No.¹ | Symbol | Fold change² |
|---|---|---|---|
| ccA | NM_006111 | ACAA2 | 4.159 |
| ccA | NM_001608 | ACADL | 2.712 |
| ccA | NM_000019 | ACAT1 | 2.795 |
| ccA | NM_032360 | ACBD6 | 1.516 |
| ccA | NM_001122 | ADFP | 3.951 |
| ccA | NM_006796 | AFG3L2 | 2.247 |
| ccA | NM_000382 | ALDH3A2 | 3.327 |
| ccA | NM_173039 | AQP11 | 2.899 |
| ccA | NM_000047 | ARSE | 3.24 |
| ccA | NM_006876 | B3GNT6 | 2.41 |
| ccA | NM_033177 | BAT4 | 1.706 |
| ccA | NM_004331 | BNIP3L | 2.503 |
| ccA | NM_022761 | C11orf1 | 2.47 |
| ccA | NM_020456 | C13orf1 | 2.483 |
| ccA | NM_020456 | C13orf1 | 2.081 |
| ccA | NM_018112 | C9orf87 | 4.427 |
| ccA | NM_152434 | CWF19L2 | 1.598 |
| ccA | AB082528 | DNCH2 | 2.023 |
| ccA | NM_016025 | DREV1 | 2.161 |
| ccA | NM_153682 | DSCR5 | 2.553 |
| ccA | NM_024693 | ECHDC3 | 3.653 |
| ccA | NM_015252 | EHBP1 | 2.003 |
| ccA | NM_001984 | ESD | 1.661 |
| ccA | NM_031208 | FAHD1 | 2.671 |
| ccA | NM_138369 | FAM44B | 2.147 |
| ccA | NM_205857 | FBI4 | 2.75 |
| ccA | NM_205857 | FBI4 | 2.02 |
| ccA | NM_018359 | FLJ11200 | 2.149 |
| ccA | NM_024603 | FLJ11588 | 2.2 |
| ccA | NM_024584 | FLJ13646 | 1.997 |
| ccA | NM_024563 | FLJ14054 | 9.81 |
| ccA | NM_024709 | FLJ14146 | 3.067 |
| ccA | NM_022460 | FLJ14249 | 2.159 |
| ccA | NM_022460 | FLJ14249 | 1.89 |
| ccA | NM_022918 | FLJ22104 | 3.108 |
| ccA | NM_022918 | FLJ22104 | 2.885 |
| ccA | AK125261 | FLJ23834 | 2.499 |
| ccA | CR593388 | FLT1 | 3.07 |
| ccA | NM_003505 | FZD1 | 3.116 |
| ccA | NM_003774 | GALNT4 | 1.804 |
| ccA | NM_000163 | GHR | 3.943 |
| ccA | NM_017655 | GIPC2 | 5.447 |
| ccA | NM_017655 | GIPC2 | 4.163 |
| ccA | NM_015700 | HIRIP5 | 2 |
| ccA | NM_002141 | HOXA4 | 3.165 |
| ccA | NM_017409 | HOXC10 | 2.467 |
| ccA | NM_014278 | HSPA4L | 2.339 |
| ccA | NM_000210 | ITGA6 | 2.15 |
| ccA | NM_005472 | KCNE3 | 2.633 |
| ccA | NM_006036 | KIAA0436 | 2.394 |
| ccA | AB028966 | KIAA1043 | 1.876 |
| ccA | AK092338 | KIAA1648 | 1.897 |
| ccA | NM_015344 | LEPROTL1 | 2.579 |
| ccA | NM_138787 | LOC119710 | 2.167 |
| ccA | NM_138809 | LOC134147 | 3.346 |
| ccA | NM_020422 | LOC57146 | 2.685 |
| ccA | NM_181705 | LOC90624 | 2.03 |
| ccA | NM_000898 | MAOB | 3.677 |
| ccA | NM_003980 | MAP7 | 3.598 |
| ccA | NM_016835 | MAPT | 4.959 |
| ccA | NM_016835 | MAPT | 3.428 |
| ccA | NM_144611 | MGC32124 | 1.938 |
| ccA | NM_145036 | MGC33887 | 2.095 |
| ccA | NM_181515 | MRPL21 | 1.605 |
| ccA | NM_018092 | NETO2 | 4.082 |
| ccA | NM_004808 | NMT2 | 2.369 |
| ccA | NM_000908 | NPR3 | 7.48 |
| ccA | NM_000908 | NPR3 | 7.362 |
| ccA | NM_177533 | NUDT14 | 2.408 |
| ccA | NM_080597 | OSBPL1A | 2.354 |
| ccA | NM_025208 | PDGFD | 3.585 |
| ccA | NM_006214 | PHYH | 2.62 |
| ccA | NM_002676 | PMM1 | 1.897 |
| ccA | NM_006252 | PRKAA2 | 2.832 |
| ccA | NM_014039 | PTD012 | 3.632 |
| ccA | CR611332 | PURA | 2.179 |
| ccA | NM_175623 | RAB3IP | 3.301 |
| ccA | NM_002139 | RBMX | 1.558 |
| ccA | NM_002906 | RDX | 1.988 |
| ccA | NM_001145 | RNASE4 | 3.083 |
| ccA | AF440762 | SETP8 | 2.232 |
| ccA | NM_004170 | SLC1A1 | 4.695 |
| ccA | NM_018158 | SLC4A1AP | 1.339 |
| ccA | NM_003759 | SLC4A4 | 3.022 |
| ccA | NM_003932 | ST13 | 1.644 |
| ccA | NM_018401 | STK32B | 3.508 |
| ccA | NM_003196 | TCEA3 | 2.726 |
| ccA | NM_003196 | TCEA3 | 2.904 |
| ccA | NM_003196 | TCEA3 | 2.967 |
| ccA | NM_000355 | TCN2 | 2.657 |
| ccA | NM_053000 | TIGA1 | 3.288 |
| ccA | NM_003265 | TLR3 | 4.409 |
| ccA | NM_001004125 | TUSC1 | 2.817 |
| ccA | NM_001004125 | TUSC1 | 2.883 |
| ccA | NM_139312 | YME1L1 | 1.46 |
| ccA | NM_152444 | ZADH1 | 3.082 |
| ccB | NM_170697 | ALDH1A2 | 0.333 |
| ccB | NM_006594 | AP4B1 | 0.624 |
| ccB | NM_198540 | B3GALT7 | 0.456 |
| ccB | NM_138639 | BCL2L12 | 0.609 |
| ccB | NM_016606 | C5orf19 | 0.262 |
| ccB | NM_001793 | CDH3 | 0.201 |
| ccB | NM_016229 | CYB5R2 | 0.408 |
| ccB | AK074447 | FLJ23867 | 0.447 |
| ccB | AK021777 | GALNT10 | 0.356 |
| ccB | BQ188318 | IMP-2 | 0.245 |
| ccB | NM_004823 | KCNK6 | 0.551 |
| ccB | NM_002250 | KCNN4 | 0.35 |
| ccB | NM_003833 | MATN4 | 0.317 |
| ccB | NM_152789 | MGC40405 | 0.499 |
| ccB | NM_080678 | NCE2 | 0.618 |
| ccB | NM_006993 | NPM3 | 0.517 |
| ccB | NM_006512 | SAA4 | 0.293 |
| ccB | NM_003064 | SLPI | 0.19 |
| ccB | NM_032872 | SYTL1 | 0.348 |
| ccB | NM_003290 | TPM4 | 0.469 |
| ccB | NM_015644 | TTLL3 | 0.415 |
| ccB | NM_021147 | UNG2 | 0.283 |
| ccB | NM_003363 | USP4 | 0.507 |
| ccB | AK123473 | ZNF292 | 0.303 |

*Probes identified through logical analysis of data (LAD) to discriminate between ccA and ccB subtypes. All probes were significant at t-test p < 0.000001.
¹GENBANK ® Accession Numbers correspond to nucleic acid sequences, and each GENBANK ® database entry is incorporated by reference in its entirety, including all annotations.
²Fold change was calculated as ccA/ccB. Full names, Unigene cluster Id. numbers, and associated GENBANK ® Accession Numbers are shown in Table 7 below.

TABLE 7
LAD Probes that Distinguish between Subtypes ccA and ccB

| | | Unigene | GENBANK ® |
|---|---|---|---|
| Symbol | Description | ClusterID* | Acc. No. |
| ACAA2 | Acetyl-Coenzyme A | Hs.200136 | NM_006111 |
| | acyltransferase 2 | | |
| | (mitochondrial 3-oxoacyl- | | |
| | Coenzyme A thiolase) | | |
| ACADL | Acyl-Coenzyme A | Hs.471277 | NM_001608 |
| | dehydrogenase, long | | |
| | chain | | |
| ACAT1 | Acetyl-Coenzyme A | Hs.232375 | NM_000019 |
| | acetyltransferase 1 | | |
| | (acetoacetyl Coenzyme A | | |
| | thiolase) | | |
| ACBD6 | Acyl-Coenzyme A binding | Hs.200051 | NM_032360 |
| | domain containing 6 | | |
| ADFP | Adipose differentiation- | Hs.3416 | NM_001122 |
| | related protein | | |
| AFG3L2 | AFG3 ATPase family gene | Hs.528996 | NM_006796 |
| | 3-like 2 (yeast) | | |
| ALDH1A2 | Aldehyde dehydrogenase 1 | Hs.435689 | NM_170697 |
| | family, member A2 | | |
| ALDH3A2 | Aldehyde dehydrogenase 3 | Hs.499886 | NM_000382 |
| | family, member A2 | | |
| AP4B1 | Adaptor-related protein | Hs.515048 | NM_006594 |
| | complex 4, beta 1 subunit | | |
| AQP11 | Aquaporin 11 | Hs.503345 | NM_173039 |
| ARSE | Arylsulfatase E | Hs.386975 | NM_000047 |
| | (chondrodysplasia | | |
| | punctata 1) | | |
| B3GALT7 | UDP-Gal: betaGal beta 1,3- | Hs.441681 | NM_198540 |
| | galactosyltransferase | | |
| | polypeptide 7 | | |
| B3GNT6 | UDP-GlcNAc: betaGal beta- | Hs.8526 | NM_006876 |
| | 1,3-N-acetylglucosaminyl- | | |
| | transferase 6 | | |
| BAT4 | HLA-B associated transcript 4 | Hs.247478 | NM_033177 |
| BCL2L12 | BCL2-like 12 (proline rich) | Hs.289052 | NM_138639 |
| BNIP3L | BCL2/adenovirus E1B | Hs.131226 | NM_004331 |
| | 19 kDa interacting protein | | |
| | 3-like | | |
| C11orf1 | Chromosome 11 open | Hs.17546 | NM_022761 |
| | reading frame 1 | | |
| C13orf1 | Chromosome 13 open | Hs.44235 | NM_020456 |
| | reading frame 1 | | |
| C13orf1 | Chromosome 13 open | Hs.44235 | NM_020456 |
| | reading frame 1 | | |
| C5orf19 | Chromosome 5 open | Hs.416090 | NM_016606 |
| | reading frame 19 | | |
| C9orf87 | Chromosome 9 open | Hs.411925 | NM_018112 |
| | reading frame 87 | | |
| CDH3 | Cadherin 3, type 1, P- | Hs.191842 | NM_001793 |
| | cadherin (placental) | | |
| CWF19L2 | CWF19-like 2, cell cycle | Hs.212140 | NM_152434 |
| | control (S. pombe) | | |
| CYB5R2 | Cytochrome b5 reductase | Hs.414362 | NM_016229 |
| | b5R.2 | | |
| DNCH2 | Dynein, cytoplasmic, heavy | Hs.503721 | AB082528 |
| | polypeptide 2 | | |
| DREV1 | DORA reverse strand | Hs.279583 | NM_016025 |
| | protein 1 | | |
| DSCR5 | Down syndrome critical | Hs.408790 | NM_153682 |
| | region gene 5 | | |
| ECHDC3 | Enoyl Coenzyme A | Hs.22242 | NM_024693 |
| | hydratase domain | | |
| | containing 3 | | |
| EHBP1 | EH domain binding protein 1 | Hs.271667 | NM_015252 |
| ESD | Esterase | Hs.432491 | NM_001984 |
| | D/formylglutathione | | |
| | hydrolase | | |
| FAHD1 | Hydroxyacylglutathione | Hs.513265 | NM_031208 |
| | hydrolase | | |
| FAM44B | Family with sequence | Hs.425091 | NM_138369 |
| | similarity 44, member B | | |
| FBI4 | FBI4 protein | Hs.46730 | NM_205857 |
| FBI4 | FBI4 protein | Hs.46730 | NM_205857 |
| FLJ11200 | Hypothetical protein | Hs.368022 | NM_018359 |
| | FLJ11200 | | |
| FLJ11588 | Hypothetical protein | Hs.475348 | NM_024603 |
| | FLJ11588 | | |
| FLJ13646 | Hypothetical protein | Hs.21081 | NM_024584 |
| | FLJ13646 | | |
| FLJ14054 | Hypothetical protein | Hs.13528 | NM_024563 |
| | FLJ14054 | | |
| FLJ14146 | Hypothetical protein | Hs.519839 | NM_024709 |
| | FLJ14146 | | |
| FLJ14249 | HS1-binding protein 3 | Hs.531785 | NM_022460 |
| FLJ14249 | HS1-binding protein 3 | Hs.531785 | NM_022460 |
| FLJ22104 | Hypothetical protein | Hs.188591 | NM_022918 |
| | FLJ22104 | | |
| FLJ22104 | Hypothetical protein | Hs.188591 | NM_022918 |
| | FLJ22104 | | |
| FLJ23834 | Hypothetical protein | Hs.202120 | AK125261 |
| | FLJ23834 | | |
| FLJ23867 | Hypothetical protein | Hs.447969 | AK074447 |
| | FLJ23867 | | |
| FLT1 | Fms-related tyrosine kinase | Hs.507621 | CR593388 |
| | 1 (vascular endothelial | | |
| | growth factor/vascular | | |
| | permeability factor | | |
| | receptor) | | |
| FZD1 | Frizzled homolog 1 | Hs.94234 | NM_003505 |
| | (Drosophila) | | |
| GALNT10 | UDP-N-acetyl-alpha-D- | Hs.34421 | AK021777 |
| | galactosamine: polypeptide | | |
| | N-acetylgalactosaminyl- | | |
| | transferase10 (GalNAc-T10) | | |
| GALNT4 | UDP-N-acetyl-alpha-D- | Hs.534374 | NM_003774 |
| | galactosamine: polypeptide | | |
| | N-acetylgalactosaminyl- | | |
| | transferase 4 (GalNAc-T4) | | |
| GHR | Growth hormone receptor | Hs.125180 | NM_000163 |
| GIPC2 | PDZ domain protein GIPC2 | Hs.13852 | NM_017655 |
| GIPC2 | PDZ domain protein GIPC2 | Hs.13852 | NM_017655 |
| HIRIP5 | HIRA interacting protein 5 | Hs.430439 | NM_015700 |
| HOXA4 | Homeo box A4 | Hs.77637 | NM_002141 |
| HOXC10 | Homeo box C10 | Hs.44276 | NM_017409 |
| HSPA4L | Heat shock 70 kDa protein 4- | Hs.135554 | NM_014278 |
| | like | | |
| IMP-2 | IGF-II mRNA-binding protein 2 | Hs.35354 | BQ188318 |
| ITGA6 | Integrin, alpha 6 | Hs.133397 | NM_000210 |
| KCNE3 | Potassium voltage-gated | Hs.523899 | NM_005472 |
| | channel, lsk-related | | |
| | family, member 3 | | |
| KCNK6 | Potassium channel, | Hs.240395 | NM_004823 |
| | subfamily K, member 6 | | |
| KCNN4 | Potassium | Hs.10082 | NM_002250 |
| | intermediate/small | | |
| | conductance calcium- | | |
| | activated channel, | | |
| | subfamily N, member 4 | | |
| KIAA0436 | Putative prolyl | Hs.110 | NM_006036 |
| | oligopeptidase | | |
| KIAA1043 | KIAA1043 protein | Hs.387856 | AB028966 |
| KIAA1648 | KIAA1648 protein | Hs.348799 | AK092338 |
| LEPROTL1 | Leptin receptor overlapping | Hs.146585 | NM_015344 |
| | transcript-like 1 | | |
| LOC119710 | Hypothetical protein | Hs.406726 | NM_138787 |
| | BC009561 | | |
| LOC134147 | Hypothetical protein | Hs.192586 | NM_138809 |
| | BC001573 | | |
| LOC57146 | Promethin | Hs.258212 | NM_020422 |
| LOC90624 | Hypothetical protein | Hs.115467 | NM_181705 |
| | LOC90624 | | |
| MAOB | Monoamine oxidase B | Hs.46732 | NM_000898 |
| MAP7 | Microtubule-associated | Hs.486548 | NM_003980 |
| | protein 7 | | |
| MAPT | Microtubule-associated | Hs.101174 | NM_016835 |
| | protein tau | | |
| MAPT | Microtubule-associated | Hs.101174 | NM_016835 |
| | protein tau | | |
| MATN4 | Matrilin 4 | Hs.278489 | NM_003833 |
| MGC32124 | Hypothetical protein | Hs.513871 | NM_144611 |
| | MGC32124 | | |
| MGC33887 | Hypothetical protein | Hs.408676 | NM_145036 |
| | MGC33887 | | |
| MGC40405 | Hypothetical protein | Hs.489105 | NM_152789 |
| | MGC40405 | | |
| MRPL21 | Mitochondrial ribosomal | Hs.503047 | NM_181515 |
| | protein L21 | | |
| NCE2 | NEDD8-conjugating enzyme | Hs.471785 | NM_080678 |
| NETO2 | Neuropilin (NRP) and tolloid | Hs.444046 | NM_018092 |
| | (TLL)-like 2 | | |
| NMT2 | N-myristoyltransferase 2 | Hs.60339 | NM_004808 |
| NPM3 | Nucleophosmin/nucleoplasmin, 3 | Hs.90691 | NM_006993 |
| NPR3 | Natriuretic peptide receptor | Hs.237028 | NM_000908 |
| | C/guanylate cyclase C | | |
| | (atrionatriuretic peptide | | |
| | receptor C) | | |
| NPR3 | Natriuretic peptide receptor | Hs.237028 | NM_000908 |
| | C/guanylate cyclase C | | |
| | (atrionatriuretic peptide | | |
| | receptor C) | | |
| NUDT14 | Nudix (nucleoside | Hs.526432 | NM_177533 |
| | diphosphate linked | | |
| | moiety X)-type motif 14 | | |
| OSBPL1A | Oxysterol binding protein- | Hs.370725 | NM_080597 |
| | like 1A | | |
| PDGFD | DNA-damage inducible | Hs.352298 | NM_025208 |
| | protein 1 | | |
| PHYH | Phytanoyl-CoA hydroxylase | Hs.498732 | NM_006214 |
| | (Refsum disease) | | |
| PMM1 | Phosphomannomutase 1 | Hs.75835 | NM_002676 |
| PRKAA2 | Protein kinase, AMP- | Hs.256067 | NM_006252 |
| | activated, alpha 2 | | |
| | catalytic subunit | | |
| PTD012 | PTD012 protein | Hs.8360 | NM_014039 |
| PURA | Purine-rich element binding | Hs.443121 | CR611332 |
| | protein A | | |
| RAB3IP | RAB3A interacting protein | Hs.258209 | NM_175623 |
| | (rabin3) | | |
| RBMX | RNA binding motif protein, | Hs.380118 | NM_002139 |
| | X-linked | | |
| RDX | Radixin | Hs.263671 | NM_002906 |
| RNASE4 | Angiogenin, ribonuclease, | Hs.283749 | NM_001145 |
| | RNase A family, 5 | | |
| SAA4 | Serum amyloid A4, | Hs.512677 | NM_006512 |
| | constitutive | | |
| SEPT8 | Septin 8 | Hs.533017 | AF440762 |
| SLC1A1 | Solute carrier family 1 | Hs.444915 | NM_004170 |
| | (neuronal/epithelial high | | |
| | affinity glutamate | | |
| | transporter, system Xag), | | |
| | member 1 | | |
| SLC4A1AP | Solute carrier family 4 (anion | Hs.306000 | NM_018158 |
| | exchanger), member 1, | | |
| | adaptor protein | | |
| SLC4A4 | Solute carrier family 4, | Hs.5462 | NM_003759 |
| | sodium bicarbonate | | |
| | cotransporter, member 4 | | |
| SLPI | Secretory leukocyte | Hs.517070 | NM_003064 |
| | protease inhibitor | | |
| | (antileukoproteinase) | | |
| ST13 | Suppression of | Hs.546303 | NM_003932 |
| | tumorigenicity 13 (colon | | |
| | carcinoma) (Hsp70 | | |
| | interacting protein) | | |
| STK32B | Serine/threonine kinase 32B | Hs.133062 | NM_018401 |
| SYTL1 | Synaptotagmin-like 1 | Hs.469175 | NM_032872 |
| TCEA3 | Transcription elongation | Hs.446354 | NM_003196 |
| | factor A (SII), 3 | | |
| TCEA3 | Transcription elongation | Hs.446354 | NM_003196 |
| | factor A (SII), 3 | | |
| TCEA3 | Transcription elongation | Hs.446354 | NM_003196 |
| | factor A (SII), 3 | | |
| TCN2 | Transcobalamin II; | Hs.417948 | NM_000355 |
| | macrocytic anemia | | |
| TIGA1 | TIGA1 | Hs.12082 | NM_053000 |
| TLR3 | Toll-like receptor 3 | Hs.29499 | NM_003265 |
| TPM4 | Tropomyosin 4 | Hs.466088 | NM_003290 |
| TTLL3 | Tubulin tyrosine ligase-like | Hs.517782 | NM_015644 |
| | family, member 3 | | |
| TUSC1 | Tumor suppressor candidate 1 | Hs.26268 | NM_001004125 |
| TUSC1 | Tumor suppressor candidate 1 | Hs.26268 | NM_001004125 |
| UNG2 | Uracil-DNA glycosylase 2 | Hs.3041 | NM_021147 |
| USP4 | Ubiquitin specific protease 4 | Hs.77500 | NM_003363 |
| | (proto-oncogene) | | |
| YME1L1 | YME1-like 1 (S. cerevisiae) | Hs.499145 | NM_139312 |
| ZADH1 | Zinc binding alcohol | Hs.98365 | NM_152444 |
| | dehydrogenase, domain | | |
| | containing 1 | | |
| ZNF292 | Zinc finger protein 292 | Hs.485892 | AK123473 |

*Searchable in the Unigene database available from the website of the National Center for Biotechnology Information of the United States National Institutes of Health, Bethesda, Maryland, United States of America.

TABLE 8
LAD Model - d1 Patterns

| | | Normalized |
|---|---|---|
| Subtype | Locus | Value |
| ccB | FAM44B < | −0.585 |
| ccA | FAM44B > | −0.585 |
| ccB | STK32B < | 0.42 |
| ccA | STK32B > | 0.42 |
| ccB | NETO2 < | −0.0985 |
| ccA | NETO2 > | −0.0985 |
| ccB | FBI4 < | 0.1035 |
| ccA | FBI4 > | 0.1035 |
| ccB | MAP7 < | 0.025 |
| ccA | MAP7 > | 0.025 |
| ccB | ST13 < | 0.1705 |
| ccA | ST13 > | 0.1705 |
| ccB | FBI4 < | 0.2165 |
| ccA | FBI4 > | 0.2165 |
| ccA | NCE2 < | 0.053 |
| ccB | NCE2 > | 0.053 |
| ccB | KIAA1648 < | 1.036 |
| ccA | KIAA1648 > | 1.036 |
| ccB | PURA < | 0.108 |
| ccA | PURA > | 0.108 |
| ccB | RAB3IP < | 0.1955 |
| ccA | RAB3IP > | 0.1955 |
| ccA | TPM4 < | −0.5045 |
| ccB | TPM4 > | −0.5045 |
| ccA | ALDH1A2 < | −1.4615 |
| ccB | ALDH1A2 > | −1.4615 |
| ccB | FZD1 < | 1.2345 |
| ccA | FZD1 > | 1.2345 |
| ccB | TCEA3 < | 2.3055 |
| ccA | TCEA3 > | 2.3055 |
| ccA | USP4 < | 0.6705 |
| ccB | USP4 > | 0.6705 |
| ccA | KCNK6 < | −0.735 |
| ccB | KCNK6 > | −0.735 |
| ccB | ACADL < | 0.9335 |
| ccA | ACADL > | 0.9335 |
| ccB | MAPT < | 2.82 |
| ccA | MAPT > | 2.82 |
| ccB | HOXC10 < | 0.5965 |
| ccA | HOXC10 > | 0.5965 |
| ccB | PTD012 < | 1.692 |
| ccA | PTD012 > | 1.692 |
| ccB | FLJ22104 < | −0.36 |
| ccA | FLJ22104 > | −0.36 |
| ccB | YME1L1 < | −0.1445 |
| ccA | YME1L1 > | −0.1445 |
| ccB | FLJ14249 < | 1.464 |
| ccA | FLJ14249 > | 1.464 |
| ccB | GIPC2 < | 0.2045 |
| ccA | GIPC2 > | 0.2045 |
| ccA | UNG2 < | −2.3535 |
| ccB | UNG2 > | −2.3535 |
| ccA | SLPI < | −2.385 |
| ccB | SLPI > | −2.385 |
| ccB | ACAA2 < | 1.1755 |
| ccA | ACAA2 > | 1.1755 |
| ccB | B3GNT6 < | 0.453 |
| ccA | B3GNT6 > | 0.453 |
| ccA | MGC40405 < | 1.0675 |
| ccB | MGC40405 > | 1.0675 |
| ccB | MAOB < | 3.0785 |
| ccA | MAOB > | 3.0785 |
| ccB | C9orf87 < | 0.056 |
| ccA | C9orf87 > | 0.056 |
| ccB | FLJ14054 < | 2.204 |
| ccA | FLJ14054 > | 2.204 |
| ccB | SLC4A1AP < | −0.011 |
| ccA | SLC4A1AP > | −0.011 |
| ccA | NPM3 < | −1.3135 |
| ccB | NPM3 > | −1.3135 |
| ccB | ACBD6 < | −0.3695 |
| ccA | ACBD6 > | −0.3695 |
| ccB | PRKAA2 < | 0.2845 |
| ccA | PRKAA2 > | 0.2845 |
| ccA | CDH3 < | −1.915 |
| ccB | CDH3 > | −1.915 |
| ccB | ZADH1 < | −0.1185 |
| ccA | ZADH1 > | −0.1185 |
| ccB | AQP11 < | −0.559 |
| ccA | AQP11 > | −0.559 |
| ccB | NUDT14 < | 0.091 |
| ccA | NUDT14 > | 0.091 |
| ccB | FLJ11200 < | 0.024 |
| ccA | FLJ11200 > | 0.024 |
| ccB | TCN2 < | 0.313 |
| ccA | TCN2 > | 0.313 |
| ccA | FLJ23867 < | −0.465 |
| ccB | FLJ23867 > | −0.465 |
| ccB | C13orf1 < | 0.7525 |
| ccA | C13orf1 > | 0.7525 |
| ccB | HSPA4L < | −0.5385 |
| ccA | HSPA4L > | −0.5385 |
| ccB | GIPC2 < | 0.3685 |
| ccA | GIPC2 > | 0.3685 |
| ccB | TCEA3 < | 1.579 |
| ccA | TCEA3 > | 1.579 |
| ccB | TCEA3 < | 1.283 |
| ccA | TCEA3 > | 1.283 |
| ccB | NPR3 < | 1.1865 |
| ccA | NPR3 > | 1.1865 |
| ccB | TLR3 < | 1.7685 |
| ccA | TLR3 > | 1.7685 |
| ccB | KIAA1043 < | 1.4045 |
| ccA | KIAA1043 > | 1.4045 |
| ccB | ARSE < | 2.1825 |
| ccA | ARSE > | 2.1825 |
| ccB | HOXA4 < | 1.696 |
| ccA | HOXA4 > | 1.696 |
| ccB | NPR3 < | 1.215 |
| ccA | NPR3 > | 1.215 |
| ccB | ACAT1 < | −0.398 |
| ccA | ACAT1 > | −0.398 |
| ccB | SLC1A1 < | 1.2895 |
| ccA | SLC1A1 > | 1.2895 |
| ccB | LEPROTL1 < | 1.132 |
| ccA | LEPROTL1 > | 1.132 |
| ccB | PMM1 < | 0.2675 |
| ccA | PMM1 > | 0.2675 |
| ccB | ITGA6 < | 0.659 |
| ccA | ITGA6 > | 0.659 |
| ccB | MAPT < | 0.9825 |
| ccA | MAPT > | 0.9825 |
| ccB | LOC57146 < | 0.0895 |
| ccA | LOC57146 > | 0.0895 |
| ccB | FLJ22104 < | −0.574 |
| ccA | FLJ22104 > | −0.574 |
| ccA | C5orf19 < | −1.6465 |
| ccB | C5orf19 > | −1.6465 |
| ccA | GALNT10 < | 1.107 |
| ccB | GALNT10 > | 1.107 |
| ccB | FLJ14249 < | 0.445 |
| ccA | FLJ14249 > | 0.445 |
| ccB | FLJ14146 < | 0.6405 |
| ccA | FLJ14146 > | 0.6405 |
| ccB | C11orf1 < | 0.721 |
| ccA | C11orf1 > | 0.721 |
| ccB | DNCH2 < | 0.3275 |
| ccA | DNCH2 > | 0.3275 |
| ccB | HIRIP5 < | 0.412 |
| ccA | HIRIP5 > | 0.412 |
| ccB | SEPT8 < | 0.9895 |
| ccA | SEPT8 > | 0.9895 |
| ccB | LOC134147 < | 0.6625 |
| ccA | LOC134147 > | 0.6625 |
| ccB | DSCR5 < | 0.2865 |
| ccA | DSCR5 > | 0.2865 |
| ccB | NMT2 < | −0.751 |
| ccA | NMT2 > | −0.751 |
| ccB | ADFP < | 2.505 |
| ccA | ADFP > | 2.505 |
| ccB | ALDH3A2 < | 0.456 |
| ccA | ALDH3A2 > | 0.456 |
| ccB | EHBP1 < | 0.3395 |
| ccA | EHBP1 > | 0.3395 |
| ccB | FAHD1 < | 0.502 |
| ccA | FAHD1 > | 0.502 |
| ccB | PHYH < | 0.17 |
| ccA | PHYH > | 0.17 |
| ccA | B3GALT7 < | −0.2365 |
| ccB | B3GALT7 > | −0.2365 |

With respect to Table 8, each entry includes the subtype, a locus, a normalized value, which corresponds to the expression level of the locus normalized as set forth hereinabove (see section entitled “Data Normalization”), whether the normalized value was greater than (>) or less than (<) the indicated amount in that subtype.

TABLE 9
LAD Model - d2 Patterns
ccA

| | | |
|---|---|---|
| B3GALT7 < | −0.2365 & MATN4 < | −0.0035 |
| B3GALT7 < | −0.2365 & GALNT10 < | 1.107 |
| B3GALT7 < | −0.2365 & CYB5R2 < | −0.2045 |
| B3GALT7 < | −0.2365 & MGC40405 < | 1.0675 |
| B3GALT7 < | −0.2365 & SAA4 < | −1.893 |
| B3GALT7 < | −0.2365 & TPM4 < | −0.5045 |
| B3GALT7 < | −0.2365 & ZNF292 < | 2.065 |
| B3GALT7 < | −0.2365 & NCE2 < | 0.053 |
| B3GALT7 < | −0.2365 & TTLL3 < | 0.935 |
| B3GALT7 < | −0.2365 & KCNK6 < | −0.735 |
| B3GALT7 < | −0.2365 & NPM3 < | −1.3135 |
| B3GALT7 < | −0.2365 & CDH3 < | −1.915 |
| B3GALT7 < | −0.2365 & C5orf19 < | −1.6465 |
| MATN4 < | −0.0035 & C5orf19 < | −1.6465 |
| MATN4 < | −0.0035 & SYTL1 < | −0.075 |
| MATN4 < | −0.0035 & NPM3 < | −1.3135 |
| MATN4 < | −0.0035 & KCNN4 < | 0.1215 |
| MATN4 < | −0.0035 & KCNK6 < | −0.735 |
| MATN4 < | −0.0035 & NCE2 < | 0.053 |
| MATN4 < | −0.0035 & IMP-2 < | 1.3365 |
| MATN4 < | −0.0035 & TPM4 < | −0.5045 |
| MATN4 < | −0.0035 & MGC40405 < | 1.0675 |
| MATN4 < | −0.0035 & GALNT10 < | 1.107 |
| LOC134147 > | 0.6625 | |
| DNCH2 > | 0.3275 | |
| C11orf1 > | 0.721 | |
| FLJ14146 > | 0.6405 | |
| AP4B1 < | 0.1395 & TPM4 < | −0.5045 |
| AP4B1 < | 0.1395 & C5orf19 < | −1.6465 |
| GALNT10 < | 1.107 & C5orf19 < | −1.6465 |
| GALNT10 < | 1.107 & SYTL1 < | −0.075 |
| GALNT10 < | 1.107 & CDH3 < | −1.915 |
| GALNT10 < | 1.107 & NPM3 < | −1.3135 |
| GALNT10 < | 1.107 & TTLL3 < | 0.935 |
| GALNT10 < | 1.107 & IMP-2 < | 1.3365 |
| GALNT10 < | 1.107 & ZNF292 < | 2.065 |
| GALNT10 < | 1.107 & TPM4 < | −0.5045 |
| GALNT10 < | 1.107 & CYB5R2 < | −0.2045 |
| C5orf19 < | −1.6465 & MGC40405 < | 1.0675 |
| C5orf19 < | −1.6465 & SAA4 < | −1.893 |
| C5orf19 < | −1.6465 & ZNF292 < | 2.065 |
| C5orf19 < | −1.6465 & NCE2 < | 0.053 |
| C5orf19 < | −1.6465 & TTLL3 < | 0.935 |
| C5orf19 < | −1.6465 & KCNK6 < | −0.735 |
| C5orf19 < | −1.6465 & KCNN4 < | 0.1215 |
| C5orf19 < | −1.6465 & NPM3 < | −1.3135 |
| C5orf19 < | −1.6465 & CDH3 < | −1.915 |
| FLJ22104 > | −0.574 | |
| LOC57146 > | 0.0895 | |
| SLC1A1 > | 1.2895 | |
| CYB5R2 < | −0.2045 & CDH3 < | −1.915 |
| CYB5R2 < | −0.2045 & KCNK6 < | −0.735 |
| CYB5R2 < | −0.2045 & NCE2 < | 0.053 |
| CYB5R2 < | −0.2045 & MGC40405 < | 1.0675 |
| NPR3 > | 1.215 | |
| TLR3 > | 1.7685 | |
| NPR3 > | 1.1865 | |
| TCEA3 > | 1.283 | |
| TCEA3 > | 1.579 | |
| GIPC2 > | 0.3685 | |
| FLJ23867 < | −0.465 | |
| TCN2 > | 0.313 | |
| NUDT14 > | 0.091 | |
| SYTL1 < | −0.075 & MGC40405 < | 1.0675 |
| SYTL1 < | −0.075 & SAA4 < | −1.893 |
| SYTL1 < | −0.075 & NCE2 < | 0.053 |
| SYTL1 < | −0.075 & KCNK6 < | −0.735 |
| SYTL1 < | −0.075 & CDH3 < | −1.915 |
| CDH3 < | −1.915 & MGC40405 < | 1.0675 |
| CDH3 < | −1.915 & SAA4 < | −1.893 |
| CDH3 < | −1.915 & TPM4 < | −0.5045 |
| CDH3 < | −1.915 & IMP-2 < | 1.3365 |
| CDH3 < | −1.915 & NCE2 < | 0.053 |
| CDH3 < | −1.915 & TTLL3 < | 0.935 |
| CDH3 < | −1.915 & KCNK6 < | −0.735 |
| CDH3 < | −1.915 & KCNN4 < | 0.1215 |
| CDH3 < | −1.915 & NPM3 < | −1.3135 |
| PRKAA2 > | 0.2845 | |
| NPM3 < | −1.3135 & MGC40405 < | 1.0675 |
| NPM3 < | −1.3135 & SAA4 < | −1.893 |
| NPM3 < | −1.3135 & TPM4 < | −0.5045 |
| NPM3 < | −1.3135 & NCE2 < | 0.053 |
| NPM3 < | −1.3135 & TTLL3 < | 0.935 |
| NPM3 < | −1.3135 & KCNK6 < | −0.735 |
| KCNN4 < | 0.1215 & TPM4 < | −0.5045 |
| MAOB > | 3.0785 | |
| MGC40405 < | 1.0675 & TTLL3 < | 0.935 |
| MGC40405 < | 1.0675 & IMP-2 < | 1.3365 |
| MGC40405 < | 1.0675 & ZNF292 < | 2.065 |
| MGC40405 < | 1.0675 & TPM4 < | −0.5045 |
| SAA4 < | −1.893 & IMP-2 < | 1.3365 |
| SAA4 < | −1.893 & ZNF292 < | 2.065 |
| SAA4 < | −1.893 & TPM4 < | −0.5045 |
| SLPI < | −2.385 | |
| UNG2 < | −2.3535 | |
| GIPC2 > | 0.2045 | |
| FLJ22104 > | −0.36 | |
| HOXC10 > | 0.5965 | |
| MAPT > | 2.82 | |
| ACADL > | 0.9335 | |
| KCNK6 < | −0.735 & TPM4 < | −0.5045 |
| KCNK6 < | −0.735 & ZNF292 < | 2.065 |
| KCNK6 < | −0.735 & IMP-2 < | 1.3365 |
| KCNK6 < | −0.735 & TTLL3 < | 0.935 |
| USP4 < | 0.6705 | |
| TCEA3 > | 2.3055 | |
| TTLL3 < | 0.935 & TPM4 < | −0.5045 |
| TTLL3 < | 0.935 & NCE2 < | 0.053 |
| FZD1 > | 1.2345 | |
| ALDH1A2 < | −1.4615 | |
| TPM4 < | −0.5045 & NCE2 < | 0.053 |
| TPM4 < | −0.5045 & ZNF292 < | 2.065 |
| ZNF292 < | 2.065 & NCE2 < | 0.053 |
| PURA > | 0.108 | |
| KIAA1648 > | 1.036 | |
| NCE2 < | 0.053 & IMP-2 < | 1.3365 |
| FBI4 > | 0.1035 | |
| STK32B > | 0.42 | |

ccB

| | | |
|---|---|---|
| FAHD1 < | 0.502 & PMM1 < | 0.2675 |
| FAHD1 < | 0.502 & ARSE < | 2.1825 |
| FAHD1 < | 0.502 & LOC90624 < | 0.411 |
| FAHD1 < | 0.502 & C13orf1 < | 0.7525 |
| FAHD1 < | 0.502 & FLJ11200 < | 0.024 |
| FAHD1 < | 0.502 & FLJ14054 < | 2.204 |
| FAHD1 < | 0.502 & B3GNT6 < | 0.453 |
| FAHD1 < | 0.502 & ESD < | −0.1545 |
| FAHD1 < | 0.502 & FLJ11588 < | 0.3335 |
| FAHD1 < | 0.502 & FLJ14249 < | 1.464 |
| FAHD1 < | 0.502 & FLT1 < | 3.161 |
| FAHD1 < | 0.502 & TUSC1 < | 1.184 |
| FAHD1 < | 0.502 & FLJ23834 < | 0.7815 |
| FAHD1 < | 0.502 & FAM44B < | −0.585 |
| FAHD1 < | 0.502 & NETO2 < | −0.0985 |
| FAHD1 < | 0.502 & MAP7 < | 0.025 |
| FAHD1 < | 0.502 & ST13 < | 0.1705 |
| FAHD1 < | 0.502 & FBI4 < | 0.2165 |
| FAHD1 < | 0.502 & KIAA0436 < | 0.036 |
| FAHD1 < | 0.502 & GHR < | 1.2595 |
| FAHD1 < | 0.502 & LOC119710 < | 0.516 |
| FAHD1 < | 0.502 & YME1L1 < | −0.1445 |
| FAHD1 < | 0.502 & RBMX < | 0.406 |
| FAHD1 < | 0.502 & MGC33887 < | 0.9085 |
| FAHD1 < | 0.502 & C9orf87 < | 0.056 |
| FAHD1 < | 0.502 & TIGA1 < | 1.9925 |
| FAHD1 < | 0.502 & SLC4A1AP < | −0.011 |
| FAHD1 < | 0.502 & ACBD6 < | −0.3695 |
| FAHD1 < | 0.502 & ZADH1 < | −0.1185 |
| FAHD1 < | 0.502 & RNASE4 < | 1.5125 |
| FAHD1 < | 0.502 & TUSC1 < | 1.643 |
| FAHD1 < | 0.502 & HSPA4L < | −0.5385 |
| FAHD1 < | 0.502 & KIAA1043 < | 1.4045 |
| FAHD1 < | 0.502 & HOXA4 < | 1.696 |
| FAHD1 < | 0.502 & KCNE3 < | 3.0215 |
| FAHD1 < | 0.502 & LEPROTL1 < | 1.132 |
| FAHD1 < | 0.502 & ITGA6 < | 0.659 |
| FAHD1 < | 0.502 & RDX < | −0.6795 |
| FAHD1 < | 0.502 & CWF19L2 < | −0.016 |
| FAHD1 < | 0.502 & SEPT8 < | 0.9895 |
| FAHD1 < | 0.502 & DSCR5 < | 0.2865 |
| FAHD1 < | 0.502 & BNIP3L < | 0.1595 |
| FAHD1 < | 0.502 & ALDH3A2 < | 0.456 |
| EHBP1 < | 0.3395 & ALDH3A2 < | 0.456 |
| EHBP1 < | 0.3395 & BNIP3L < | 0.1595 |
| EHBP1 < | 0.3395 & AFG3L2 < | −0.7015 |
| EHBP1 < | 0.3395 & DSCR5 < | 0.2865 |
| EHBP1 < | 0.3395 & RDX < | −0.6795 |
| EHBP1 < | 0.3395 & ITGA6 < | 0.659 |
| EHBP1 < | 0.3395 & LEPROTL1 < | 1.132 |
| EHBP1 < | 0.3395 & KCNE3 < | 3.0215 |
| EHBP1 < | 0.3395 & HOXA4 < | 1.696 |
| EHBP1 < | 0.3395 & KIAA1043 < | 1.4045 |
| EHBP1 < | 0.3395 & MGC32124 < | 1.1245 |
| EHBP1 < | 0.3395 & HSPA4L < | −0.5385 |
| EHBP1 < | 0.3395 & TUSC1 < | 1.643 |
| EHBP1 < | 0.3395 & RNASE4 < | 1.5125 |
| EHBP1 < | 0.3395 & ZADH1 < | −0.1185 |
| EHBP1 < | 0.3395 & ACBD6 < | −0.3695 |
| EHBP1 < | 0.3395 & TIGA1 < | 1.9925 |
| EHBP1 < | 0.3395 & C9orf87 < | 0.056 |
| EHBP1 < | 0.3395 & ACAA2 < | 1.1755 |
| EHBP1 < | 0.3395 & RBMX < | 0.406 |
| EHBP1 < | 0.3395 & DREV1 < | −0.6945 |
| EHBP1 < | 0.3395 & YME1L1 < | −0.1445 |
| EHBP1 < | 0.3395 & PTD012 < | 1.692 |
| EHBP1 < | 0.3395 & LOC119710 < | 0.516 |
| EHBP1 < | 0.3395 & ECHDC3 < | 0.7965 |
| EHBP1 < | 0.3395 & GHR < | 1.2595 |
| EHBP1 < | 0.3395 & KIAA0436 < | 0.036 |
| EHBP1 < | 0.3395 & FBI4 < | 0.2165 |
| EHBP1 < | 0.3395 & ST13 < | 0.1705 |
| EHBP1 < | 0.3395 & MAP7 < | 0.025 |
| EHBP1 < | 0.3395 & NETO2 < | −0.0985 |
| EHBP1 < | 0.3395 & FAM44B < | −0.585 |
| EHBP1 < | 0.3395 & SLC4A4 < | 2.618 |
| EHBP1 < | 0.3395 & FLJ23834 < | 0.7815 |
| EHBP1 < | 0.3395 & TUSC1 < | 1.184 |
| EHBP1 < | 0.3395 & RAB3IP < | 0.1955 |
| EHBP1 < | 0.3395 & FLT1 < | 3.161 |
| EHBP1 < | 0.3395 & FLJ14249 < | 1.464 |
| EHBP1 < | 0.3395 & C13orf1 < | −0.468 |
| EHBP1 < | 0.3395 & FLJ11588 < | 0.3335 |
| EHBP1 < | 0.3395 & ESD < | −0.1545 |
| EHBP1 < | 0.3395 & B3GNT6 < | 0.453 |
| EHBP1 < | 0.3395 & AQP11 < | −0.559 |
| EHBP1 < | 0.3395 & FLJ11200 < | 0.024 |
| EHBP1 < | 0.3395 & C13orf1 < | 0.7525 |
| EHBP1 < | 0.3395 & LOC90624 < | 0.411 |
| EHBP1 < | 0.3395 & ARSE < | 2.1825 |
| EHBP1 < | 0.3395 & ACAT1 < | −0.398 |
| EHBP1 < | 0.3395 & PMM1 < | 0.2675 |
| EHBP1 < | 0.3395 & MAPT < | 0.9825 |
| EHBP1 < | 0.3395 & FLJ14249 < | 0.445 |
| EHBP1 < | 0.3395 & HIRIP5 < | 0.412 |
| EHBP1 < | 0.3395 & ADFP < | 2.505 |
| EHBP1 < | 0.3395 & BAT4 < | 0.3685 |
| ALDH3A2 < | 0.456 & BAT4 < | 0.3685 |
| ALDH3A2 < | 0.456 & ADFP < | 2.505 |
| ALDH3A2 < | 0.456 & NMT2 < | −0.751 |
| ALDH3A2 < | 0.456 & HIRIP5 < | 0.412 |
| ALDH3A2 < | 0.456 & FLJ14249 < | 0.445 |
| ALDH3A2 < | 0.456 & MAPT < | 0.9825 |
| ALDH3A2 < | 0.456 & PMM1 < | 0.2675 |
| ALDH3A2 < | 0.456 & ACAT1 < | −0.398 |
| ALDH3A2 < | 0.456 & ARSE < | 2.1825 |
| ALDH3A2 < | 0.456 & LOC90624 < | 0.411 |
| ALDH3A2 < | 0.456 & C13orf1 < | 0.7525 |
| ALDH3A2 < | 0.456 & FLJ11200 < | 0.024 |
| ALDH3A2 < | 0.456 & AQP11 < | −0.559 |
| ALDH3A2 < | 0.456 & FLJ14054 < | 2.204 |
| ALDH3A2 < | 0.456 & B3GNT6 < | 0.453 |
| ALDH3A2 < | 0.456 & FLJ11588 < | 0.3335 |
| ALDH3A2 < | 0.456 & FLJ14249 < | 1.464 |
| ALDH3A2 < | 0.456 & RAB3IP < | 0.1955 |
| ALDH3A2 < | 0.456 & TUSC1 < | 1.184 |
| ALDH3A2 < | 0.456 & FLJ23834 < | 0.7815 |
| ALDH3A2 < | 0.456 & FAM44B < | −0.585 |

With respect to Table 9, the Table includes two halves: the top of relates to the ccA subtype and the bottom half relates to the ccB subtype. Each entry in the Table includes a locus, the expression level of which is compared (greater than (>) or less than (<) to a normalized value as was described hereinabove with respect to Table 8. In some instances, a locus is shown to be associated with a single subtype such as the entry in the top half of Table 9 that states that for ccA, the normalized value of the expression level of FLJ14146 is greater than 0.6405 (i.e., “FLJ14146>0.6405”). In other instances, a subtype is associated with the normalized values of more than one loci, such as the entry:

AP4B1<0.1395 & TPM4<−0.5045,

which indicates that ccA is associated with a normalized value for AP4B1 of less than 0.1395 and a normalized value of TPM4 of less than −0.5045.

TABLE 10
Training Set Non-Core Samples

| | Average LAD | Confidence | |
|---|---|---|---|
| Sample | score | Level | Prediction |
| 12 | −0.541798 | 1 | ccA |
| A6 | −0.418991 | 1 | ccA |
| E5 | −0.396952 | 1 | ccA |
| 1 | −0.177602 | 1 | ccA |
| E6 | −0.154244 | 1 | ccA |
| D6 | −0.146646 | 1 | ccA |
| 6 | −0.144308 | 1 | ccA |
| C3 | −0.054542 | 0.94 | ccA |
| 8 | 0.016424 | 0.63 | unclassified |
| 4 | 0.078228 | 0.98 | ccB |
| A16 | 0.361745 | 1 | ccB |
| E4 | 0.541945 | 1 | ccB |

To confirm that the genes identified by LAD are differentially expressed ccA and ccB ccRCC subtypes within individual tumors, primers for ccA overexpressed genes FLT1, FZD1, GIPC2, MAP7, and NPR3 were tested on available tumor samples using semi-quantitative RT-PCR. FIG. 4B demonstrates that each of these products can predict tumor classification for individual tumors. These results collectively indicate the potential for a particular gene set to correctly distinguish between the two ccRCC subtypes using RT-PCR, a platform immediately transferable to formalin-fixed, paraffin embedded tissues.

### Example 4

### Validation of ccRCC Subtypes

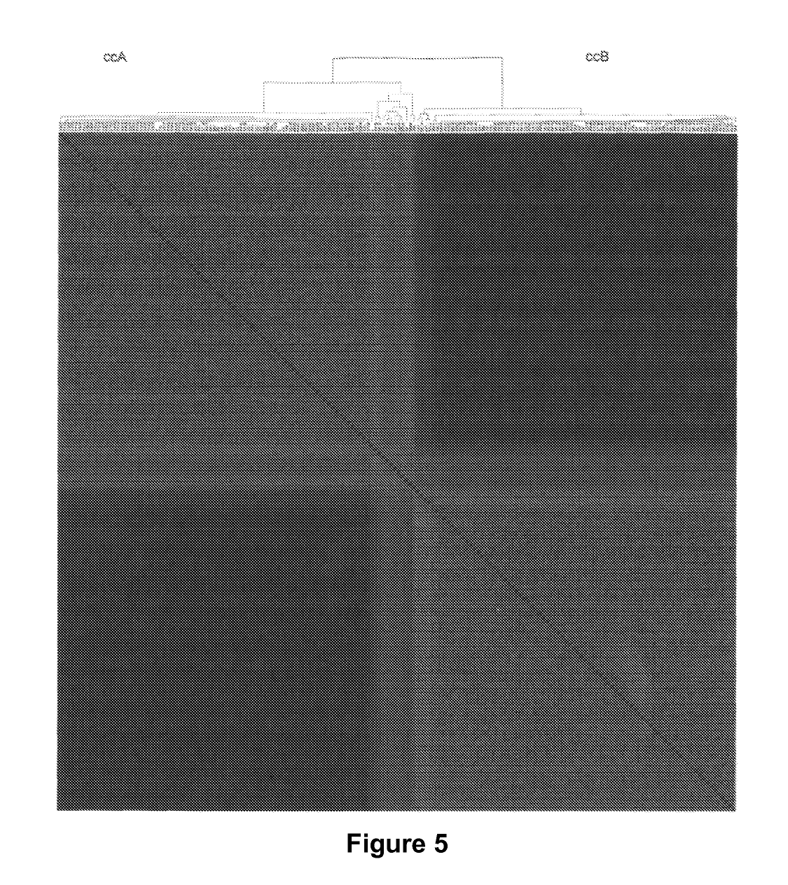

To validate the presence of two ccRCC subtypes in a second, independent dataset, ConsensusCluster (Seiler et al., 2010) and the LAD probe set were applied to 177 ccRCC microarrays generated using a different gene expression profiling technique (Zhao et al., 2006). FIG. 5 shows the same two strong clusters in the data, which remained stable when k was increased. The clusters were assigned to ccA or ccB by comparison of gene expression patterns to those in the primary dataset.

### Example 5

### Assignment of Individual Tumors

Assignment of tumors to a subtype with Cluster3.0 (traditional heatmaps) or ConsensusCluster required the presence of other tumors. Therefore, LAD score was employed to separately assign each individual tumor in the validation dataset to ccA or ccB, without assessing similarity to the rest of the tumors. Assignment was predicted for each sample 100 times with 80% pattern bootstrapping. A tumor was classified only if the assignment occurred in >75% of the prediction runs. Out of the 177 ccRCC tumors, 83 tumors were predicted to be ccA, 60 as ccB, and 34 remained unclassified with these stringent classification rules (see Table 11). When compared with the cluster assignment predicted by ConsensusCluster, a concordance of over 86% was identified, thus validating LAD predicted assignment as a sensitive measure of tumor assignment.

TABLE 11
Validation Set LAD Assignment

| | | Censoring | | | |
|---|---|---|---|---|---|
| | Survival | status | LAD | Confidence | Cluster |
| Sample | time | (DOD) | score | level | assignment |
| 9930 | 9 | 1 | 0.54963 | 1 | ccB |
| 9121 | 28 | 1 | 0.490051 | 1 | ccB |
| 109 | 9 | 1 | 0.483385 | 1 | ccB |
| 8710 | 6 | 1 | 0.425401 | 1 | ccB |
| 8807 | 55 | 1 | 0.424413 | 1 | ccB |
| 8822 | 0 | 0 | 0.421467 | 1 | ccB |
| 9003 | 12 | 1 | 0.420398 | 1 | ccB |
| 8726 | 251 | 0 | 0.413342 | 1 | ccB |
| 9818 | 11 | 1 | 0.357419 | 1 | ccB |
| 8820 | 10 | 1 | 0.355119 | 1 | ccB |
| 9871 | 21 | 1 | 0.353043 | 1 | ccB |
| 8607 | 24 | 1 | 0.331495 | 1 | ccB |
| 9411 | 59 | 1 | 0.320664 | 1 | ccB |
| 8603 | 4 | 1 | 0.300502 | 1 | ccB |
| 9122 | 3 | 1 | 0.298277 | 1 | ccB |
| 9105 | 56 | 0 | 0.294394 | 1 | ccB |
| 9006 | 1 | 1 | 0.292559 | 1 | ccB |
| 24 | 7 | 1 | 0.290463 | 1 | ccB |
| 9626 | 28 | 1 | 0.269176 | 1 | ccB |
| 9907 | 13 | 1 | 0.26237 | 1 | ccB |
| 9215 | 193 | 0 | 0.262358 | 1 | ccB |
| 8714 | 9 | 1 | 0.261657 | 1 | ccB |
| 8828 | 50 | 1 | 0.259963 | 1 | ccB |
| 244 | 23 | 1 | 0.236123 | 1 | ccB |
| 8914 | 4 | 1 | 0.232217 | 1 | ccB |
| 8918 | 7 | 1 | 0.230354 | 1 | ccB |
| 9603 | 6 | 0 | 0.229354 | 1 | ccB |
| 9611 | 152 | 0 | 0.228928 | 1 | ccB |
| 9101 | 206 | 1 | 0.2277 | 1 | ccB |
| 9406 | 94 | 1 | 0.227084 | 1 | ccB |
| 239 | 14 | 1 | 0.226257 | 1 | ccB |
| 9919 | 26 | 1 | 0.224839 | 1 | ccB |
| 16 | 41 | 1 | 0.200144 | 1 | ccB |
| 8917 | 32 | 1 | 0.199122 | 1 | ccB |
| 9721 | 131 | 0 | 0.169289 | 1 | ccB |
| 9210 | 2 | 1 | 0.167252 | 1 | ccB |
| 8922 | 1 | 1 | 0.163736 | 1 | ccB |
| 218 | 12 | 1 | 0.16123 | 1 | ccB |
| 9410 | 35 | 0 | 0.160754 | 0.99 | ccB |
| 8814 | 6 | 1 | 0.157191 | 1 | ccB |
| 312 | 32 | 1 | 0.156871 | 0.99 | ccB |
| 107 | 88 | 0 | 0.144776 | 0.99 | ccB |
| 9804 | 17 | 1 | 0.136229 | 0.96 | ccB |
| 101 | 93 | 0 | 0.134468 | 0.99 | ccB |
| 9306 | 15 | 1 | 0.134055 | 0.95 | ccB |
| 9021 | 172 | 1 | 0.132701 | 0.96 | ccB |
| 9812 | 38 | 1 | 0.131006 | 0.97 | ccB |
| 9409 | 97 | 1 | 0.120632 | 0.98 | ccB |
| 9214 | 132 | 0 | 0.109876 | 0.94 | ccB |
| 8931 | 0 | 0 | 0.108221 | 0.91 | ccB |
| 9934 | 11 | 1 | 0.104735 | 0.92 | ccB |
| 9103 | 10 | 1 | 0.102875 | 0.94 | ccB |
| 301 | 25 | 1 | 0.101217 | 0.85 | ccB |
| 9799 | 131 | 0 | 0.099651 | 0.91 | ccB |
| 9711 | 8 | 1 | 0.074373 | 0.81 | ccB |
| 9817 | 54 | 0 | 0.070522 | 0.81 | ccB |
| 9308 | 1 | 1 | 0.067514 | 0.8 | ccB |
| 9933 | 107 | 0 | 0.066754 | 0.82 | ccB |
| 8722 | 255 | 0 | 0.060713 | 0.79 | ccB |
| 8605 | 18 | 1 | 0.060119 | 0.79 | ccB |
| 9515 | 156 | 0 | 0.041393 | 0.67 | unclassified |
| 245 | 70 | 0 | 0.03966 | 0.65 | unclassified |
| 235 | 2 | 1 | 0.039177 | 0.67 | unclassified |
| 9401 | 38 | 1 | 0.039037 | 0.62 | unclassified |
| 8906 | 238 | 0 | 0.038116 | 0.64 | unclassified |
| 9022 | 13 | 1 | 0.038053 | 0.65 | unclassified |
| 9616 | 42 | 0 | 0.037552 | 0.7 | unclassified |
| 208 | 77 | 0 | 0.036995 | 0.65 | unclassified |
| 9725 | 136 | 0 | 0.035563 | 0.59 | unclassified |
| 9915 | 23 | 1 | 0.034846 | 0.71 | unclassified |
| 13 | 39 | 1 | 0.03031 | 0.58 | unclassified |
| 8709 | 23 | 1 | 0.027212 | 0.58 | unclassified |
| 29 | 6 | 1 | 0.011612 | 0.54 | unclassified |
| 9211 | 6 | 0 | 0.01006 | 0.52 | unclassified |
| 9935 | 5 | 1 | 0.010007 | 0.51 | unclassified |
| 8913 | 236 | 0 | 0.008257 | 0.5 | unclassified |
| 9511 | 27 | 1 | 0.006308 | 0.44 | unclassified |
| 8708 | 3 | 1 | 0.005965 | 0.45 | unclassified |
| 9001 | 227 | 0 | 0.005279 | 0.44 | unclassified |
| 9123 | 205 | 0 | 0.004458 | 0.45 | unclassified |
| 8915 | 19 | 1 | −0.00028 | 0.44 | unclassified |
| 9013 | 163 | 1 | −0.00046 | 0.49 | unclassified |
| 9007 | 22 | 1 | −0.00172 | 0.45 | unclassified |
| 9722 | 131 | 0 | −0.02491 | 0.49 | unclassified |
| 9119 | 181 | 0 | −0.02535 | 0.57 | unclassified |
| 9407 | 2 | 0 | −0.02921 | 0.63 | unclassified |
| 8818 | 34 | 1 | −0.02929 | 0.63 | unclassified |
| 19 | 35 | 1 | −0.02952 | 0.67 | unclassified |
| 28 | 39 | 1 | −0.02961 | 0.59 | unclassified |
| 9921 | 16 | 0 | −0.03221 | 0.63 | unclassified |
| 9615 | 29 | 1 | −0.03222 | 0.6 | unclassified |
| 9908 | 18 | 0 | −0.05203 | 0.74 | unclassified |
| 9820 | 105 | 0 | −0.05494 | 0.74 | unclassified |
| 4 | 94 | 0 | −0.0565 | 0.75 | ccA |
| 111 | 37 | 0 | −0.05676 | 0.75 | ccA |
| 9895 | 39 | 1 | −0.05857 | 0.83 | ccA |
| 9610 | 2 | 1 | −0.05862 | 0.77 | ccA |
| 9008 | 223 | 0 | −0.0589 | 0.77 | ccA |
| 8704 | 170 | 1 | −0.05986 | 0.7 | unclassified |
| 9124 | 4 | 1 | −0.08224 | 0.86 | ccA |
| 9014 | 29 | 1 | −0.08586 | 0.89 | ccA |
| 8621 | 106 | 0 | −0.08994 | 0.9 | ccA |
| 104 | 91 | 0 | −0.08996 | 0.92 | ccA |
| 217 | 76 | 0 | −0.09114 | 0.88 | ccA |
| 9502 | 8 | 1 | −0.09138 | 0.9 | ccA |
| 9624 | 119 | 0 | −0.09145 | 0.9 | ccA |
| 201 | 10 | 1 | −0.09386 | 0.85 | ccA |
| 8927 | 60 | 1 | −0.11768 | 0.96 | ccA |
| 99 | 99 | 0 | −0.1209 | 0.95 | ccA |
| 8606 | 271 | 0 | −0.12304 | 0.94 | ccA |
| 8811 | 38 | 1 | −0.12358 | 0.96 | ccA |
| 8910 | 16 | 1 | −0.12404 | 0.97 | ccA |
| 9608 | 38 | 0 | −0.12499 | 0.98 | ccA |
| 9011 | 40 | 1 | −0.12555 | 0.97 | ccA |
| 223 | 75 | 0 | −0.14499 | 0.99 | ccA |
| 8809 | 14 | 1 | −0.15109 | 1 | ccA |
| 114 | 76 | 1 | −0.15746 | 1 | ccA |
| 11 | 6 | 1 | −0.1585 | 0.99 | ccA |
| 9203 | 193 | 0 | −0.1585 | 0.99 | ccA |
| 9312 | 38 | 1 | −0.17782 | 1 | ccA |
| 9209 | 41 | 1 | −0.18636 | 1 | ccA |
| 9827 | 118 | 0 | −0.18695 | 1 | ccA |
| 9605 | 84 | 0 | −0.18739 | 1 | ccA |
| 1 | 104 | 0 | −0.18989 | 1 | ccA |
| 9918 | 48 | 1 | −0.18992 | 1 | ccA |
| 9925 | 110 | 0 | −0.19011 | 1 | ccA |
| 9726 | 42 | 1 | −0.19347 | 0.99 | ccA |
| 9928 | 110 | 0 | −0.19522 | 1 | ccA |
| 9112 | 1 | 1 | −0.21474 | 1 | ccA |
| 9102 | 4 | 1 | −0.21563 | 1 | ccA |
| 209 | 77 | 0 | −0.21798 | 1 | ccA |
| 9707 | 138 | 0 | −0.22049 | 1 | ccA |
| 9910 | 59 | 0 | −0.2208 | 1 | ccA |
| 9931 | 58 | 1 | −0.24829 | 1 | ccA |
| 9712 | 135 | 0 | −0.24837 | 1 | ccA |
| 112 | 79 | 1 | −0.24863 | 1 | ccA |
| 9212 | 194 | 0 | −0.24926 | 1 | ccA |
| 9408 | 169 | 0 | −0.24963 | 1 | ccA |
| 204 | 79 | 0 | −0.25154 | 1 | ccA |
| 18 | 97 | 1 | −0.25278 | 1 | ccA |
| 9307 | 81 | 0 | −0.25528 | 1 | ccA |
| 9507 | 90 | 0 | −0.27597 | 1 | ccA |
| 9932 | 108 | 0 | −0.2764 | 1 | ccA |
| 8802 | 37 | 1 | −0.2802 | 1 | ccA |
| 9503 | 45 | 1 | −0.28277 | 1 | ccA |
| 9514 | 150 | 1 | −0.28517 | 1 | ccA |
| 9510 | 103 | 1 | −0.30721 | 1 | ccA |
| 26 | 25 | 0 | −0.31172 | 1 | ccA |
| 9316 | 4 | 1 | −0.31334 | 1 | ccA |
| 108 | 44 | 1 | −0.31352 | 1 | ccA |
| 9020 | 21 | 1 | −0.31463 | 1 | ccA |
| 9614 | 39 | 1 | −0.34556 | 1 | ccA |
| 8902 | 38 | 1 | −0.34594 | 1 | ccA |
| 25 | 96 | 0 | −0.34627 | 1 | ccA |
| 8517 | 14 | 0 | −0.34673 | 1 | ccA |
| 238 | 71 | 0 | −0.36856 | 1 | ccA |
| 8816 | 201 | 0 | −0.3734 | 1 | ccA |
| 229 | 15 | 1 | −0.37519 | 1 | ccA |
| 8817 | 57 | 0 | −0.37637 | 1 | ccA |
| 8925 | 223 | 1 | −0.37838 | 1 | ccA |
| 9109 | 170 | 0 | −0.4066 | 1 | ccA |
| 9811 | 125 | 0 | −0.41189 | 1 | ccA |
| 9010 | 40 | 1 | −0.43433 | 1 | ccA |
| 9923 | 110 | 0 | −0.43554 | 1 | ccA |
| 306 | 34 | 1 | −0.44222 | 1 | ccA |
| 10 | 19 | 1 | −0.46575 | 1 | ccA |
| 310 | 66 | 0 | −0.4691 | 1 | ccA |
| 9118 | 15 | 0 | −0.47093 | 1 | ccA |
| 9903 | 14 | 0 | −0.47145 | 1 | ccA |
| 9710 | 18 | 0 | −0.47231 | 1 | ccA |
| 9405 | 9 | 1 | −0.49997 | 1 | ccA |
| 9922 | 15 | 1 | −0.50275 | 1 | ccA |
| 103 | 22 | 1 | −0.53045 | 1 | ccA |
| 6 | 103 | 0 | −0.5313 | 1 | ccA |
| 9402 | 73 | 0 | −0.56812 | 1 | ccA |
| 207 | 6 | 1 | −0.58801 | 1 | ccA |
| 9815 | 122 | 0 | −0.62547 | 1 | ccA |

### Example 6

### VHL Pathway Analysis

With the ability to assign individual tumors to ccA or ccB, it was possible to further investigate an intriguing aspect of the pathway analysis disclosed herein. Several of the pathways overexpressed in ccA tumors are typically considered as being perturbed in ccRCC (i.e., angiogenesis is considered a defining feature of ccRCC). A number of genes (e.g., EPAS1, EGLN3, PDGFC, HIG2, and CA9) tightly correlated with aspects of VHL inactivation and hypoxia inducible factor (HIF) signaling were found to be overexpressed in ccA relative to ccB.

LAD analysis was applied to a previously generated dataset (Gordon et al., 2008) that was well annotated for VHL inactivation. Out of the 21 tumors, 10 were predicted to be ccA, 6 as ccB, and 5 as unclassified (Table 12). In each category, there were VHL wild type tumors, HIF1 and HIF2 overexpressing tumors and HIF2 only overexpressing tumors. An analysis of VHL status also demonstrated the presence of VHL mutations and/or methylation in both the ccA and ccB clusters (Table 1).

TABLE 12
Assignment of Arrays from Gordan et al., 2008

| | | Average | Confidence | |
|---|---|---|---|---|
| HIF status | Sample | LAD score | score | Subtype |
| H1H2 | TB3806 | −0.355684 | 1 | ccA |
| H1H2 | TB3852 | −0.272272 | 1 | ccA |
| H1H2 | TB3820 | −0.228271 | 1 | ccA |
| H1H2 | TB3823 | −0.104881 | 0.97 | ccA |
| H1H2 | TB3901 | −0.103752 | 1 | ccA |
| H2 | TB3812 | −0.226498 | 1 | ccA |
| H2 | TB4084 | −0.22551 | 1 | ccA |
| H2 | TB3821 | −0.062398 | 0.9 | ccA |
| WT | TB3895 | −0.141951 | 1 | ccA |
| WT | TB4037 | −0.068126 | 0.96 | ccA |
| H1H2 | TB3825 | 0.143501 | 0.99 | ccB |
| H1H2 | 3860 | 0.203623 | 1 | ccB |
| H2 | TB3809 | 0.064538 | 0.92 | ccB |
| H2 | TB3816 | 0.100317 | 0.98 | ccB |
| WT | GOGO256 | 0.247851 | 1 | ccB |
| WT | TB3874 | 0.30537 | 1 | ccB |
| H1H2 | TB3844 | −0.022302 | 0.69 | uncl |
| H2 | TB3826 | −0.021929 | 0.71 | uncl |
| H2 | TB3940 | −0.021229 | 0.66 | uncl |
| H2 | TB3822 | 0.017215 | 0.63 | uncl |
| WT | 4045 | 0.037016 | 0.73 | uncl |

Assignment was predicted for each sample 100 times with 80% pattern bootstrapping. A tumor was classified only if the assignment occurred in >75% of the prediction runs.

These data suggested that ccA and ccB, despite having a similar frequency of VHL inactivation, were characterized by activation of different dominant biologic pathways, resulting in distinct patterns of gene expression.

### Example 7

### ccA and ccB have Different Survival Outcomes

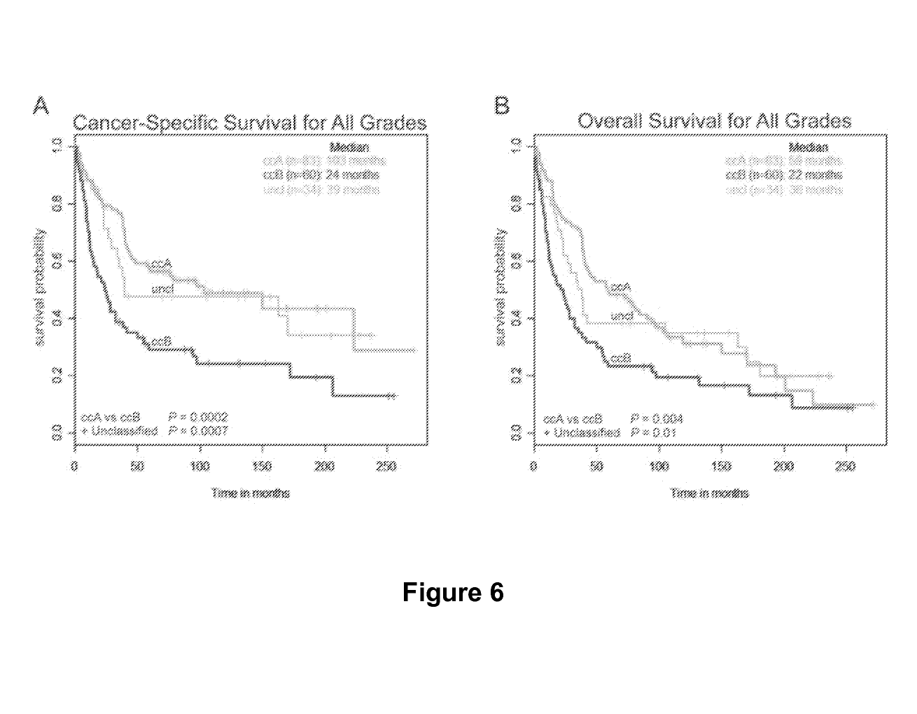

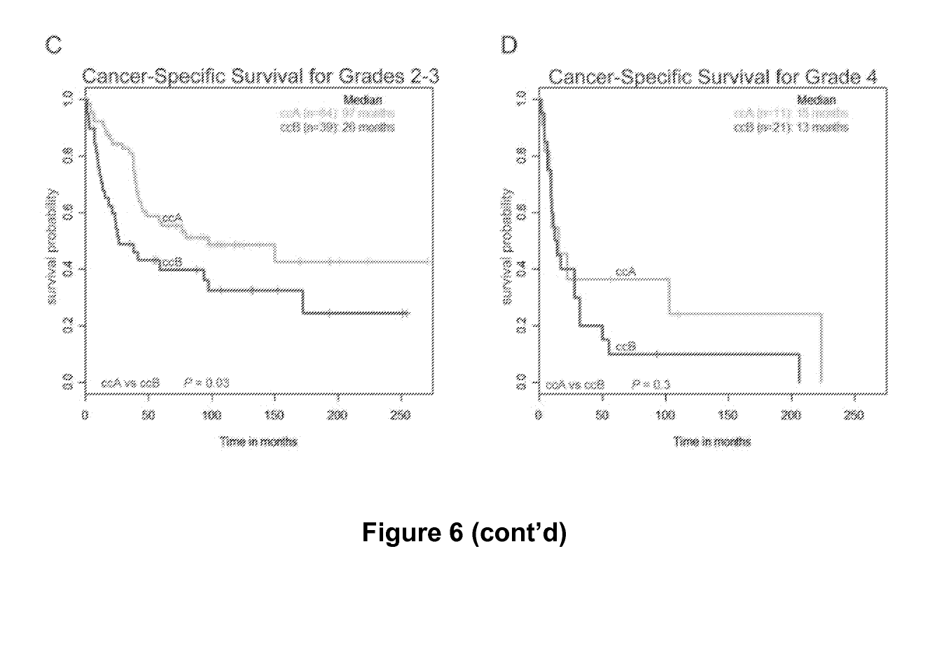

Given that VHL is inactivated in tumors of both subtypes, whether the underlying differences in tumor biology showed survival differences was determined. Cancer specific survival and overall survival for the ccA and ccB classes from the 177 tumor validation set were plotted using Kaplan-Meier curves (FIG. 6A-6B), calculating 95% confidence intervals (Table 13). For cancer specific survival (FIG. 6A), the ccA subtype was associated with a highly significant survival advantage over ccB patients (p=0.0002, median survival of 8.6 vs. 2 years). At five years, cancer specific survival was 56% in ccA patients and only 29% in ccB patients. FIG. 6B shows the same trend for overall survival, with a significantly greater survival for ccA patients over ccB patients (p=0.004, median survival of 4.9 vs. 1.8 years). At five years, survival for ccA patients is 48%, while only 23% for ccB patients.

TABLE 13
Survival Times with 95% Confidence Intervals

| | | | | |
|---|---|---|---|---|
| | Median | 95% CI for | 5 Year | 95% CI for 5 |
| | survival | median survival | Survival | year survival |
| Subtype | (years) | (years) | (%) | (%) |

DSS Survival Analysis

| | | | | |
|---|---|---|---|---|
| ccA | 8.6 | 3.8-N/A | 56 | 45-67 |
| ccB | 2.0 | 1.0-3.2 | 29 | 18-41 |

OS Survival Analysis

| | | | | |
|---|---|---|---|---|
| ccA | 4.9 | 3.3-7.8 | 48 | 37-58 |
| ccB | 1.8 | 0.9-2.6 | 23 | 14-35 |

Calculated median and 5 year survival times with 95% confidence intervals (CI) for ccA and ccB subtypes in disease specific (DSS) and overall survival (OS) analysis.

### Example 8

### ccA/ccB Subtype Associates with Clinical Variables

Fuhrman grade, tumor size (T stage), and performance status, the covariates in the UCLA International Staging System (UISS) for predicting outcome in newly diagnosed patients (Zisman et al., 2001), were evaluated and compared with the molecular classifications disclosed herein with regard to survival outcomes. Molecular classification strongly associated with tumor stage (p=0.009) and grade (p=0.0007), but not performance status (p=0.5684). 78% of grade 1 and 69% of stage 1 tumors clustered as ccA, while and 65% of grade 4 and 58% of stage 4 tumors cluster as ccB tumors. This result was consistent with the observation that low grade ccRCC tumors tend to have better prognosis, and high grade tumors tend toward poor prognosis (Frank et al., 2002). This observation also suggests that the biological characteristics responsible for grade and stage-specific prognosis in ccRCC are encompassed in the classification schema. FIG. 6C demonstrates that the ccA/ccB subtype still significantly correlates with survival when limiting analysis to intermediate grade (grade 2-3) tumors. A Kaplan-Meier curve limited to the highly aggressive grade 4 tumors shows a convergence of subtype-specific survival (FIG. 6D).

### Example 9

### Molecular Classification is Independently Associated with Survival

To determine how classification schema disclosed herein compared with current standard clinical parameters as a prognostic factor, univariate Cox regression analyses were performed (Table 14). Molecular subtype is strongly associated with survival, with an HR of 2.2 (p=0.0003). Even in the absence of stage 4 (metastatic) tumors, subtype has a strong association with survival (HR=2.143, p=0.0233). Additionally, the use of Schwartz Bayesian Criterion (SBC; Kass & Raftery, 1995) suggests that whether the tumor is classified by ccA/ccB/unclassified, ccA/ccB, or LAD score, the measures are strongly associated with survival, with difference in adjusted SBC values of 8, 8.3, and 9 respectively. These results suggest that defining a tumor as ccA or ccB may be an important prognostic indicator for predicting outcome from patients with ccRCC.

TABLE 14
Univariable Cox Regression Analysis
for Disease Specific Survival**

| Covariate of Interest | HR | 95% CI | p-value |
|---|---|---|---|
| Subtype ccA/ccB | 2.2 | 1.4-3.4 | 0.0003 |
| Subtype all ccA/ccB | 1.8 | 1.2-2.7 | 0.0033 |
| Subtype ccA/ccB/uncl | 1.5 | 1.2-1.9 | 0.0004 |
| LAD score | 1.2 | 1.1-1.3 | 0.0002 |
| Grade | 1.9 | 1.4-2.5 | <0.0001 |
| Stage | 3.4 | 2.6-4.3 | <0.0001 |
| Performance Status | 1.7 | 1.4-2.1 | <0.0001 |

**Hazard ratios, with 95% confidence intervals (CI) and p-values, were calculated for the predicted subtype (ccA vs. ccB), LAD score, stage, grade and performance status (PS). Analysis of “Subtype ccA/ccB” used only the 143 tumors classified using bootstrap analysis. Analysis of “Subtype all ccA/ccB” included all 177 tumors classified by LAD score without using the 75% confidence cutoff. Analysis of “Subtype ccA/ccB/uncl” included all 177 tumors classified as ccA, ccB, or unclassified by LAD score and bootstrapping. The HR for LAD score is per 0.1 units.

Multivariate analyses were then performed to determine whether the classification schema disclosed herein was still independently associated with survival outcomes in the context of stage, grade, and performance status. The dichotomous classification of ccA/ccB provides a significant association with survival at the 0.1 level (p=0.089), likely influenced by the smaller sample size of the 143 classified tumors. Increasing sample size to 177 by including unclassified tumors, the trichotomous classification increased significance to p=0.0736. Statistical analyses often show that continuous variables provide more statistical discrimination. In fact, LAD score is an independent predictor of survival (p=0.0027) and is more predictive of outcome than Fuhrman grade (p=0.0308). These data intimate that the classification schema presented in this paper may provide independent prognostic information over and above that provided by standard clinical parameters.

### Discussion of the Examples

Clear cell renal cell carcinoma (ccRCC) is the predominant RCC subtype, but even within this classification, the natural history is heterogeneous and difficult to predict. A sophisticated understanding of the molecular features most discriminatory for the underlying tumor heterogeneity is desirably predicated on identifiable and biologically meaningful patterns of gene expression.

As disclosed herein, gene expression microarray data were analyzed using software that implements iterative unsupervised consensus clustering algorithms, to identify the optimal molecular subclasses, without clinical or other classifying information. ConsensusCluster analysis identified two distinct subtypes of ccRCC within the training set, designated clear cell type A (ccA) and B (ccB). Based on the core tumors, or most well-defined arrays, in each subtype, Logical Analysis of Data (LAD) defined a minimum highly predictive gene set that could then be used to classify additional tumors individually. The subclasses were corroborated in a validation dataset of 177 tumors and analyzed for clinical outcome. Based on individual tumor assignment, tumors designated ccA have markedly improved disease-specific survival compared to ccB (median survival of 8.6 vs. 2.0 years; p=0.002). Analyzed by both univariate and multivariate analysis, the classification schema independently associated with survival. Using patterns of gene expression based on a defined gene set, ccRCC was classified into two robust subclasses based on inherent molecular features that ultimately correspond to marked differences in clinical outcome. This classification schema thus provides a molecular stratification applicable to individual tumors that has implications to influence treatment decisions, define biological mechanisms involved in ccRCC tumor progression, and direct future drug discovery.

Thus, unsupervised consensus clustering algorithms can identify distinct classifications of histologically similar tumors based on machine learning algorithms. In this analysis, a small gene set distinguished two inherent molecular subtypes of ccRCC (ccA and ccB), characterized by divergent biological pathways and a highly significant association with survival outcomes. This analysis provides a representative method to discriminate molecular subgroups of tumors that can be informative of tumor biology or influence tumor behavior.

A fundamental problem in gene expression analysis of human tumors is the measurement of genetic noise in pairwise comparisons across thousands of independent and dependent variables. The combined use of PCA, consensus clustering, and LAD disclosed herein was robust, and, more importantly, identified stable clusters within patterns of gene expression. This method was highly reproducible and able to classify samples into molecular and clinically meaningful categories. Within these categories, “Core clusters” are sets of non-overlapping samples that are distinguishable from each other with high accuracy. This representative embodiment of the presently disclosed methods of tumor analysis permitted a refined assignment into gene expression-defined classifications and yielded predictive gene signatures based on a manageable sized number of gene features. These properties allowed for the identification of limited sets of highly predictive molecular features (i.e., genes) useful for the classification of individual samples outside of the primary analysis.

The extension of biomarker molecular profiles to small groups of genes, which can assign classification to individual tumors, is a major step forward toward the development of a clinically relevant biomarker. Ultimately, such a classification scheme can be applied with such measures as quantitative RT-PCR.

Disclosed herein is the discovery that there are likely only two primary subtypes of ccRCC stable under bootstrap analysis, although further subclassifications within these subtypes might be identified in much larger datasets, and rare tumors might represent unusual variants. Using the LAD predictions in the validation set, a third group of tumors shared pattern features with both ccA and ccB tumors. Such a third group, or other suggested classifications, might represent an intermediate manifestation of tumors undergoing progression from ccA to the ccB subtype, or which simply share common characteristics of both groups.

The subtypes ccA and ccB were associated with a significant difference in survival outcome, with ccA patients having a markedly better prognosis. The continuous variable of LAD score proved to be an independent predictor of survival.

Pathway analysis showed that the better prognosis ccA group relatively overexpressed genes associated with hypoxia, angiogenesis, fatty acid metabolism, and organic acid metabolism, whereas ccB tumors overexpressed a more aggressive panel of genes that regulate EMT, the cell cycle, and wound healing. Intriguingly, ccA overexpressed genes associated with components of hypoxia and angiogenesis pathways, processes known to be broadly dysregulated in clear cell RCC. VHL inactivation and subsequent activation of the hypoxia response pathway is so highly correlated with ccRCC that many of these pathways are expected to be upregulated in virtually all ccRCC tumors. As expected, using both training set tumors and LAD assigned gene expression arrays from Gordan et al., 2008, VHL inactivation was identified in both clusters. Thus, ccB might have acquired additional genetic events which supplement VHL pathway events, contributing to a more biologically immature and aggressive phenotype that overwhelms the signature associated with VHL inactivation.

Finally, the robust panel of genes disclosed herein, the expression levels of which can be employed to classify individual tumor samples into ccA and ccB subtypes with high accuracy, can provide a valuable resource for clinical decisions for patients following nephrectomy regarding frequency of surveillance or choices for adjuvant therapy. This panel can thus provide the basis for assigning subtypes of ccRCC to individual tumor specimens.

### REFERENCES

The references listed below as well as all references cited in the specification including, but not limited to patents, patent application publications, journal articles, and database entries (including but not limited to GENBANK® and/or Ensembl database entries, also including all annotations and references cited therein) are incorporated herein by reference to the extent that they supplement, explain, provide a background for, or teach methodology, techniques, and/or compositions employed herein.
Albert et al. (1992) J Virol 66:5627-5630.
Alexay et al. (1996) The International Society of Optical Engineering 2705/63.
Alexe et al. (2006) Cancer Informatics 2:243-274.
Alexe et al. (2007) Cancer Res 67:10669-10676.
American Cancer Society, Inc. (2009) “Cancer facts and figures. 2009”. Atlanta, Ga., United States of America.
Ausubel et al. (2002) Short Protocols in Molecular Biology, Fifth ed. Wiley, New York, N.Y., United States of America.
Ausubel et al. (2003) Current Protocols in Molecular Biology, John Wiley & Sons, Inc., New York, N.Y., United States of America.
Banks et al. (2006) Cancer Res. 66:2000-2011.
Bej et al. (1991) Appl Environ Microbiol 57:3529-3534.
Boom et al. (1990) J Clin Microbiol 28:495-503.
Brown & Botstein (1999) Nature Genet 21:33-37.
Buffone et al. (1991) Clin Chem 37:1945-1949.
Busch et al. (1992) Transfusion 32:420-425.
Cha & Thilly (1993) PCR Methods Appl 3:S18-S29.
Chiodi et al. (1992) J Clin Microbiol 30:255-258.
Crama et al. (1988) Annals of Operation Research 16:299-326.
Dalgin et al. (2007) BMC Bioinformatics 8:291.
De Francesco (1998) The Scientist 12:16.
de Hoon et al. (2004) Bioinformatics 20:1453-1454.
de Waard et al. (1999) Gene 226:1-8.
DeRisi et al. (1996) Nat Genet 14:457-460.
Dubiley et al. (1997) Nuc Acids Res 25:2259-2265.
Eberwine (1996) Biotechniques 20:584-591.
Englert (2000) in Schena, ed., Microarray Biochip Technology, pp. 231-246, Eaton Publishing, Natick, Mass., United States of America.
Espejo et al. (2002) Biochem J 367:697-702.
Everitt & Dunn G (2001) Applied Multivariate Data Analysis. London: Hodder Arnold Publication.
Fang et al. (2002) Chembiochem 3:987-991.
Fodor et al. (1991) Science 251:767-773.
Fodor et al. (1993) Nature 364:555-556.
Frank et al. (2002) J Urol 168:2395-2400.
Furge et al. (2004) Cancer Res 64:4117-4121.
Gordan et al. (2008) Cancer Cell 14:435-446.
Granjeuad et al. (1999) BioEssays 21:781-790.
Guedon et al. (2000) Anal Chem 72(24):6003-6009.
Haab et al. (2001) Genome Biol 2.
Hamel et al. (1995) J Clin Microbiol 33:287-291.
Hammer & Bonates (2006) Annals of Operation Research 148:203-225.
Heaton et al. (2001) Proc Natl Acad Sci USA 98(7):3701-3704.
Herman et al. (1994) Proc Natl Acad Sci USA 91:9700-9704.
Herrewegh et al. (1995) J Clin Microbiol 33:684-689.
Houseman et al. (2002) Nat Biotechnol 20:270-274.
Huang et al. (2009) Nat Protoc 4:44-57.
Hubank & Schatz (1994) Nuc Acids Res 22:5640-5648.
Innis et al. (eds) (1990) PCR Protocols: A Guide to Methods and Applications, Academic Press, San Diego, Calif., United States of America.
Ivanova et al. (1995) Nuc Acids Res 23:2954-2958.
Izraeli et al. (1991) Nuc Acids Res 19:6051.
Jolliffe (2002) Principal Component Analysis (2^{nd} Edition). New York: Springer-Verlag. 487 p.
Kass & Raftery (1995) JASA 90:773-795.
Kato (1995) Nuc Acids Res 23:3685-3690.
Kohonen (2001) Self-Organizing Maps. New York: Springer.
Kohsaka & Carson (1994) J Clin Lab Anal 8:425-455.
Kriegler (1990) Gene Transfer and Expression: A Laboratory Manual, Stockton Press, New York, N.Y., United States of America.
Liang & Pardee (1992) Science 257:967-971.
Lam et al. (2005) J Urol 174:466-472.
Lanciotti et al. (1992) J Clin Microbiol 30:545-551.
Linz et al. (1990) J Clin Chem Clin Biochem 28:5-13.
Lipshutz et al. (1999) Nat Genet 21:20-24.
Liu & Hlady (1996) Coll Sur B 8:25-37.
Lockhart & Winzeler (2000) Nature 405:827-836.
Lockhart et al. (1996) 14 Nat Biotechnol 1675-1680.
MacBeath & Schreiber (2000) Science 289:1760-1763.
McCaustland et al. (1991) J Virol Methods 35:331-342.
McGall et al. (1996) 93 Proc Nat Acad Sci USA 13555-13460.
McPherson et al. (1995) PCR 2: A Practical Approach, IRL Press, New York, N.Y., United States of America.
Millar et al. (1995) Anal Biochem 226:325-330.
Monti et al. (2003) Machine Learning Journal 52:91-118.
Mootha et al. (2003) Nat Genet 34:267-273.
Natarajan et al. (1994) PCR Methods Appl 3:346-350.
Nelson et al. (2001) Anal Chem 73(1):1-7.
Nickerson et al. (2008) Clin Cancer Res 14:4726-4734.
Nogueira & Kim (2008) Urol Oncol 26:113-124.
O'Donnell et al. (1997) Anal Chem 69:2438-2443.
Paik et al. (2004) N Engl J Med 351:2817-2826.
Paladichuk (1999) The Scientist 13(16):20-23.
PCT International Patent Application Publications WO 93/09668; 95/11755; WO 97/14028; WO 99/19515; WO 99/32660; WO 99/63385; WO 01/13120; WO 01/14589; WO 01/23082; WO 2004/046098; WO 2004/110244; WO 2006/089268; WO 2007/001324; WO 2007/056332; WO 2007/070252.
Perou et al. (2000) Nature 406:747-752.
Perucho et al. (1995) Methods Enzymol 254:275-290.
Piétu et al. (1996) Genome Res 6:492-503.
Randolph & Waggoner (1995) Nuc Acids Res 25:2923-2929.
Ratner & Castner (1997) in Vickerman, ed., Surface Analysis: The Principal Techniques, John Wiley & Sons, New York, United States of America.
Reddy et al. (2008) BMC Med Inform Decis Mak 8:30.
Robertson & Walsh-Weller (1998) Methods Mol Biol 98:121-154.
Rose (2000) in Schena, ed., Microarray Biochip Technology, pp. 19-38, Eaton Publishing, Natick, Mass., United States of America.
Roux (1995) PCR Methods Appl 4:S185-S194.
Rupp et al. (1988) Bio Techniques 6:56-60.
Salisbury et al. (2002) J Am Chem Soc 124:14868-14870.
Sambrook & Russell (2001) Molecular Cloning: A Laboratory Manual, Cold Spring Harbor Press, Cold Spring Harbor, N.Y.
Sapolsky & Lipshutz (1996) Genomics 33:445-456.
Schena et al. (1995) Science 270:467-470.
Schena et al. (1996) Proc Natl Acad Sci USA 93:10614-10619.
Seiler et al. (2010) ConsensusCluster: a stand-alone software tool for unsupervised cluster discovery in numerical data. OMICS14:109-113.
Seong (2002) Clin Diagn Lab Immunol 9:927-930.
Shalon et al. (1996) Genome Res 6:639-645.
Shimkets et al. (1999) Nature Biotechnology 17:798-803.
Shoemaker et al. (1996) Nat Genet 14:450-456.
Shriver-Lake (1998) in Cass & Ligler, eds., Immobilized Biomolecules in Analysis, pp. 1-14, Oxford Press, Oxford, United Kingdom.
Silhavy et al. (1984) Experiments with Gene Fusions, Cold Spring Harbor Laboratory, Cold Spring Harbor, N.Y., United States of America.
Skubitz et al. (2006) J Lab Clin Med 147:250-267.
Smith (1998) The Scientist 12(14):21-24.
Sorbellini et al. (2005) J Urol 173:48-51.
Sorlie et al. (2001) Proc Natl Acad Sci USA 98:10869-10874.
Southern (1975) J Mol Biol 98:503-517.
Stolle et al. (1998) Hum Mutat 12:417-423.
Strain & Chmielewski (2001) Bio Techniques 30(6):1286-1291.
Subramanian et al. (2005) Proc Nat Acad Sci USA 102:15545-15550.
Takahashi et al. (2001) Proc Natl Acad Sci USA 98:9754-9759.
Tanaka et al. (1994) J Gen Virol 75:2691-2698.
Telenius et al. (1992) Genomics 13:718-725.
Tijssen (ed.) (1993) Laboratory Techniques in Biochemistry and Molecular Biology: Hybridization With Nucleic Acid Probes, Part I Theory and Nucleic Acid Preparation, Elsevier Press, New York, N.Y., United States of America.
Tusher et al. (2001) Proc Natl Acad Sci USA 98:5116-5121.
U.S. Patent Application Publication Nos. 20020009767; 20020155495; 20030049701; 20040033625; 20040219575; 20050255491; 20060275851; 20070099254; 20080260763; 20090062194.
U.S. Pat. Nos. 4,683,195; 4,683,202; 4,729,947; 5,143,854; 5,207,880; 5,230,781; 5,346,603; 5,360,523; 5,534,125; 5,571,388; 5,743,960; 5,800,992; 5,837,832; 5,843,767; 5,846,717; 5,871,697; 5,871,918; 5,916,524; 5,965,352; 5,968,745; 5,974,164; 5,985,557; 5,994,069; 6,001,567; 6,017,696; 6,066,457; 6,086,737; 6,090,543; 6,123,819; 6,127,127; 6,162,603; 6,185,561; 6,225,059; 6,229,911; 6,245,508.
van de Vijver et al. (2002) N Engl J Med 347:1999-2009.
Vankerckhoven et al. (1994) J Clin Microbiol 30:750-753.
Velculescu et al. (1995) Science 270:484-487.
Velculescu et al. (1997) Cell 88:243-251.
Wall et al. (2003) In: Berrar et al. (eds.) A Practical Approach to Microarray Data Analysis. Boston, Mass.: Kluwer Academic Publishers. pp. 91-109.
Wang et al. (1998) Proc Natl Acad Sci USA 86:9717-9721.
Warrington et al. (2000) in Schena, ed., Microarray Biochip Technology, pp. 119-148, Eaton Publishing, Natick, Mass., United States of America.
Williams (1989) Bio Techniques 7:762-769.
Williams et al. (1990) Nuc Acids Res 18(22):6531-6535.
Worley et al. (2000) in Schena, ed., Microarray Biochip Technology, pp. 65-86, Eaton Publishing, Natick, Mass., United States of America.
Yang et al. (1998) Science 282:2244-2246.
Yershov et al. (1996) Proc Natl Acad Sci USA 93:4319-4918.
Young et al. (2008) Adv Anat Pathol 15:28-38.
Zhao et al. (2006) PLoS Med 3:e13.
Zhu et al. (2001) Science 293:2101-2105.
Zisman et al. (2001) J Clin Oncol 19:1649-1657.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for generating a prognostic signature for a subject with clear cell renal cell carcinoma (ccRCC), the method comprising determining expression levels for three or more genes listed in Table 7 in ccRCC cells obtained from the subject, wherein the determining provides a prognostic signature for the subject.

2. The method of claim 1, comprising determining expression levels for at least 4, 5, 6, 7, 8 9, 10, or all 120 of the genes listed in Table 7 in ccRCC cells obtained from the subject.

3. The method of claim 1, comprising determining expression levels for each of FLT1, FZD1, GIPC2, MAP7, and NPR3 in ccRCC cells obtained from the subject.

4. The method of claim 1, further comprising comparing the prognostic signature determined to a standard.

5. The method of claim 4, wherein the standard comprises a gene expression profile of the one or more genes obtained from ccA cells obtained from one or more subjects with ccRCC, an expression profile of the one or more genes obtained from ccB cells obtained from one or more subjects with ccRCC, or both.

6. The method of claim 4, wherein the comparing comprises employing a Single Sample Predictor (SSP), Principal Component Analysis (PCA), consensus clustering, logical analysis of data (LAD) analyses, or a combination thereof.

7. The method of claim 6, wherein the gene expression profile of the one or more genes obtained from ccA cells in the standard comprises a mean expression level for the one or more genes in the ccA cells, the expression profile of the one or more genes obtained from ccB cells, or both.

8. The method of claim 7, wherein if the standard comprises both gene expression profiles, the mean expression levels are determined separately for the one or more genes in the ccA cells and the one or more genes in the ccB cells

9. The method of claim 7, wherein the standard comprises both gene expression profiles and the method further comprises assigning with the SSP, PCA, consensus clustering, and/or LAD analyses the prognostic signature to either the mean expression level for the three or more genes in the ccA cells or the mean expression level for the three or more genes in the ccB cells.

10. The method of claim 9, wherein the assigning comprises employing a Spearman correlation.

11. The method of one of claim 9, wherein the assigning step is performed by a suitably-programmed computer.

12. The method of claim 1, wherein the subject is a human.

13. A method for assessing risk of an adverse outcome of a subject with clear cell renal cell carcinoma (ccRCC), the method comprising:
determining a mean expression level for three or more genes selected from among those genes listed in Table 7 in a biological sample comprising ccRCC cells obtained from subject; and
comparing the expression levels determined to a standard.

14. The method of claim 13, wherein the three or more genes are selected from among FLT1, FZD1, GIPC2, MAP7, and NPR3.

15. The method of claim 13, wherein the subject is a human.

16. The method of claim 13, wherein evidence of the expression level is obtained by a method comprising gene expression profiling.

17. The method of claim 15, wherein the gene expression profiling method is a PCR-based method, a microarray based method, or an antibody-based method.

18. The method of claim 16, wherein the expression levels are normalized relative to the expression levels of one or more reference genes.

19. The method of claim 13, comprising determining the expression levels of at least four of the genes listed in Table 7.

20. The method of claim 19, comprising determining the expression levels of at least five of the genes listed in Table 7.

21. The method of claim 13, wherein the comparing comprises employing a Single Sample Predictor (SSP), Principal Component Analysis (PCA), consensus clustering, logical analysis of data (LAD) analyses, or a combination thereof.

22. The method of claim 21, wherein the gene expression profile of the one or more genes obtained from ccA cells in the standard comprises a mean expression level for the one or more genes in the ccA cells, the expression profile of the one or more genes obtained from ccB cells, or both.

23. The method of claim 22, wherein if the standard comprises both gene expression profiles, the mean expression levels are determined separately for the one or more genes in the ccA cells and the one or more genes in the ccB cells

24. The method of claim 22, wherein the standard comprises both gene expression profiles and the method further comprises assigning with the SSP, PCA, consensus clustering, and/or LAD analyses the prognostic signature to either the mean expression level for the three or more genes in the ccA cells or the mean expression level for the three or more genes in the ccB cells.

25. The method of claim 24, wherein the assigning comprises employing a Spearman correlation.

26. The method of one of claim 24, wherein the assigning step is performed by a suitably-programmed computer.

27. A method for predicting a clinical outcome of a treatment in a subject having clear cell renal cell carcinoma (ccRCC), the method comprising:
(a) determining the expression levels of three or more genes listed in Table 7, optionally three or more of FLT1, FZD1, GIPC2, MAP7, and NPR3, in a biological sample comprising ccRCC cells obtained from the ccRCC of the subject; and
(b) comparing the expression levels determined to a standard, wherein the comparing is predictive of the clinical outcome of the treatment in the subject.

28. The method of claim 27, wherein the clinical outcome is expressed in terms of Recurrence-Free Interval (RFI), Overall Survival (OS), Disease-Free Survival (DFS), or Distant Recurrence-Free Interval (DRFI).

29. The method of claim 27, comprising determining the expression levels of at least four, at least five, or at least ten of the genes listed in Table 7.

30. The method of claim 27, where the treatment is selected from among surgical resection, chemotherapy, molecular targeted therapy, immunotherapy, and combinations thereof.

31. The method of claim 27, wherein the comparing comprises employing a Single Sample Predictor (SSP), Principal Component Analysis (PCA), consensus clustering, logical analysis of data (LAD) analyses, or a combination thereof.

32. The method of claim 27, wherein the standard comprises a gene expression profile of the one or more genes obtained from ccA cells obtained from one or more subjects with ccA, an expression profile of the one or more genes obtained from ccB cells obtained from one or more subjects with ccB, or both.

33. The method of claim 32, wherein the gene expression profile of the one or more genes obtained from ccA cells in the standard comprises a mean expression level for the one or more genes in the ccA cells, the expression profile of the one or more genes obtained from ccB cells, or both.

34. The method of claim 33, wherein if the standard comprises both gene expression profiles, the mean expression levels are determined separately for the one or more genes in the ccA cells and the one or more genes in the ccB cells

35. The method of claim 33, wherein the standard comprises both gene expression profiles and the method further comprises assigning with the SSP, PCA, consensus clustering, and/or LAD analyses the prognostic signature to either the mean expression level for the three or more genes in the ccA cells or the mean expression level for the three or more genes in the ccB cells.

36. The method of claim 35, wherein the assigning comprises employing a Spearman correlation.

37. The method of one of claims 31 and 35, wherein the comparing step, the assigning step, or both is/are performed by a suitably-programmed computer.

38. The method of claim 32, wherein the gene expression profile of the three or more genes obtained from ccA cells in the standard comprises a mean expression level for the three or more genes in the ccA cells, the expression profile of the three or more genes obtained from ccB cells, or both, and optionally further wherein if the standard comprises both gene expression profiles, the mean expression levels are determined separately for the three or more genes in the ccA cells and the three or more genes in the ccB cells.

39. The method of claim 27, wherein the subject is a human.

40. An array comprising polynucleotides that hybridize specifically to at least three genes listed in Table 7 or comprising specific peptide or polypeptide gene products of at least three genes listed in Table 7.

41. The array of claim 40, wherein each specific peptide or polypeptide gene product present on the array is present thereon in an amount, relative to each other specific peptide or polypeptide gene product that is present on the array, that is reflective of the expression level of its corresponding gene in clear cell renal cell carcinoma (ccRCC) cells obtained from a subject with ccRCC.

42. The array of claim 40, wherein the specific peptide or polypeptide gene products are present on the array such that the array is interrogatable with at least one antibody that specifically binds to one of the specific peptide or polypeptide gene products.

43. The array of claim 40, wherein the array comprises at least one polynucleotide or specific peptide or polypeptide gene product for each of FLT1, FZD1, GIPC2, MAP7, and NPR3.


SEQUENCE LISTING (20 sequences)

SEQ ID NO: 1 - DNA, 6499 bp, Homo sapiens
  CDS (286)..(2349)
atcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacgagaggacggactctggcggccgggtcgttggccgcggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaaaggactcattaaaaagtaacagttgtctcatatcatcttgatttattgtcactgttgctaactttcaggctcggaggagatgctcctcccaaaatgagttcggagatgatagcagtaataatgagacccccgggccccagctctgggccccccattcaggccgagggggctgctccggggggccgacttggtgcacgtttggatttggaggatccctgcactgccttctctgtgtttgttgctcttgctgttttctcctgcctgataaacaacaacttgggatgatcctttccttccattttgatgccaacctctttttatttttaagtgttgaagctgcacaaactgaataatttaaacaaatgctggtttctgccaaagatggacacgaataagttaattttccagctcagaatgagtacagttgaatttgagactctgtcggacttctgcctggttttatttgggactatttcatctgctcttgatttgtaaatagcacctggatagcaagttataatgcttatttatttgaaaatgctttttttttttttacgttaagcacatttatcttgaactggagcttctaaaatgggccccaggggtgcaagatgttggtgtaattcagagatagtaaaggtttatcgcagtgtgaattataagagtccatccaaatcaacgtcccctccctcctctcatgcgatccaggtaattatgcagttagtgccacagtagactagcctagcaaagggtttgctccttgctgtctctgactgcaccacacagctattgatggcagctgaaagaaagtggatcatgccttaattttaaatattcctgtcctctggttattattttaaggaacttcatcatgttaaaatgacagcattcaaaggtgtaccacaatcaatttatcaaggaaataaaggctattgtaaccagagatttaatgcattcttctaaatgtaaatttaaaatttgccctttaaaaaagtccactttccccatatgcaaatgttaataggatttttatggggattaagaagcggcaaaactacagaagcagaattcaaagtaatttaaaaaatacacaccagttttaaatcaagagaagttgtaatctcttgttttaagcttgcgtttgagggaaaatgactttttcaccaatttaatatgcattgttctgttgtttttatttatgattgatcattatatgtgacttgcataaactatttaaaaaaaaaaactataatgaccaaaatagccatggctgagaaacacagtggctgggcagttcaataggaggtgacaatatgacaacttctcaagcttgggaactcaccagactgtttcctcctttaggtaacagattctgtcccacggctaaacttgtctttcacgtgggaattgcttttgtcaaacgtgaaagagtaaacaatagcatttccccagaatgccagttttatggagccccaaatgctctgaaaacaattagtaacctggaagttgtcagcccaaaggaaagaaaaatcaattgtatcttgaaattttacctatggctctttggcctggcttctttgttcattataagttagtgtgttccttcaggaaacaatgccttaataccatagaacatgggggccttaatagttgctaacattaaaaaagcaaacagaatgattgagggatccttatgaaaacaaaatggtgaattggacatgcagaacctaccatttccttcccctgtttgcaatttttgtggggaggggaggatgttagtatttacaaaagatgattttaagaacttccaagagatgagtttaagaattccatagagtattagttgttcactgtgtaattaatccttccggagagtctttttttttttttttaaagaaacttttgggtgggttttgttttttattagttaccctaggggtatgttaccctggggtatgaagggaggtgaagataacggaggggggagaaaaaaaaaaggagaaaaaaggagcctaaaatggggaataattgaaatggaacagggggtgtgaggctggttcctcagtccccattccaaacggaggatagaagctgtgtatttatgtgacctggcagatctctggggccataacactgaaaagtgaaagaacctggtgggcagctatctttggctactgataaccagcagaaatgtctgttaattctgattttctcaatttgaagggatcagctacactgttaaattttggaaagccactacctacttccatcaagtaacttaggtttcgaaatatgggttcaacgcacctcccttattcaaaatgtcaaaatagattattataatgtataaagtaagaattgacaaaatatgattcttgggttgattggtcatttagaaactagccaaaagtgagacttttaatgtagaacatttttcagaaatgggtacaaagaaaaatgcatattactgtatatttcagagtgtttatgtgaaccttgtatttaattgagagtcccatgtacgttctgcagcctttttgctgcttctatcatctgaagtttgtgtagtacaaataaggcctttgggattcttaatgacatttatgttaaaatgttctcttctctttaaacaccgttttccaatccacctgtcagggagtccaaatcgtgtctgtgttgatgatgctatactttgtagctagaaaaacaattttagtgttgtgggctctgtattcagacttcctttttacaagaccgatgggcagtgatagattattttatcatatttaatgcatgggaaatagtgtgctgaggaagctattaaaagtataactcagtgaattgggtctgagttttaaatgagatatttcaaaattggcttgccactgtaaaagcgactaaataataatatgatactgttctttatgatcttgtcatgtttcactgatatgtttggggtcttcactatgtaaaaaatgtcaaaattgtaatgagcaagcatgtacaagtagtcgtaaatcaaaggttttaaacaggactgcattttcaattaggaaaagctgtttggcagatagcatccaatgcaaaaacagaaatatcgtaacgttctgcttagtgggcaagataagataggaaagacatgctcaaagaggcaaaagaatcattgctatcattcattctacactagtttgaagaagtttttgtacatcagagcacttccttcagcacacttttttgccttcagatttcattttttataaaatgagaagactaatgataaactgtagaaatcaaaatttattgagaaatctgtttctcctaacagatagtaaccctgccatgatatactacttcaacaatgttataaaatttatgtgataatatacattttaacctgggatttctaaattgctttaacaaatgctaatcctgagagttgccctgcaggactcaaaagggaaaggttttgggacgtggcagaaccctgcagggacatggaattaaggccattgcaatgtatcatctttgtagcattgtcatcactcctaagctgccttcacagttttagtacactaagatgaggaaatcgaaaatgggcagagaaagctcatactgtataattgaagacagtgacagagaacgtgtcagttatgccaaaactcttttgatttctgttccaggatttccaacaagaggggaaaggaatgacttgggagggtgggaaagacattaggagttgtttttattttttaccttggaagctttagctaccaatccagtaccctcctaactagaatgtatacacatcagcaggactgactgactacttcattagagatatactgtactcattgggggccttgggggtactgctgttcttatgtgggattttaatgttgtaatgtattgcatcttaatgtattgaattcattttgttgtactatattggttggcattttattaaaataaattgtattgtatcatatttgtatgttttaagagaaaataatataaaatacaatatttgtactattatatagtgcaaaaactacaaatctgtgcctctgcctcttgaattaattctttggttgcttgcatttgggaagggaatggagaaaggaaagaaccaataaagctttcaaagttcaag

SEQ ID NO: 2 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 3 - DNA, 4350 bp, Homo sapiens
  CDS (414)..(2357)
agttgagggattgacacaaatggtcaggcggcggcggcggagaaggaggcggaggcgcaggggggagccgagcccgctgggctgcggagagttgcgctctctacggggccgcggccactagcgcggcgccgccagccgggagccagcgagccgagggccaggaaggcgggacacgaccccggcgcgccctagccacccgggttctccccgccgcccgcgcttcatgaatcgcaagtttccgcggcggcggcggctgcggtacgcagaacaggagccgggggagcgggccgaaagcggcttgggctcgacggagggcacccgcgcagaggtctccctggccgcagggggagccgccgccggccgtgcccctggcagccccagcggagcggcgccaagagaggagccgagaaagtatggctgaggaggaggcgcctaagaagtcccgggccgccggcggtggcgcgagctgggaactttgtgccggggcgctctcggcccggctggcggaggagggcagcggggacgccggtggccgccgccgcccgccagttgacccccggcgattggcgcgccagctgctgctgctgctttggctgctggaggctccgctgctgctgggggtccgggcccaggcggcgggccaggggccaggccaggggcccgggccggggcagcaaccgccgccgccgcctcagcagcaacagagcgggcagcagtacaacggcgagcggggcatctccgtcccggaccacggctattgccagcccatctccatcccgctgtgcacggacatcgcgtacaaccagaccatcatgcccaacctgctgggccacacgaaccaggaggacgcgggcctggaggtgcaccagttctaccctctagtgaaagtgcagtgttccgctgagctcaagttcttcctgtgctccatgtacgcgcccgtgtgcaccgtgctagagcaggcgctgccgccctgccgctccctgtgcgagcgcgcgcgccagggctgcgaggcgctcatgaacaagttcggcttccagtggccagacacgctcaagtgtgagaagttcccggtgcacggcgccggcgagctgtgcgtgggccagaacacgtccgacaagggcaccccgacgccctcgctgcttccagagttctggaccagcaaccctcagcacggcggcggagggcaccgtggcggcttcccggggggcgccggcgcgtcggagcgaggcaagttctcctgcccgcgcgccctcaaggtgccctcctacctcaactaccacttcctgggggagaaggactgcggcgcaccttgtgagccgaccaaggtgtatgggctcatgtacttcgggcccgaggagctgcgcttctcgcgcacctggattggcatttggtcagtgctgtgctgcgcctccacgctcttcacggtgcttacgtacctggtggacatgcggcgcttcagctacccggagcggcccatcatcttcttgtccggctgttacacggccgtggccgtggcctacatcgccggcttcctcctggaagaccgagtggtgtgtaatgacaagttcgccgaggacggggcacgcactgtggcgcagggcaccaagaaggagggctgcaccatcctcttcatgatgctctacttcttcagcatggccagctccatctggtgggtgatcctgtcgctcacctggttcctggcggctggcatgaagtggggccacgaggccatcgaagccaactcacagtattttcacctggccgcctgggctgtgccggccatcaagaccatcaccatcctggcgctgggccaggtggacggcgatgtgctgagcggagtgtgcttcgtggggcttaacaacgtggacgcgctgcgtggcttcgtgctggcgcccctcttcgtgtacctgtttatcggcacgtcctttctgctggccggctttgtgtcgctcttccgcatccgcaccatcatgaagcacgatggcaccaagaccgagaagctggagaagctcatggtgcgcattggcgtcttcagcgtgctgtacactgtgccagccaccatcgtcatcgcctgctacttctacgagcaggccttccgggaccagtgggaacgcagctgggtggcccagagctgcaagagctacgctatcccctgccctcacctccaggcgggcggaggcgccccgccgcacccgcccatgagcccggacttcacggtcttcatgattaagtaccttatgacgctgatcgtgggcatcacgtcgggcttctggatctggtccggcaagaccctcaactcctggaggaagttctacacgaggctcaccaacagcaaacaaggggagactacagtctgagacccggggctcagcccatgcccaggcctcggccggggcgcagcgatcccccaaagccagcgccgtggagttcgtgccaatcctgacatctcgaggtttcctcactagacaactctctttcgcaggctcctttgaacaactcagctcctgcaaaagcttccgtccctgaggcaaaaggacacgagggcccgactgccagagggaggatggacagacctcttgccctcacactctggtaccaggactgttcgcttttatgattgtaaatagcctgtgtaagatttttgtaagtatatttgtatttaaatgacgaccgatcacgcgtttttctttttcaaaagtttttaattatttagggcggtttaaccatttgaggcttttccttcttgcccttttcggagtattgcaaaggagctaaaactggtgtgcaaccgcacagcgctcctggtcgtcctcgcgcgcctctccctaccacgggtgctcgggacggctgggcgccagctccggggcgagttcagcactgcggggtgcgactagggctgcgctgccagggtcacttcccgcctcctccttttgccccctccccctccttctgtcccctccctttctttcctggcttgaggtaggggctcttaaggtacagaactccacaaaccttccaaatctggaggagggcccccatacattacaattcctcccttgctcggcggtggattgcgaaggcccgtcccttcgacttcctgaagctggatttttaactgtccagaactttcctccaacttcatgggggcccacgggtgtgggcgctggcagtctcagcctccctccacggtcaccttcaacgcccagacactcccttctcccaccttagttggttacagggtgagtgagataaccaatgccaaactttttgaagtctaatttttgaggggtgagctcatttcattctctagtgtctaaaacctggtatgggtttggccagcgtcatggaaagatgtggttactgagatttgggaagaagcatgaagctttgtgtgggttggaagagactgaagatatgggttataaaatgttaattctaattgcatacggatgcctggcaaccttgcctttgagaatgagacagcctgcgcttagattttaccggtctgtaaaatggaaatgttgaggtcacctggaaagctttgttaaggagttgatgtttgctttccttaacaagacagcaaaacgtaaacagaaattgaaaacttgaaggatatttcagtgtcatggacttcctcaaaatgaagtgctattttcttatttttaatcaaataactagacatatatcagaaactttaaaatgtaaaagttgtacactttcaacattttattacgattattattcagcagcacattctgaggggggaacaattcacaccaccaataataacctggtaagatttcaggaggtaaagaaggtggaataattgacggggagatagcgcctgaaataaacaaaatatgggcatgcatgctaaagggaaaatgtgtgcaggtctactgcattaaatcctgtgtgctcctcttttggatttacagaaatgtgtcaaatgtaaatctttcaaagccatttaaaaatattcactttagttctctgtgaagaagaggagaaaagcaatcctcctgattgtattgttttaaactttaagaatttatcaaaatgccggtacttaggacctaaatttatctatgtctgtcatacgctaaaatgatattggtctttgaatttggtatacatttattctgttcactatcacaaaatcatctatatttatagaggaatagaagtttatatatatataataccatatttttaatttcacaaataaaaaattcaaagttttgtacaaaattatatggattttgtgcctgaaaataatagagcttgagctgtctgaactattttacattttatggtgtctcatagccaatcccacagtgtaaaaattca

SEQ ID NO: 4 - protein, 647 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQAGGGAPPHPPMSPDFTVFMIKYLMTLIVGITSGFWIWSGKTLNSWRKFYTRLTNSKQGETTV

SEQ ID NO: 5 - DNA, 2838 bp, Homo sapiens
  CDS (191)..(1138)
gggtgggcgggcggctgctccggtccaggggtggaggtcggggccctgacccgacgcagccaggcggaagcgcggctgccattggaggctgcttttacctgcgcggggcccggggcgcaaagtccgaggcgccggggggaggaggcggcggacggcagcgcaggtgggcccgcgctctcggccctgcaagatgcccctgaagctgcgggggaagaagaaggccaagtccaaggagaccgccgggctggtggagggcgagccgacgggcgcgggcggcgggagcctctcagcgtcccgggctcccgcacgcaggctggtcttccacgcgcagctggcgcacggtagtgccacgggccgagtggagggcttctccagcatccaggagctctacgcccagatcgcgggcgcgtttgaaatctcgccgtcggagatcttatattgcactttaaacacacctaaaattgacatggaaagactcttaggaggacaactaggactagaagatttcatatttgcccatgtgaaaggaatcgaaaaagaagtgaatgtgtataaatctgaggattcacttggtctcaccattacagataatggtgttggctatgcttttataaagagaattaaagatggtggtgttattgactcagttaaaacaatctgtgttggggatcatattgaatccataaatggagaaaatattgttgggtggcgtcactatgatgttgctaagaagttaaaggaattaaaaaaggaggaactctttactatgaagttaatagaacctaagaaggcatttgaaatagagctgaggtcaaaggctggaaagtcatcaggagaaaaaattggttgtggaagggcaacacttcgcctgagatcaaaaggtcctgccaccgtggaagaaatgccttctgaaaccaaagcaaaggcaattgaaaagattgatgatgttcttgagttgtacatgggaattcgagatattgatttagccaccacaatgtttgaagctggaaaggacaaagtaaatccagatgaatttgctgtggcacttgacgaaactcttggagactttgcgttcccagacgaatttgtctttgatgtttggggagtcattggtgatgccaaacgaagaggattatgatgtgtacactccatctctgaagaaacaacccatcgttcttttttttctcttttttaaaaagtcctataagatctgtttttggacacctttactaactctggtttaatttcatgtgtatggaatatattctttgaaatataattttggtaattttgatttctgggcactttttaacattgctgatgtagtatgcttaagagaaatgacctaaataaggatcaattgtaatattcattcaaaaggtttttaaaagtaagttttaaggagtatttctcgacagatgattttcttctccattaatacccatgctttgtttttcacatataaatagatgatttcaatagctttgtagttttttttcaaaatcttaatgtaaactaggattggagtatgatttacctcatagtatcttcactgtgttatcctagtttgaacaggatatgttcaggaaatgaaaggtgaattatgccccttattggtctagaagcattggtttttttttgatcctctgagtaagggaaggaagattcctgtggcatgttgatttctatggccaactttctctctttgtaaaacaaagattgattgattttcttgtatgcttctttatacatgctcttaaaacttgtggttgaaatctgtgctgtgtgtgtgtgtctctgcatgtgtaaaagaagggtaaaaaagagatgggtagggtagaactacttgcatttgatttttttttcagtccttaaaacattatttctttttttcactcatagccacttttatgttaccttttataagcactccagggaagattttatatttttttgtagagtttttggaaagatatttactcaaaataactatgctttagaactttttttttttttttttttttggttaaacagcagcactttttagttccctaaactttagaagaaatgctatcagaaatagatttccttccagtggaaagaaaggaggaagggagaattgagaaaatatattagtctattattttagagttctaatacactctaaattatagagaaaattgagagtaaatgactgtcttatcactcttatttgacatttcgtaggtgtaagagaaatggaaatgaatggtttcaacaaagatcatttaatacagcagagcatggcatgaccaagcatctttgtaaagtgttagatggaaaatgctgtgtgctgccatggtaatcagaaataataacctgttagggatgtattctaggaaatcagaagtagttctcttttcttgctggattattgcttagataactcttgttttctggtaaaactttagttgtattgccatccactcctttttcaaatgagtttaatgccataaagctgatattctttgtccgattaatttgaaatctgcacagaagctgttttagtcattaatgtgtaacaaaagtagcttatagaatatggactgccttattgctgttgcttatcatttgaaaataaaatccagctcaggttgtaacttgttggtacttgtttagtgagttcgttttggaggttctgaaatacctattaattcttaatattacctctcttggaaagacacaaaaaaagagctgttatttttaggcataagatgacttaggaggcccatagaaacgttctcgtttaaacaacaacaacaaaataaatttatcagcactagaaaaaaaaaaaaaaaa

SEQ ID NO: 6 - protein, 315 aa, Homo sapiens
MPLKLRGKKKAKSKETAGLVEGEPTGAGGGSLSASRAPARRLVFHAQLAHGSATGRVEGFSSIQELYAQIAGAFEISPSEILYCTLNTPKIDMERLLGGQLGLEDFIFAHVKGIEKEVNVYKSEDSLGLTITDNGVGYAFIKRIKDGGVIDSVKTICVGDHIESINGENIVGWRHYDVAKKLKELKKEELFTMKLIEPKKAFEIELRSKAGKSSGEKIGCGRATLRLRSKGPATVEEMPSETKAKAIEKIDDVLELYMGIRDIDLATTMFEAGKDKVNPDEFAVALDETLGDFAFPDEFVFDVWGVIGDAKRRGL

SEQ ID NO: 7 - DNA, 4018 bp, Homo sapiens
  CDS (247)..(2496)
gttccgcccgccgctcttccagctgagactcggcagcggcggtcggagcggctgccacgccgcacctgcccgcgagggggcgcgccaggcccaggccgcagccgactgccgcgctctctgtccccgccctccgccatccccggactgcgctcacctgtctgggccgctggcctgggaggcgggggccggcgggagccaagccgaggaaagggcggagcggctctccgggcgcgtcatcggagcaccatggcggagctaggagctggcggcgacggccacaggggcggcgacggcgcagtgcgaagcgaaacagcacccgacagctacaaagtgcaagataagaaaaatgcctccagccgccctgcctctgcaatttcaggacaaaataacaaccactcaggaaataaaccagaccctccgcctgtgttacgtgttgatgaccggcagcggctggcccgggagcgacgtgaggaacgggagaaacagctagctgcaagagaaatagtgtggttagaaagagaagagcgagccaggcagcactacgagaagcacctggaagagcggaagaagaggttggaggagcagaggcagaaggaggagcggaggagggctgctgtggaggagaagcggaggcagagacttgaggaggacaaagaacgccacgaagctgttgtacggcgcacaatggaaaggagccagaagccaaaacagaagcataaccgttggtcgtggggaggctctctccatgggagccctagcatccacagtgcagatccagacaggcggtcagtttccaccatgaatctttcgaaatatgttgatcccgtcattagcaagcggctctcctcttcatctgcaactttactaaattctccagatagagctcgccgcctgcagctcagcccatgggagagcagcgttgttaacagactcctgacgcccacacattcgttcctggccagaagtaaaagcacagctgccttgtctggagaagcagcatcttgcagccccatcatcatgccctacaaagctgcacactctagaaattcgatggatcgaccaaaactctttgtaacaccacctgagggctcttctcgcaggaggatcattcatggcacagcgagctataaaaaagaaagagagagagaaaatgtactcttcctcacatctggcacccgaagggctgtatctccatctaatcccaaagcaagacaaccagctcgctcccgactttggcttccgtccaagtctcttcctcatttgcctggcacacccagaccgacatcctccttgccacccggctcagtcaaagctgctcctgctcaggtccggcccccatcccccggcaacatccgccctgtcaagagggaagtcaaagtggagcctgagaagaaagatcctgagaaggaacctcagaaagttgccaatgagccctcactaaagggcagagcacctttagtgaaggtagaagaagccacagttgaagagcggacacctgctgaaccagaagttggccctgctgctccagccatggccccagctccagcctcggccccagctccagcctcggccccagctccagccccggtccccaccccagccatggtctcagccccgtcatccactgtgaatgccagtgcttctgttaagacttctgcaggcaccaccgacccagaggaggccacaaggcttctagctgagaagaggcggctggcccgagagcagagagaaaaggaagaaagggagaggagggagcaggaagagcttgaaagacaaaagagagaggaattggctcaacgtgtggctgaagagaggacgactcgccgtgaggaggagtcgcgcaggctggaagccgagcaggcccgggagaaggaggagcagctgcagcggcaggcggaggagcgggcgctgcgcgagcgggaggaggcagagcgcgcccagaggcagaaagaagaagaagctcgcgttcgtgaagaagcagagagggtccggcaggaacgagagaagcatttccagagagaagagcaagagcgcctggagagaaagaagcgacttgaggagattatgaaaagaaccaggagaacagaagctacagataagaaaaccagtgatcagagaaacggtgatatagccaagggagctctcactggaggaacagaggtgtctgcacttccatgtacaacaaacgctccgggaaatggaaagccagttggcagcccacatgtggttacctcacaccagtcaaaagtgacagtggagagcactcccgatttggaaaaacaaccaaatgaaaatggtgtatctgttcagaatgaaaattttgaagaaattataaacttacccattggatctaaaccatccagattagatgtcaccaacagtgagagcccagaaattcctttgaatccaattttggcctttgatgatgaagggacacttgggcccctgcctcaggtagatggtgttcagacacagcagactgcagaagttatatgagtgtttcttctgaagaaccaaagctgaaatttaatgagaatttctacaattaatggaattcctttcctgctataaaggagcatcccctccacccgttttctagagttcttgaccatcattttgaaaagatttattaaaactagctaaagacaacagactggatagcttttctaataattttcatcaataggaaaaaagaaatacgtctcattcttcaatactttaaaatggctttttccagtgtgctccttcttagcaatcaatatttttctgcattctttaaaagacaagagaatttggttataaaagaaatgggctgactaggcatgatttttttggtcttaaaagcttaacatgtaaaattggcaaaaaaaattttttaccttttataatacttgaaaaataagtacctctttgttctacaagtagaatgaataggagaagagtttaagcctgtttttttaaaatattattgcaaagagctctatttgtagaagcaaattataggcagattaccaggttcttataaatacagcttgtacatggacattctgcaaacccagctgtcacatttttcttgcaactccttttgcaaaagcagactaaaatgttttaaaatgtgaaaaaacattattttttcaaagcaagaaaataatttactgccctcttacataatgtatttataaagtttttccagataaactaatcaaataaattagaataatgtgacaacattacaaatttaatttgttagctgcattccttctgatgttaccacgatagaatgttactgatgattcagggctatttctgaagtctgtatgttgctgctgtccccagtgatggtggacttatctttgccttacctgatcacaaattatgttggggaaaataaagatttaatatttctttaaatagaaaaagaatttggttttgctcgtttaagagcaatgagaaaatgatggaatgttgactgtgtttggcacacaggacacggaccttcatggaagtccttgctctgcgtggcatctgtcagcttttcacctttcattcttattcttcacttttgctgctgagcctagctgtacaaacttgcactttcatttgctaatataaattcagttttattttaccattttagagactactaatgattaaatgtagaaggagagggtgcacatgtttttatgtggagtgtttaaaagataaatttataccactgtaatgtgcagcttttattaaaagagaaattggttgaactgctaggttgaatgagagacttcatctattggactattttttttaatccaggcatatggtctttagtaatggcttgtaatttgtgaaaacattaatttgggggttttccctgttttcagttgtccatgtacacatagtcattatattagaaaagaaagctgttcaacaaacttgtttaatttgtttaaatcaacatagcatgaaacaccaaataaaatgtttgacatagttttaaaaaaaaaaaaaaaaaaaaaaaaaaaaa

SEQ ID NO: 8 - protein, 749 aa, Homo sapiens
MAELGAGGDGHRGGDGAVRSETAPDSYKVQDKKNASSRPASAISGQNNNHSGNKPDPPPVLRVDDRQRLARERREEREKQLAAREIVWLEREERARQHYEKHLEERKKRLEEQRQKEERRRAAVEEKRRQRLEEDKERHEAVVRRTMERSQKPKQKHNRWSWGGSLHGSPSIHSADPDRRSVSTMNLSKYVDPVISKRLSSSSATLLNSPDRARRLQLSPWESSVVNRLLTPTHSFLARSKSTAALSGEAASCSPIIMPYKAAHSRNSMDRPKLFVTPPEGSSRRRIIHGTASYKKERERENVLFLTSGTRRAVSPSNPKARQPARSRLWLPSKSLPHLPGTPRPTSSLPPGSVKAAPAQVRPPSPGNIRPVKREVKVEPEKKDPEKEPQKVANEPSLKGRAPLVKVEEATVEERTPAEPEVGPAAPAMAPAPASAPAPASAPAPAPVPTPAMVSAPSSTVNASASVKTSAGTTDPEEATRLLAEKRRLAREQREKEERERREQEELERQKREELAQRVAEERTTRREEESRRLEAEQAREKEEQLQRQAEERALREREEAERAQRQKEEEARVREEAERVRQEREKHFQREEQERLERKKRLEEIMKRTRRTEATDKKTSDQRNGDIAKGALTGGTEVSALPCTTNAPGNGKPVGSPHVVTSHQSKVTVESTPDLEKQPNENGVSVQNENFEEIINLPIGSKPSRLDVTNSESPEIPLNPILAFDDEGTLGPLPQVDGVQTQQTAEVI

SEQ ID NO: 9 - DNA, 2651 bp, Homo sapiens
  CDS (219)..(1841)
tctttttcttttttttttaagaaaaactagtgacattgcagagaaggacgcttcctctctatcttttggcgcattagtgaagggggtattctattttgttaaagcgcccaagggggcgcagggaccttggagagaagagtggggaggaaagaggaagggtgggtggggggcagagggcgagtcggcggcggcgagggcaagctctttcttgcggcacgatgccgtctctgctggtgctcactttctccccgtgcgtactactcggctgggcgttgctggccggcggcaccggtggcggtggcgttggcggcggcggcggtggcgcgggcataggcggcggacgccaggagagagaggcgctgccgccacagaagatcgaggtgctggtgttactgccccaggatgactcgtacttgttttcactcacccgggtgcggccggccatcgagtatgctctgcgcagcgtggagggcaacgggactgggaggcggcttctgccgccgggcactcgcttccaggtggcttacgaggattcagactgtgggaaccgtgcgctcttcagcttggtggaccgcgtggcggcggcgcggggcgccaagccagaccttatcctggggccagtgtgcgagtatgcagcagcgccagtggcccggcttgcatcgcactgggacctgcccatgctgtcggctggggcgctggccgctggcttccagcacaaggactctgagtactcgcacctcacgcgcgtggcgcccgcctacgccaagatgggcgagatgatgctcgccctgttccgccaccaccactggagccgcgctgcactggtctacagcgacgacaagctggagcggaactgctacttcaccctcgagggggtccacgaggtcttccaggaggagggtttgcacacgtccatctacagtttcgacgagaccaaagacttggatctggaagacatcgtgcgcaatatccaggccagtgagagagtggtgatcatgtgtgcgagcagtgacaccatccggagcatcatgctggtggcgcacaggcatggcatgaccagtggagactacgccttcttcaacattgagctcttcaacagctcttcctatggagatggctcatggaagagaggagacaaacacgactttgaagctaagcaagcatactcgtccctccagacagtcactctactgaggacagtgaaacctgagtttgagaagttttccatggaggtgaaaagttcagttgagaaacaagggctcaatatggaggattacgttaacatgtttgttgaaggattccacgatgccatcctcctctacgtcttggctctacatgaagtactcagagctggttacagcaaaaaggatggagggaaaattatacagcagacttggaacagaacatttgaaggtatcgccgggcaggtgtccatagatgccaacggagaccgatatggggatttctctgtgattgccatgactgatgtggaggcgggcacccaggaggttattggtgattattttggaaaagaaggtcgttttgaaatgcggccgaatgtcaaatatccttggggccctttaaaactgagaatagatgaaaaccgaattgtagagcatacaaacagctctccctgcaaatcatgtggcctagaagaatcggcagtgacaggaattgtcgtgggggctttactaggagctggcttgctaatggccttctactttttcaggaagaaatacagaataaccattgagaggcgaacccagcaagaagaaagtaaccttggaaaacatcgggaattacgggaagattccatcagatcccatttttcagtagcttaaaggaagccccccactttttttttttctgcctgagattctttaaggagatagacgggttgaaagacatcaatgaaacagaaggggcgttcttgaagaattcataattttaagcagttagtaatttcattttaaaatttctgtagaagctcaggaattatgattaatcaccatctgcctccaggcctttcatctcatgacaaacaaatataataatgatatcgtgtcactctgttaaatgttcatactgtttcaagcccatatgattagatttatgtttttaaaatctgttgtctccatatcttgatggcttttgggagcatttcacacaaggatataaaatgcggttttcttaaatgaaatgttttgtagctagaataaaatcatttttacaagtacagcattcttggaaagaatttaacacccaaaaaggggaaaatgtaatgaaaaatctcaaggttggaaatacagccttactctctctagagctggaggacaggtttgtggttgaggacttctctgtccgatgtctacattcaggttctgacttcatatcttgaaaaaggatttcctccctgtctttttcagtgtctcataaacgctactctggattgttgtaaatattagtgagatgggaggatttacagaagaaaagcaagtcaaaaatatttcctttttgatgtaaaaaaaaaaagccctatttcgcactaacattttattttacaagtattttaatcttatattttggtattagaaaaatttgtctattttttcattttgaagattaaatgttgcttacattttaaaaaaaaa

SEQ ID NO: 10 - protein, 540 aa, Homo sapiens
MPSLLVLTFSPCVLLGWALLAGGTGGGGVGGGGGGAGIGGGRQEREALPPQKIEVLVLLPQDDSYLFSLTRVRPAIEYALRSVEGNGTGRRLLPPGTRFQVAYEDSDCGNRALFSLVDRVAAARGAKPDLILGPVCEYAAAPVARLASHWDLPMLSAGALAAGFQHKDSEYSHLTRVAPAYAKMGEMMLALFRHHHWSRAALVYSDDKLERNCYFTLEGVHEVFQEEGLHTSIYSFDETKDLDLEDIVRNIQASERVVIMCASSDTIRSIMLVAHRHGMTSGDYAFFNIELFNSSSYGDGSWKRGDKHDFEAKQAYSSLQTVTLLRTVKPEFEKFSMEVKSSVEKQGLNMEDYVNMFVEGFHDAILLYVLALHEVLRAGYSKKDGGKIIQQTWNRTFEGIAGQVSIDANGDRYGDFSVIAMTDVEAGTQEVIGDYFGKEGRFEMRPNVKYPWGPLKLRIDENRIVEHTNSSPCKSCGLEESAVTGIVVGALLGAGLLMAFYFFRKKYRITIERRTQQEESNLGKHRELREDSIRSHFSVA

SEQ ID NO: 11 - DNA, 19 bp, Artificial sequence
  Synthesized oligonucleotide primer
acttttaccgaatgccacc

SEQ ID NO: 12 - DNA, 23 bp, Artificial sequence
  Synthesized oligonucleotide primer
tggttactctcaagtcaatcttg

SEQ ID NO: 13 - DNA, 20 bp, Artificial sequence
  Synthesized oligonucleotide primer
ccatcaagaccatcaccatc

SEQ ID NO: 14 - DNA, 20 bp, Artificial sequence
  Synthesized oligonucleotide primer
gccgataaacaggtacacga

SEQ ID NO: 15 - DNA, 20 bp, Artificial sequence
  Synthesized oligonucleotide primer
cctgagatcaaaaggtcctg

SEQ ID NO: 16 - DNA, 19 bp, Artificial sequence
  Synthesized oligonucleotide primer
cttcaaacattgtggtggc

SEQ ID NO: 17 - DNA, 23 bp, Artificial sequence
  Synthesized oligonucleotide primer
gctacagataagaaaaccagtga

SEQ ID NO: 18 - DNA, 17 bp, Artificial sequence
  Synthesized oligonucleotide primer
gctttccatttcccgga

SEQ ID NO: 19 - DNA, 18 bp, Artificial sequence
  Synthesized oligonucleotide primer
tcggcagtgacaggaatt

SEQ ID NO: 20 - DNA, 18 bp, Artificial sequence
  Synthesized oligonucleotide primer
cccgatgttttccaaggt